SEQUENCE LISTING (54 sequences)

SEQ ID NO: 1 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGSVQLADHYQQNTPIGDGPVLLPDNHYLDTESALSKDPNEKRDHMVLLEEVEAAGITLGMDELYK

SEQ ID NO: 3 - protein, 239 aa, synthetic construct
MVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGGVQLADHYQQNTPIGDGPVLLPDNHYLDTESALSKDPNEKRDHMVLLEEVEAAGITLGMDELYK

SEQ ID NO: 4 - protein, 239 aa, synthetic construct
MVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNDHNVYITADKQKNGIKANFKIRHNIEDGGVQLADHYQQNTPIGDGPVLLPDNHYLDTESALSKDPNEKRDHMVLLEEVEAAGITLGMDELYK

SEQ ID NO: 5 - protein, 236 aa, synthetic construct
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIKLDIVSHNEDYTIVEQYERAEGRHSTGGMDELYK

SEQ ID NO: 6 - protein, 236 aa, synthetic construct
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 7 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 8 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGSVQLADHYQQNTPIGDGPVLLPDNHYLDTESALSKDPNEKRDHMVLLEEVEAAGITLGMDELYK

SEQ ID NO: 9 - protein, 238 aa, synthetic construct
VSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGGVQLADHYQQNTPIGDGPVLLPDNHYLDTESALSKDPNEKRDHMVLLEEVEAAGITLGMDELYK

SEQ ID NO: 10 - protein, 238 aa, synthetic construct
VSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNDHNVYITADKQKNGIKANFKIRHNIEDGGVQLADHYQQNTPIGDGPVLLPDNHYLDTESALSKDPNEKRDHMVLLEEVEAAGITLGMDELYK

SEQ ID NO: 11 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIKLDIVSHNEDYTIVEQYERAEGRHSTGGMDELYK

SEQ ID NO: 12 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 13 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacgaccacaacgtctatatcacggccgacaagcagaagaacggcatcaaggcgaacttcaagatccgccacaacatcgaggacggcggcgtgcagctcgccgaccactaccagcagaacacccccatcggcgacggccccgtgctgctgcccgacaaccactacctggacaccgaatccgccctgagcaaagaccccaacgagaagcgcgatcacatggtcctgctggaggaggtggaggccgccgggatcactctcggcatggacgagctgtacaag

SEQ ID NO: 14 - DNA, 708 bp, synthetic construct
  source 1..708: mol_type = other DNA
atggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaagaagaccatgggctgggaggagtccgaggagcggatgtaccccgaggacggcgccctgaagagcgagatcaagaagaggctgaagctgaaggacggcggccactacgccgccgaggtcaagaccacctacaaggccaagaagcccgtgcagctgcccggcgcctacatcgtcgacatcgacttggacatcgtgtcccacaacgaggactacaccatcgtggaacagtacgaggacgccgagggccgccactccaccggcggcatggacgagctgtacaag

SEQ ID NO: 15 - protein, 4 aa, synthetic construct
KDEL

SEQ ID NO: 16 - protein, 18 aa, synthetic construct
MLLSVPLLLGLLGLAAAD

SEQ ID NO: 17 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGSVQLADHYQQNTPIGDGPVLLPDNHYLDTESALSKDPNEKRDHMVLLEDVEAAGITLGMDELYK

SEQ ID NO: 18 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGSVQLADHYQQNTPIGDGPVLLPDNHYLDTDSALSKDPNEKRDHMVLLEEVEAAGITLGMDELYK

SEQ ID NO: 19 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNDHNVYITADKQKNGIKANFKIRHNIEDGSVQLADHYQQNTPIGDGPVLLPDNHYLDTESALSKDPNEKRDHMVLLEEVEAAGITLGMDELYK

SEQ ID NO: 20 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHDVYITADKQKNGIKANFKIRHNIEDGSVQLADHYQQNTPIGDGPVLLPDNHYLDTESALSKDPNEKRDHMVLLEEVEAAGITLGMDELYK

SEQ ID NO: 21 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHEVYITADKQKNGIKANFKIRHNIEDGSVQLADHYQQNTPIGDGPVLLPDNHYLDTDSALSKDPNEKRDHMVLLEEVEAAGITLGMDELYK

SEQ ID NO: 22 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHEVYITADKQKNGIKANFKIRHNIEDGSVQLADHYQQNTPIGDGPVLLPDNHYLDTDSALSKDPNEKRDHMVLLEDVEAAGITLGMDELYK

SEQ ID NO: 23 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIDLDIVSHNEDYTIVEQYERAEGRHSTGGMDELYK

SEQ ID NO: 24 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 25 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEEAEGRHSTGGMDELYK

SEQ ID NO: 26 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEDSEERMYPEDGALKSEIKKRLKLKDGGHYAAEVKTTYKAKKPVQLPGAYIVDIDLDIVSHNEDYTIVEQYEEAEGRHSTGGMDELYK

SEQ ID NO: 27 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESDERMYPEDGALKSEIKKRLKLKDGGHYAAEVKTTYKAKKPVQLPGAYIVDIDLDIVSHNEDYTIVEQYEEAEGRHSTGGMDELYK

SEQ ID NO: 28 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEDSDERMYPEDGALKSEIKKRLKLKDGGHYAAEVKTTYKAKKPVQLPGAYIVDIDLDIVSHNEDYTIVEQYEEAEGRHSTGGMDELYK

SEQ ID NO: 29 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEEADGRHSTGGMDELYK

SEQ ID NO: 30 - protein, 231 aa, synthetic construct
EENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIELDIVSHNEDYTIVEQYEEAEGRHSTGGMDELYK

SEQ ID NO: 31 - protein, 231 aa, synthetic construct
EEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 32 - protein, 231 aa, synthetic construct
EEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEESEERMYPEDGALKGEIKQRLKLKDGGHYDAEVKTTYKAKKPVQLPGAYNVDIDLDITSHNEDYTIVEQYEEAEGRHSTGGMDELYK

SEQ ID NO: 33 - protein, 236 aa, synthetic construct
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKLRLKLKDGGHYAAEVKTTYKAKKPVQLPGAYIVDIDLDIVSHNEDYTIVEQYEDAEGRHSTGGMDELYK

SEQ ID NO: 34 - protein, 78 aa, synthetic construct
MLCQQMIRTTAKRSSNIMTRPIIMKRSVHFKDGVYENIPFKVKGRKTPYALSHFGFFAIGFAVPFVACYVQLKKSGAF

SEQ ID NO: 35 - protein, 263 aa, synthetic construct
MLLSVPLLLGLLGLAAADGDPATMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYKKDEL

SEQ ID NO: 36 - protein, 392 aa, synthetic construct
MLCQQMIRTTAKRSSNIMTRPIIMKRSVHFKDGVYENIPFKVKGRKTPYALSHFGFFAIGFAVPFVACYVQLKKSGAFMLCQQMIRTTAKRSSNIMTRPIIMKRSVHFKDGVYENIPFKVKGRKTPYALSHFGFFAIGFAVPFVACYVQLKKSGAFMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKLRLKLKDGGHYAAEVKTTYKAKKPVQLPGAYIVDIDLDIVSHNEDYTIVEQYEEAEGRHSTGGMDELYK

SEQ ID NO: 37 - protein, 1324 aa, synthetic construct
MAFYSCCWVLLALTWHTSAYGPDQRAQKKGDIILGGLFPIHFGVAAKDQDLKSRPESVECIRYNFRGFRWLQAMIFAIEEINSSPALLPNLTLGYRIFDTCNTVSKALEATLSFVAQNKIDSLNLDEFCNCSEHIPSTIAVVGATGSGVSTAVANLLGLFYIPQVSYASSSRLLSNKNQFKSFLRTIPNDEHQATAMADIIEYFRWNWVGTIAADDDYGRPGIEKFREEAEERDICIDFSELISQYSDEEEIQHVVEVIQNSTAKVIVVFSSGPDLEPLIKEIVRRNITGKIWLASEAWASSSLIAMPQYFHVVGGTIGFALKAGQIPGFREFLKKVHPRKSVHNGFAKEFWEETFNCHLQEGAKGPLPVDTFLRGHEESGDRFSNSSTAFRPLCTGDENISSVETPYIDYTHLRISYNVYLAVYSIAHALQDIYTCLPGRGLFTNGSCADIKKVEAWQVLKHLRHLNFTNNMGEQVTFDECGDLVGNYSIINWHLSPEDGSIVFKEVGYYNVYAKKGERLFINEEKILWSGFSREPLTFVLSVLQVPFSNCSRDCLAGTRKGIIEGEPTCCFECVECPDGEYSDETDASACNKCPDDFWSNENHTSCIAKEIEFLSWTEPFGIALTLFAVLGIFLTAFVLGVFIKFRNTPIVKATNRELSYLLLFSLLCCFSSSLFFIGEPQDWTCRLRQPAFGISFVLCISCILVKTNRVLLVFEAKIPTSFHRKWWGLNLQFLLVFLCTFMQIVICVIWLYTAPPSSYRNQELEDEIIFITCHEGSLMALGFLIGYTCLLAAICFFFAFKSRKLPENFNEAKFITFSMLIFFIVWISFIPAYASTYGKFVSAVEVIAILAASFGLLACIFFNKIYIILFKPSRNTIEEVRCSTAAHAFKVAARATLRRSNVSRKRSSSLGGSTGSTPSSSISSKSNSEDPFPQPERQKQQQPLALTQQEQQQQPLTLPQQQRSQQQPRCKQKVIFGSGTVTFSLSFDEPQKNAMAHRNSTHQNSLEAQKSSDTLTRHQPLLPLQCGETDLDLTVQETGLQGPVGGDQRPEVEDPEELSPALVVSSSQSFVISGGGSTVTENVVNSMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 38 - protein, 1088 aa, synthetic construct
MAFYSCCWVLLALTWHTSAYGPDQRAQKKGDIILGGLFPIHFGVAAKDQDLKSRPESVECIRYNFRGFRWLQAMIFAIEEINSSPALLPNLTLGYRIFDTCNTVSKALEATLSFVAQNKIDSLNLDEFCNCSEHIPSTIAVVGATGSGVSTAVANLLGLFYIPQVSYASSSRLLSNKNQFKSFLRTIPNDEHQATAMADIIEYFRWNWVGTIAADDDYGRPGIEKFREEAEERDICIDFSELISQYSDEEEIQHVVEVIQNSTAKVIVVFSSGPDLEPLIKEIVRRNITGKIWLASEAWASSSLIAMPQYFHVVGGTIGFALKAGQIPGFREFLKKVHPRKSVHNGFAKEFWEETFNCHLQEGAKGPLPVDTFLRGHEESGDRFSNSSTAFRPLCTGDENISSVETPYIDYTHLRISYNVYLAVYSIAHALQDIYTCLPGRGLFTNGSCADIKKVEAWQVLKHLRHLNFTNNMGEQVTFDECGDLVGNYSIINWHLSPEDGSIVFKEVGYYNVYAKKGERLFINEEKILWSGFSREPLTFVLSVLQVPFSNCSRDCLAGTRKGIIEGEPTCCFECVECPDGEYSDETDASACNKCPDDFWSNENHTSCIAKEIEFLSWTEPFGIALTLFAVLGIFLTAFVLGVFIKFRNTPIVKATNRELSYLLLFSLLCCFSSSLFFIGEPQDWTCRLRQPAFGISFVLCISCILVKTNRVLLVFEAKIPTSFHRKWWGLNLQFLLVFLCTFMQIVICVIWLYTAPPSSYRNQELEDEIIFITCHEGSLMALGFLIGYTCLLAAICFFFAFKSRKLPENFNEAKFITFSMLIFFIVWISFIPAYASTYGKFVSAVEVIAILAASFGLLACIFFNKIYIILFKPSRNTIEEVRCSTAAHAFKVAARATLRRSNVSRKRSSSLGGSTGSTPSSSISSKSNSEDPFPQPERQKQQQPLALTQQEQQQQPLTLPQQQRSQQQPRCKQKVIFGSGTVTFSLSFDEPQKNAMAHRNSTHQNSLEAQKSSDTLTRHQPLLPLQCGETDLDLTVQETGLQGPVGGDQRPEVEDPEELSPALVVSSSQSFVISGGGSTVTENVVNS

SEQ ID NO: 39 - protein, 1430 aa, synthetic construct
MVGLLLFFFPAIFLEVSLLPRSPGRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGINRCLPDGQSLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKAGAGNANSNGKSVSWSEPGGGQVPKGQHMWHRLSVHVKTNETACNQTAVIKPLTKSYQGSGKSLTFSDTSTKTLYNVEEEEDAQPIRFSPPGSPSMVVHRRVPSAATTPPLPSHLTAEETPLFLAEPALPKGLPPPLQQQQQPPPQQKSLMDQLQGVVSNFSTAIPDFHAVLAGPGGPGNGLRSLYPPPPPPQHLQMLPLQLSTFGEELVSPPADDDDDSERFKLLQEYVYEHEREGNTEEDELEEEEEDLQAASKLTPDDSPALTPPSPFRDSVASGSSVPSSPVSESVLCTPPNVSYASVILRDYKQSSSTLMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 40 - protein, 1194 aa, synthetic construct
MVGLLLFFFPAIFLEVSLLPRSPGRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGINRCLPDGQSLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKAGAGNANSNGKSVSWSEPGGGQVPKGQHMWHRLSVHVKTNETACNQTAVIKPLTKSYQGSGKSLTFSDTSTKTLYNVEEEEDAQPIRFSPPGSPSMVVHRRVPSAATTPPLPSHLTAEETPLFLAEPALPKGLPPPLQQQQQPPPQQKSLMDQLQGVVSNFSTAIPDFHAVLAGPGGPGNGLRSLYPPPPPPQHLQMLPLQLSTFGEELVSPPADDDDDSERFKLLQEYVYEHEREGNTEEDELEEEEEDLQAASKLTPDDSPALTPPSPFRDSVASGSSVPSSPVSESVLCTPPNVSYASVILRDYKQSSSTL

SEQ ID NO: 41 - protein, 1204 aa, synthetic construct
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHVMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 42 - protein, 968 aa, synthetic construct
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 43 - protein, 1700 aa, synthetic construct
MGRLGYWTLLVLPALLVWRDPAQNAAAEKGPPALNIAVLLGHSHDVTERELRNLWGPEQATGLPLDVNVVALLMNRTDPKSLITHVCDLMSGARIHGLVFGDDTDQEAVAQMLDFISSQTFIPILGIHGGASMIMADKDPTSTFFQFGASIQQQATVMLKIMQDYDWHVFSLVTTIFPGYRDFISFIKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGLGILTTAASSMLEKFSYIPEAKASCYGQAEKPETPLHTLHQFMVNVTWDGKDLSFTEEGYQVHPRLVVIVLNKDREWEKVGKWENQTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTRFNQRGVEDALVSLKTGKLDAFIYDAAVLNYKAGRDEGCKLVTIGSGYIFASTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLFSISRGIYSCIHGVHIEEKKKSPDFNLTGSQSNMLKLLRSAKNISNMSNMNSSRMDSPKRATDFIQRGSLIVDMVSDKGNLIYSDNRSFQGKDSIFGDNMNELQTFVANRHKDNLSNYVFQGQHPLTLNESNPNTVEVAVSTESKGNSRPRQLWKKSMESLRQDSLNQNPVSQRDEKTAENRTHSLKSPRYLPEEVAHSDISETSSRATCHREPDNNKNHKTKDNFKRSMASKYPKDCSDVDRTYMKTKASSPRDKIYTIDGEKEPSFHLDPPQFVENITLPENVGFPDTYQDHNENFRKGDSTLPMNRNPLHNEDGLPNNDQYKLYAKHFTLKDKGSPHSEGSDRYRQNSTHCRSCLSNLPTYSGHFTMRSPFKCDACLRMGNLYDIDEDQMLQETGNPATREEVYQQDWSQNNALQFQKNKLRINRQHSYDNILDKPREIDLSRPSRSISLKDRERLLEGNLYGSLFSVPSSKLLGNKSSLFPQGLEDSKRSKSLLPDHASDNPFLHTYGDDQRLVIGRCPSDPYKHSLPSQAVNDSYLRSSLRSTASYCSRDSRGHSDVYISEHVMPYAANKNTMYSTPRVLNSCSNRRVYKKMPSIESDVMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 44 - protein, 1464 aa, synthetic construct
MGRLGYWTLLVLPALLVWRDPAQNAAAEKGPPALNIAVLLGHSHDVTERELRNLWGPEQATGLPLDVNVVALLMNRTDPKSLITHVCDLMSGARIHGLVFGDDTDQEAVAQMLDFISSQTFIPILGIHGGASMIMADKDPTSTFFQFGASIQQQATVMLKIMQDYDWHVFSLVTTIFPGYRDFISFIKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGLGILTTAASSMLEKFSYIPEAKASCYGQAEKPETPLHTLHQFMVNVTWDGKDLSFTEEGYQVHPRLVVIVLNKDREWEKVGKWENQTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTRFNQRGVEDALVSLKTGKLDAFIYDAAVLNYKAGRDEGCKLVTIGSGYIFASTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLFSISRGIYSCIHGVHIEEKKKSPDFNLTGSQSNMLKLLRSAKNISNMSNMNSSRMDSPKRATDFIQRGSLIVDMVSDKGNLIYSDNRSFQGKDSIFGDNMNELQTFVANRHKDNLSNYVFQGQHPLTLNESNPNTVEVAVSTESKGNSRPRQLWKKSMESLRQDSLNQNPVSQRDEKTAENRTHSLKSPRYLPEEVAHSDISETSSRATCHREPDNNKNHKTKDNFKRSMASKYPKDCSDVDRTYMKTKASSPRDKIYTIDGEKEPSFHLDPPQFVENITLPENVGFPDTYQDHNENFRKGDSTLPMNRNPLHNEDGLPNNDQYKLYAKHFTLKDKGSPHSEGSDRYRQNSTHCRSCLSNLPTYSGHFTMRSPFKCDACLRMGNLYDIDEDQMLQETGNPATREEVYQQDWSQNNALQFQKNKLRINRQHSYDNILDKPREIDLSRPSRSISLKDRERLLEGNLYGSLFSVPSSKLLGNKSSLFPQGLEDSKRSKSLLPDHASDNPFLHTYGDDQRLVIGRCPSDPYKHSLPSQAVNDSYLRSSLRSTASYCSRDSRGHSDVYISEHVMPYAANKNTMYSTPRVLNSCSNRRVYKKMPSIESDV

SEQ ID NO: 45 - protein, 1141 aa, synthetic construct
QHIFAFFCTGFLGAVVGANFPNNIQIGGLFPNQQSQEHAAFRFALSQLTEPPKLLPQIDIVNISDSFEMTYRFCSQFSKGVYAIFGFYERRTVNMLTSFCGALHVCFITPSFPVDTSNQFVLQLRPELQDALISIIDHYKWQKFVYIYDADRGLSVLQKVLDTAAEKNWQVTAVNILTTTEEGYRMLFQDLEKKKERLVVVDCESERLNAILGQIIKLEKNGIGYHYILANLGFMDIDLNKFKESGANVTGFQLVNYTDTIPAKIMQQWKNSDARDHTRVDWKRPKYTSALTYDGVKVMAEAFQSLRRQRIDISRRGNAGDCLANPAVPWGQGIDIQRALQQVRFEGLTGNVQFNEKGRRTNYTLHVIEMKHDGIRKIGYWNEDDKFVPAATDAQAGGDNSSVQNRTYIVTTILEDPYVMLKKNANQFEGNDRYEGYCVELAAEIAKHVGYSYRLEIVSDGKYGARDPDTKAWNGMVGELVYGRADVAVAPLTITLVREEVIDFSKPFMSLGISIMIKKPQKSKPGVFSFLDPLAYEIWMCIVFAYIGVSVVLFLVSRFSPYEWHSEEFEEGRDQTTSDQSNEFGIFNSLWFSLGAFMQQGCDISPRSLSGRIVGGVWWFFTLIIISSYTANLAAFLTVERMVSPIESAEDLAKQTEIAYGTLEAGSTKEFFRRSKIAVFEKMWTYMKSAEPSVFVRTTEEGMIRVRKSKGKYAYLLESTMNEYIEQRKPCDTMKVGGNLDSKGYGIATPKGSALRNPVNLAVLKLNEQGLLDKLKNKWWYDKGECGSGGGDSKDKTSALSLSNVAGVFYILIGGLGLAMLVALIEFCYKSRSESKRMKGFCLIPQQSINEAIRTSTLPRNSGAGASSGGSGENGRVVSHDFPKSMQSIPCMSHSSGMPLGATGLMVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEESEERMYPEDGALKSEIKKRLKLKDGGHYAAEVKTTYKAKKPVQLPGAYIVDIDLDIVSHNEDYTIVEQYEDAEGRHSTGGMDELYK

SEQ ID NO: 46 - protein, 905 aa, synthetic construct
QHIFAFFCTGFLGAVVGANFPNNIQIGGLFPNQQSQEHAAFRFALSQLTEPPKLLPQIDIVNISDSFEMTYRFCSQFSKGVYAIFGFYERRTVNMLTSFCGALHVCFITPSFPVDTSNQFVLQLRPELQDALISIIDHYKWQKFVYIYDADRGLSVLQKVLDTAAEKNWQVTAVNILTTTEEGYRMLFQDLEKKKERLVVVDCESERLNAILGQIIKLEKNGIGYHYILANLGFMDIDLNKFKESGANVTGFQLVNYTDTIPAKIMQQWKNSDARDHTRVDWKRPKYTSALTYDGVKVMAEAFQSLRRQRIDISRRGNAGDCLANPAVPWGQGIDIQRALQQVRFEGLTGNVQFNEKGRRTNYTLHVIEMKHDGIRKIGYWNEDDKFVPAATDAQAGGDNSSVQNRTYIVTTILEDPYVMLKKNANQFEGNDRYEGYCVELAAEIAKHVGYSYRLEIVSDGKYGARDPDTKAWNGMVGELVYGRADVAVAPLTITLVREEVIDFSKPFMSLGISIMIKKPQKSKPGVFSFLDPLAYEIWMCIVFAYIGVSVVLFLVSRFSPYEWHSEEFEEGRDQTTSDQSNEFGIFNSLWFSLGAFMQQGCDISPRSLSGRIVGGVWWFFTLIIISSYTANLAAFLTVERMVSPIESAEDLAKQTEIAYGTLEAGSTKEFFRRSKIAVFEKMWTYMKSAEPSVFVRTTEEGMIRVRKSKGKYAYLLESTMNEYIEQRKPCDTMKVGGNLDSKGYGIATPKGSALRNPVNLAVLKLNEQGLLDKLKNKWWYDKGECGSGGGDSKDKTSALSLSNVAGVFYILIGGLGLAMLVALIEFCYKSRSESKRMKGFCLIPQQSINEAIRTSTLPRNSGAGASSGGSGENGRVVSHDFPKSMQSIPCMSHSSGMPLGATGL

SEQ ID NO: 47 - protein, 449 aa, synthetic construct
MGSHHHHHHGMASMTGGQQMGRDLYDDDDKDLATMVDSSRRKWNKTGHAVRVIGRLSSLENVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKELFSLFDKDGDGTITTKELGTVMRSLGQNPTEAELQDMINEVDADGDGTIDFPEFLTMMARKMKYRDTEEEIREAFGVFDKDGNGYISAAELRHVMTNLGEKLTDEEVDEMIREADIDGDGQVNYEEFVQMMTA

SEQ ID NO: 48 - protein, 580 aa, synthetic construct
MATPAGRRASETERLLTPNPGYGTQVGTSPAPTTPTEEEDLRRRLKYFFMSPCDKFRAKGRKPCKLMLQVVKILVVTVQLILFGLSNQLVVTFREENTIAFRHLFLLGYSDGSDDTFAAYTQEQLYQAIFYAVDQYLILPEISLGRYAYVRGGGGPWANGSALALCQRYYHRGHVDPANDTFDIDPRVVTDCIQVDPPDRPPDIPSEDLDFLDGSASYKNLTLKFHKLINVTIHFQLKTINLQSLINNEIPDCYTFSILITFDNKAHSGRIPIRLETKTHIQECKHPSVSRHGDNSFRLLFDVVVILTCSLSFLLCARSLLRGFLLQNEFVVFMWRRRGREISLWERLEFVNGWYILLVTSDVLTISGTVMKIGIEAKNLASYDVCSILLGTSTLLVWVGVIRYLTFFHKYNILIATLRVALPSVMRFCCCVAVIYLGYCFCGWIVLGPYHVKFRSLSMVSECLFSLINGDDMFVTFAAMQAQQGHSSLVWLFSQLYLYSFISLFIYMVLSLFIALITGAYDTIKHPGGTGTEKSELQAYIEQCQDSPTSGKFRRGSGSACSLFCCCGRDSPEDHSLLVN

SEQ ID NO: 49 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHEVYITADKQKNGIKANFKIRHNIEDGSVQLADHYQQNTPIGDGPVLLPDNHYLDTDSALSKDPNEKRDHMVLLEDVEAAGITLGMDELYK

SEQ ID NO: 50 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHEVYITADKQKNGIKANFKIRHNIEDGSVQLADHYQQNTPIGDGPVLLPDNHYLDTDSALSKDPNEKRDHMVLLEEVEAAGITLGMDELYK

SEQ ID NO: 51 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGSVQLADHYQQNTPIGDGPVLLPDNHYLDTESALSKDPNEKRDHMVLLEDVEAAGITLGMDELYK

SEQ ID NO: 52 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHNVYITADKQKNGIKANFKIRHNIEDGSVQLADHYQQNTPIGDGPVLLPDNHYLDTDSALSKDPNEKRDHMVLLEEVEAAGITLGMDELYK

SEQ ID NO: 53 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNDHNVYITADKQKNGIKANFKIRHNIEDGSVQLADHYQQNTPIGDGPVLLPDNHYLDTESALSKDPNEKRDHMVLLEEVEAAGITLGMDELYK

SEQ ID NO: 54 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNEHDVYITADKQKNGIKANFKIRHNIEDGSVQLADHYQQNTPIGDGPVLLPDNHYLDTESALSKDPNEKRDHMVLLEEVEAAGITLGMDELYK

(19) United States
(12) Patent Application Publication
(10) Pub. No.: US 2024/0369485 A1
(43) Pub. Date: Nov. 7, 2024

(54) FLUORESCENT SENSOR FOR MONITORING CALCIUM DYNAMICS

(71) Applicant: Jenny YANG, Marietta, GA (US)

(72) Inventors: Jenny YANG, Marietta, GA (US); Xiaonan DENG, Atlanta, GA (US)

(21) Appl. No.: 18/685,995

(22) PCT Filed: Aug. 25, 2022
(86) PCT No.: PCT/US2022/075461

§ 371 Date: Feb. 23, 2024

Related U.S. Application Data
(60) Provisional application No. 63/236,946, filed on Aug. 25, 2021.

(51) Int. Cl. G01N 21/64; C07K 14/435; G01N 33/84

(52) U.S. Cl. CPC G01N 21/6428; C07K 14/43595; G01N 21/6456; G01N 33/84; G01N 2333/43595

(57) ABSTRACT

The present disclosure relates to engineered protein metal ion sensors and methods of measuring metal ions. Disclosed herein are polypeptide metal ion sensors comprising engineered green-fluorescent polypeptides and engineered red-fluorescent polypeptides and methods of detecting metal ions. The polypeptide metal ion sensors disclosed herein can provide for ultrafast kinetics, larger absorption changes, and/or a greater fluorescence dynamic range.

### CROSS-REFERENCE TO RELATED APPLICATIONS

This application claims the benefit of U.S. Provisional Application No. 63/236,946, filed Aug. 25, 2021, which is expressly incorporated herein by reference in its entirety.

### FIELD

The present disclosure relates to engineered protein metal ion sensors and methods of their use.

### BACKGROUND

Spatiotemporal calcium (Ca²⁺) signaling plays an essential role in physiological and pathological processes, such as synaptic transmission among neurons, excitation-contraction (EC) coupling in the muscle, and immune responses, spanning a timescale that ranges from a few milliseconds to hours. Dysfunction of Ca²⁺ dynamics has been linked to numerous diseases, including neurodegenerative disorders and calcitropic diseases. One major method of analyzing physiological and pathological states relies on monitoring Ca²⁺ dynamics, which is coupled with multiple receptors, channels, pumps, and exchangers. Thus, there is a pressing need to report Ca²⁺ dynamics with rapid kinetics and sufficient sensitivity. The compositions and methods disclosed herein address these and other needs.

### SUMMARY

Disclosed herein are polypeptide metal ion sensors comprising engineered green-fluorescent polypeptides and engineered red-fluorescent polypeptides and methods of detecting metal ions. The polypeptide metal ion sensors disclosed herein can provide for ultrafast kinetics, larger absorption changes, and/or a greater fluorescence dynamic range.

Herein, the examples show a novel red Ca²⁺ indicator, R-CatchER, with ultrafast kinetics, and an improved green Ca²⁺ indicator G-CatchER2 with larger absorption and fluorescence changes than previously reported designed green calcium sensors, which provide for the development of genetically encoded calcium indicators (GECIs) by tuning both protein properties and the electrostatic potential of the scaffold fluorescent proteins.

In some aspects, disclosed herein is a polypeptide metal ion sensor comprising an engineered green-fluorescent polypeptide having a heterologous metal ion binding site, wherein said engineered green-fluorescent polypeptide is a variant amino acid sequence of SEQ ID NO: 7 having the amino acid substitutions corresponding to S30R, Y39N, S147D, S175G, S202D, Q204E, F223E, and T225E and, when having a metal ion species bound thereto, exhibits an elevated fluorescence output compared to the polypeptide SEQ ID NO: 7 when binding to the same metal ion species.

In some embodiments, the amino acid sequence of said engineered green-fluorescent polypeptide comprises a sequence having about 95% similarity to SEQ ID NO: 10. In some embodiments, the amino acid sequence of said engineered green-fluorescent polypeptide comprises SEQ ID NO: 10. In some embodiments, the amino acid sequence of said engineered green-fluorescent polypeptide comprises a sequence having about 95% similarity to SEQ ID NO: 1-4, 7, 9-10, 13, or 17-22. In some embodiments, the amino acid sequence of said engineered green-fluorescent polypeptide comprises SEQ ID NO: 1-4, 7, 9-10, 13, or 17-22.

In some embodiments, said sensor comprises at least one targeting moiety that specifically recognizes a structural feature of a cell or tissue, or a target biomolecule.

In some embodiments, said at least one targeting moiety specifically recognizes a target component of an endoplasmic reticulum or a sarcoplasmic reticulum of a cell. In some embodiments, the endoplasmic reticulum-targeting moiety comprises a sequence about 95% identical to SEQ ID NO: 15 or 16. In some embodiments, the endoplasmic reticulum-targeting moiety comprises SEQ ID NO: 15 and 16.

In some embodiments, said at least one targeting moiety specifically recognizes a target component of a mitochondrion of a cell. In some embodiments, the targeting moiety comprises a sequence about 95% identical to SEQ ID NO: 33 or 34. In some embodiments, the targeting moiety comprises a sequence about 95% identical to SEQ ID NO: 33 and 34.

In some embodiments, said at least one targeting moiety specifically recognizes a target component of a subcellular environment of a cell including adjacent of channels and receptors. In some embodiments, said at least one targeting moiety specifically recognizes a transient receptor potential (TRP) channel, an N-methyl-D-aspartate (NMDA) receptor, and an α-amino-3-hydroxy-5-methyl-4-isoxazolepropionic acid (AMPA) receptor. In some embodiments, the targeting moiety comprises a sequence about 95% identical to SEQ ID NO: 38, 40, 42, 44, or 46.

In some embodiments, said at least one targeting moiety specifically recognizes a target polypeptide.

In some embodiments, said metal ion binding site specifically binds to a metal ion, wherein the metal is a lanthanide metal, an alkaline earth metal, lead, cadmium, or a transition metal. In some embodiments, the lanthanide metal is selected from the group consisting of lanthanum, gadolinium, and terbium. In some embodiments, the alkaline earth metal is selected from the group consisting of calcium, strontium, and magnesium. In some embodiments, the transition metal is selected from the group consisting of zinc and manganese.

In some aspects, disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 7 and having the amino acid substitutions corresponding to S30R, Y39N, S147D, S175G, S202D, Q204E, F223E, and T225E and, when having a metal ion species bound thereto, exhibits an elevated fluorescence output compared to the polypeptide sequence SEQ ID NO: 7 binding to the same metal ion species; (ii) delivering the polypeptide metal ion sensor, or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals.

In some embodiments, a detectable change in at least one of a wavelength, an intensity, and/or lifetime between the first and second spectroscopic signals indicates a change in the rate of release or intracellular concentration of a metal ion in the sample.

In some embodiments, the amino acid sequence of said engineered green-fluorescent polypeptide comprises a sequence having about 95% similarity to SEQ ID NO: 10.

In some embodiments, the amino acid sequence of said engineered green-fluorescent polypeptide comprises SEQ ID NO: 10.

In some embodiments, the amino acid sequence of said engineered green-fluorescent polypeptide comprises a sequence having about 95% similarity to SEQ ID NO: 1-4, 7, 9-10, 13, or 17-22. In some embodiments, the amino acid sequence of said engineered green-fluorescent polypeptide comprises SEQ ID NO: 1-4, 7, 9-10, 13, or 17-22.

In some embodiments, the detectable change in the signal intensity provides a quantitative measurement of the metal ion in the sample.

In some embodiments, the biological sample is a cell or tissue of an animal or human subject, or a cell or tissue isolated from an animal or human subject. In some embodiments, the spectroscopic signal generated when a metal ion is bound to said sensor is used to generate an image.

In some aspects, a polypeptide metal ion sensor comprising an engineered red-fluorescent polypeptide having a heterologous metal ion binding site, wherein said engineered red-fluorescent polypeptide is a variant amino acid sequence of SEQ ID NO: 11 having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species.

In some embodiments, the amino acid sequence of said engineered red-fluorescent polypeptide comprises a sequence having about 95% similarity to SEQ ID NO: 12. In some embodiments, the amino acid sequence of said engineered red-fluorescent polypeptide comprises SEQ ID NO: 12.

In some embodiments, the amino acid sequence of said engineered red-fluorescent polypeptide comprises a sequence having about 95% similarity to SEQ ID NO: 14, or 23-30. In some embodiments, the amino acid sequence of said engineered red-fluorescent polypeptide comprises SEQ ID NO: 14, or 23-30.

In some embodiments, said sensor comprises at least one targeting moiety that specifically recognizes a structural feature of a cell or tissue, or a target biomolecule. In some embodiments, said at least one targeting moiety specifically recognizes a target component of an endoplasmic reticulum or a sarcoplasmic reticulum of a cell. In some embodiments, the endoplasmic reticulum-targeting moiety comprises a sequence about 95% identical to SEQ ID NO: 15 or 16. In some embodiments, the endoplasmic reticulum-targeting moiety comprises SEQ ID NO: 15 and 16.

In some embodiments, said at least one targeting moiety specifically recognizes a target component of a mitochondrion of a cell. In some embodiments, the targeting moiety comprises a sequence about 95% identical to SEQ ID NO: 33 or 34. In some embodiments, the targeting moiety comprises SEQ ID NO: 33 and 34.

In some embodiments, said at least one targeting moiety specifically recognizes a target component of a subcellular environment of a cell including adjacent of channels and receptors. In some embodiments, said at least one targeting moiety specifically recognizes a transient receptor potential (TRP) channel, an N-methyl-D-aspartate (NMDA) receptor, and an α-amino-3-hydroxy-5-methyl-4-isoxazolepropionic acid (AMPA) receptor. In some embodiments, the targeting moiety comprises a sequence about 95% identical to SEQ ID NO: 38, 40, 42, 44, or 46.

In some embodiments, said at least one targeting moiety specifically recognizes a target polypeptide.

In some embodiments, said metal ion binding site specifically binds to a metal ion, wherein the metal is a lanthanide metal, an alkaline earth metal, lead, cadmium, or a transition metal. In some embodiments, the lanthanide metal is selected from the group consisting of lanthanum, gadolinium, and terbium. In some embodiments, the alkaline earth metal is selected from the group consisting of calcium, strontium, and magnesium. In some embodiments, the transition metal is selected from the group consisting of zinc and manganese.

In some aspects, disclosed herein is a method of detecting metal ions in a biological sample, comprising: (i) providing a polypeptide metal ion sensor comprising an engineered red-fluorescent polypeptide having a heterologous metal ion binding site, wherein said engineered red-fluorescent polypeptide is a variant of amino acid sequence SEQ ID NO: 11 and having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species; (ii) delivering the polypeptide metal ion sensor or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals.

In some embodiments, a detectable change in at least one of a wavelength, an intensity, and/or lifetime between the first and second spectroscopic signals indicates a change in the rate of release or intracellular concentration of a metal ion in the sample.

In some embodiments, the amino acid sequence of said engineered red-fluorescent polypeptide comprises a sequence having about 95% similarity to SEQ ID NO: 12. In some embodiments, the amino acid sequence of said engineered red-fluorescent polypeptide comprises SEQ ID NO: 12.

In some embodiments, the amino acid sequence of said engineered red-fluorescent polypeptide comprises a sequence having about 95% similarity to SEQ ID NO: 14, or 23-30. In some embodiments, the amino acid sequence of said engineered red-fluorescent polypeptide comprises SEQ ID NO: 14, or 23-30.

In some embodiments, the detectable change in the signal intensity provides a quantitative measurement of the metal ion in the sample.

In some embodiments, the biological sample is a cell or tissue of an animal or human subject, or a cell or tissue isolated from an animal or human subject.

In some embodiments, the spectroscopic signal generated when a metal ion is bound to said sensor is used to generate an image.

In some embodiments, the method of any preceding aspect further comprises a step of delivering to the biological sample a second polypeptide metal ion sensor comprising an engineered green-fluorescent polypeptide having a heterologous metal ion binding site, wherein said metal ion binding site specifically binds to a metal ion in the cytosol of the biological sample. In some embodiments, the second polypeptide metal ion sensor is a calmodulin-based sensor. In some embodiments, the second polypeptide metal ion sensor is jGCaMP7.

Also disclosed herein is a polypeptide metal ion sensor comprising an engineered red-fluorescent polypeptide having a heterologous metal ion binding site, wherein said engineered red-fluorescent polypeptide is a variant amino acid sequence of SEQ ID NO: 11 having the amino acid substitutions corresponding to A145E, K198D, and/or R216E and an amino acid substitution at residue K163. In some embodiments, wherein the amino acid substitute at residue K163 is K163Q, K163M, or K163L. In some embodiments, the polypeptide metal ion sensor further comprises a mitochondria targeting sequence.

### DESCRIPTION OF DRAWINGS

The accompanying figures, which are incorporated in and constitute a part of this specification, illustrate aspects described below.

FIG. 1 shows multiple time scales of protein motions and Ca2+ dynamics mediated by various receptors and biological functions.

FIGS. 2A-2B show rational design of GECIs with a single Ca²⁺-binding site by tuning protein properties. FIG. 2A shows normalized absorbance spectra of R-CatchER compared to mApple. FIG. 2B shows normalized emission spectra of R-CatchER.

FIGS. 3A and 3B show normalized absorbance spectra (FIG. 3A) and normalized emission spectra (FIG. 3B) of G-CatchER2 compared to G-CatchER.

FIGS. 4A-4I show characterization of R-CatchER. FIG. 4A shows normalized stopped-flow fluorescence of Ca²⁺ disassociation kinetics of R-CatchER. FIG. 4B shows comparison of Ca²⁺ disassociation kinetics among R-CatchER, G-CatchER, G-CEPIA1er and R-CEPIA1er. FIG. 4C shows normalized stopped-flow fluorescence of Ca²⁺ association kinetics of R-CatchER. FIG. 4D shows comparison of Ca²⁺ association kinetics among R-CatchER, G-CatchER, G-CEPIA1er and R-CEPIA1er at 1 mM Ca²⁺. FIG. 4E shows confocal imaging of R-CatchER with ER-tracker green in Hela cells (Pearson's Coefficient of 0.83). FIG. 4F shows ER Ca²⁺ dynamics measured by R-CatchER in response to both 500 μM and 1 mM 4-cmc in C2C12 cells. FIGS. 4G-4I show comparison of ER Ca²⁺ oscillation kinetics measured by R-CatchER and R-CEPIA1er in response to 100 μM histamine in HeLa cells. Half rise time and half decay time of the first peak of ER Ca²⁺ oscillation kinetics are compared.

FIGS. 5A-5I show spatial-temporal ER Ca²⁺ resolution of R-CatchER in neurons. FIG. 5A shows representative image of different regions of neuron upon 50 stimuli using R-CatchER and jGCaMP7s. A ΔF/F image of R-CatchER was overlaid onto a raw jGCaMP7s images to illustrate changes in fine processes (left). FIG. 5B shows representative traces of ER Ca²⁺ overloading in different regions of neuron following 50 stimuli using R-CatchER. FIGS. 5C-5D show Ca²⁺ release from the ER grouped by regions in dissociated hippocampal neurons after applying 100 μM of DHPG using R-CatchER (One-way ANOVA, Tukey's multiple comparisons). FIG. 5E shows representative traces of R-CatchER fluorescence change as a function of number of stimuli. FIGS. 5F-5G show half rise time and half decay time of R-CatchER as a function of number of stimuli (N=9). FIG. 5H shows representative traces of R-CatchER and jGCaMP7s following 50 stimuli in the soma. FIG. 5I shows representative traces of different regions of neuron upon 50 stimuli using jGCaMP7s.

FIGS. 6A-6F show that R-CatchER monitors ER Ca²⁺ dynamics mediated by CaSR. FIG. 6A shows CaSR transient transfected HEK293 cells. Synchronized ER and cytosolic Ca²⁺ oscillation mediated by CaSR in the presence of stepwise concentration of Ca²⁺. Blow up figure shows oscillations under 3 mM and 4 mM extracellular Ca²⁺. FIG. 6B shows ER Ca²⁺ oscillation frequency using R-CatchER in the presence of stepwise concentration of extracellular Ca²⁺. FIG. 6C shows R-CatchER traces of CaSR with the application of allosteric modulators or mutations in response to 4 mM Ca²⁺. FIG. 6D shows frequency comparison of R-CatchER for CaSR with the application of allosteric modulators or mutations in response to 4 mM Ca²⁺. FIG. 6E shows EC₅₀ of CaSR response to extracellular Ca²⁺ with mutations using R-CatchER. FIG. 6F shows distinct ER Ca²⁺ oscillation measured in TT cells in the presence of 10 μM Cinacalcet.

FIGS. 7A-7B show in situ characterization of G-CatchER2. FIG. 7A shows representative imaging curve and fitting curve of Ca²⁺ binding affinity of G-CatchER2 in HeLa cells (N=14) with stepwise Ca²⁺ concentrations. FIG. 7B shows ER Ca²⁺ dynamics measured by G-CatchER2 in response to 1 mM 4-cmc in C2C12 cells (N=10).

FIGS. 8A-8C show in vitro characterization of R-CatchER. FIG. 8A shows apparent k_{d} of R-CatchER to Ca²⁺. FIG. 8B shows Job's Plot of R-CatchER to Ca²⁺. FIG. 8C shows fluorescence responses of R-CatchER to various physiological molecules.

FIGS. 9A-9J show Ca²⁺ association and disassociation kinetics. FIG. 9A shows normalized fluorescence intensity of Ca²⁺ disassociation kinetics of G-CEPIA1er. FIG. 9B shows normalized fluorescence intensity of Ca²⁺ disassociation kinetics of R-CEPIA1er. FIG. 9C shows normalized fluorescence intensity of Ca²⁺ association kinetics of G-CEPIA1er. FIG. 9D shows normalized fluorescence intensity of Ca²⁺ association kinetics of R-CEPIA1er. FIGS. 9E-9F show Ca²⁺ association kinetics data of R-CEPIA1er were fitted by double exponential equation. FIGS. 9G-9H shows normalized fluorescence intensity of Ca²⁺ association and disassociation kinetics of R-CatchER E145D E147D (K_{d}=1.52±0.11 mM, ΔF/F=3.23±0.01). It maintains ultrafast kinetics (kₒₙ≥1.3×10⁶ M⁻¹s⁻¹, k_{off}≥1.9×10³ s⁻¹) FIGS. 9I-9J show normalized fluorescence intensity of Ca²⁺ association and disassociation kinetics of MCD1.

FIGS. 10A-10G show in situ characterization of R-CatchER. FIG. 10A shows comparison of blocking ER Ca²⁺ refilling measured by R-CEPIA1 er, G-CEPIA1er and R-CatchER in response to 0 mM Ca²⁺ with 3 μM Tg in HeLa cells. FIG. 10B shows ER Ca²⁺ oscillation kinetics measured by G-CEPIA1er in response to 100 μM histamine in HeLa cells. FIG. 10C shows frequency of the first peak of ER Ca²⁺ oscillation kinetics is compared measured by R-CatchER or R-CEPIA1er in response to 100 μM histamine in HeLa cells. FIG. 10D shows ER Ca²⁺ dynamics measured by R-CatchER in response to 100 μM ATP in HeLa cells. FIG. 10E shows fluorescence photobleaching experiments of R-CatchER in C2C12 cells. FIG. 10F shows representative imaging and fitting curve of Ca²⁺ binding affinity of R-CatchER in HeLa cells (0.31±0.05 mM, N=9) with stepwise Ca²⁺ concentrations. FIG. 10G shows resting ER Ca²⁺ concentration in HEK293 cells and HeLa cells measured by R-CatchER, with 0.68±0.22 and 0.59±0.16 mM, respectively.

FIGS. 11A-11E show spatial-temporal performance of R-CatchER in neurons. FIG. 11A shows representative image of co-immunostaining with SERCA2, confirmed proper targeting of ER with R-CatchER in neurons. FIG. 11B shows dynamic range of R-CatchER as a function of number of stimuli. FIG. 11C shows representative traces of different regions of neuron upon 50 stimuli using jGCaMP7s. FIG. 11D shows comparison of time to peak between R-CatchER and jGCaMP7s as a function of number of stimuli. FIG. 11E shows correlation of the amplitude of R-CatchER and jGCaMP7s as a function of number of stimuli.

FIGS. 12A-12K show ER Ca²⁺ dynamics mediated by CaSR using R-CatchER. FIG. 12A shows EC₅₀ comparison for WT CaSR of cytosolic Ca²⁺ oscillation using Fura-2 and ER Ca²⁺ oscillation using R-CatchER in the presence of different concentration of extracellular Ca²⁺. FIG. 12B shows synchronized ER Ca²⁺ oscillation with cytosolic Ca²⁺ oscillation mediated by CaSR in the presence of 500 μM TNCA with stepwise concentration of Ca²⁺. FIG. 12C shows synchronized ER Ca²⁺ oscillation with cytosolic Ca²⁺ oscillation mediated by CaSR in the presence of 50 nM Cinacalcet with stepwise concentration of Ca²⁺. FIG. 12D shows synchronized ER and cytosolic Ca²⁺ oscillation mediated by CaSR in the presence of 50 nM NPS-2143 with stepwise concentration of Ca²⁺. FIG. 12E shows synchronized ER and cytosolic Ca²⁺ oscillation mediated by CaSR in the presence of 5 mM L-Phe with stepwise concentration of Ca²⁺. FIG. 12F shows EC₅₀ of CaSR to extracellular Ca²⁺ in the presence or absence of L-Phe to CaSR, measured by R-CatchER. FIGS. 12G-12I show that ER oscillation was altered by applying 10 mM Ca²⁺ with 20 μM Ionomycin (FIG. 12G) 3 μM Tg (FIG. 12H) and 100 μM 2-APB (FIG. 12I). FIG. 12J shows real-time R-CatchER response of E297K CaSR in the absence and presence of 500 μM TNCA with stepwise extracellular Ca²⁺ concentration. FIG. 12K shows EC₅₀ of extracellular Ca²⁺ to E297K CaSR in the absence and presence of 500 μM TNCA using Rura-2, compared to WT CaSR.

FIGS. 13A-13F show CaSR mediated ER Ca²⁺ dynamics by R-CatchER. HEK293 cells, transient transfected with CaSR. FIG. 13A shows two different Ca²⁺ oscillation patterns: transient oscillations induced by 5 mM L-Phe under 0.5 mM Ca²⁺ and sinusoidal oscillations under 5 mM Ca²⁺ with R-CatchER. FIGS. 13B-13C show blocking Ca²⁺ influx through L-type Ca²⁺ channel by La³⁺ (100 μM) not only diminished the Ca²⁺ transient oscillation induced by L-Phe, but also decreased overall Ca²⁺ release from the ER, indicated by area under the curve (AUC) from 19.54±1.08 (N=30; 5 mM L-Phe alone) to 14.35±1.41 (N=27; 100 μM La³⁺ plus 5 mM L-Phe; p=0.006). FIGS. 13D-13F show such contribution of Ca²⁺ influx also depended on the extracellular Ca²⁺ concentration. ER Ca²⁺ frequency significantly decreased under 100 μM La³⁺ plus 5 mM L-Phe with either 2 mM Ca²⁺ or 3 mM Ca²⁺, while ER Ca²⁺ frequency stayed unchanged under 100 M La³⁺ plus 5 mM L-Phe with either 4 mM Ca²⁺ or 5 mM Ca²⁺.

FIG. 14 shows genetically encoded calcium indicators (GECIs), R-CatchER and G-CatchER2, with ultrafast kinetics and large fluorescence dynamic ranges are introduced. R-CatchER was applied to sense multi-scale calcium dynamics in endoplasmic reticulum. A design principle of GECIs is proposed that features a tuning of rapid dynamics and electrostatic potentials of fluorescent proteins.

FIG. 15 shows sequence alignment of EGFP (SEQ ID NO: 1), CatchER (SEQ ID NO: 2), G-CatchER⁺ (SEQ ID NO: 3), and G-CatchER2 (SEQ ID NO: 4).

FIG. 16 shows sequence alignment of R-CatchER (SEQ ID NO: 6) and mApple (SEQ ID NO: 5).

FIG. 17 shows mitochondria Ca²⁺ dynamics measured by mApple A145E/K198D/R216E. Mitochondria Ca²⁺ dynamics measured by mApple A145E/K198D/R216E in response to 100 μM histamine in HeLa cells (N=3). Scale bar is 20 μm.

FIGS. 18A-18 show improved mApple based mitochondria Ca²⁺ indicator. FIG. 18A shows normalized emission spectra of mApple A145E/K163L/K198D/R216E, Fₘₐₓ/Fₘᵢₙ=2.59±0.06. FIG. 18B shows apparent k_{d} of mApple A145E/K163L/K198D/R216E to Ca²⁺, 54.3±9.6 μM. FIG. 18C shows mitochondria Ca²⁺ dynamics measured by mApple A145E/K163L/K198D/R216E in response to 100 μM Histamine in HeLa cells (N=12). Scale bar is 20 μm.

FIGS. 19A-19B show that G-CatchER⁺ can Monitor Neuron ER Ca²⁺ Dynamics. (FIG. 19A) 100 μM of DHPG was added to initiate the release of Ca²⁺ from the ER via mGluR1/5 activation in hippocampal neurons. (FIG. 19B) Corresponding bar graph of DHPG-induced Ca2+ release (in ΔF/F0) from the ER grouped by neuron regions. Error bars are ±SEM, *p=0.02, one-way ANOVA, Tukey's multiple comparisons. A significant difference was observed in the G-CatchER⁺ response between secondary branchpoints and secondary dendrites (−0.19±0.1 versus −0.02±0.01). This shows a selective barrier or filtering mechanism of mGluR-dependent ER Ca²⁺ release in distal dendrites of hippocampal neurons.

FIGS. 20A-20F show validation of G-CatchER⁺ in neurons. (FIG. 20A) 0.5 mM of 4-cmc was added to initiate a RyR-dependent release of Ca2+ from the ER in mouse primary hippocampal neurons. (FIG. 20B) Corresponding bar graph of 4-cmc activated Ca²⁺ release (in ΔF/F0) from the ER grouped by neuron regions. Error bars are ±SEM, *p=0.05, one-way ANOVA, Tukey's multiple comparisons. (FIG. 20C) Inhibition of SERCA with 50 μM of CPA initiated a release of Ca2+ from the ER. (FIG. 20D) Corresponding bar graph of CPA inhibited Ca²⁺ release (in ΔF/F0) from the ER grouped by neuron regions. Error bars are ±SEM, one-way ANOVA, Tukey's multiple comparisons. (FIGS. 20E and 20F) Traces and amplitude of G-CatchER⁺ in response to 50 μM ionomycin and 10 mM Ca²⁺ in hippocampal neurons.

FIGS. 21A-21F show quantitative measurement of G-CatchER⁺ in different cell lines following addition of stimulatory or inhibitory agents, FIG. 25A. Basal ER Ca²⁺ estimation using G-CatchER⁺ in different cell lines. FIGS. 21B-21F. Estimated absolute ER Ca²⁺ change in response to 4-cmc, CPA, ATP and histamine in different cell lines using G-CatchER.

FIGS. 22A-22B show design of ER calcium sensor based on red fluorescent protein, mApple.

FIG. 23 shows that calcium binding site increased protonated form and decreased deprotonated form of the chromophore.

FIG. 24 shows that calcium binding decrease protonated form and increase deprotonated form of the chromophore.

FIGS. 25A-25C show design of GECIs by creating a single Ca²⁺ binding site. FIG. 25A shows residues on the surface of mApple to be used to design a single Ca²⁺ binding site. FIGS. 25A-25C shows correlation of Ca²⁺ induced dynamic range, the ratio of the anionic state over neutral state of the chromophore, and Ca²⁺ binding affinity with the number of the negatively charged residues of mApple.

FIGS. 26A-26F show quantum yield and extinction coefficient curves of mApple and R-CatchER. Curves of fluorescence intensity over Absorbance intensity at different protein concentrations of mCherry (FIG. 26A), Apo form of R-CatchER (FIG. 26C), and Holo form of R-CatchER (FIG. 26D). Curves of absorbance at 587 nm over Absorbance intensity at denatured form 455 nm at different protein concentrations of mCherry (FIG. 26B), Apo form of R-CatchER (FIG. 26E), and Holo form of R-CatchER (FIG. 26F).

FIGS. 27A-27F show using mRuby as a scaffold to design Ca²⁺ indicators. Absorbance and fluorescence spectra of mRubyP142ER198DH216EV218E (FIGS. 27A and 27D), mRubyT144ER198DH216EV218E (FIGS. 27B and 27E), and mRubyT144ER198DH216DV218E (FIGS. 27C and 27F).

FIGS. 28A-28B show in situ rapid response of R-CatchER. FIG. 28A. ER Ca2+ dynamics comparison measured by R-CatchER in response to either 100 μM ATP or 30 μM ATP in HEK293 cells. E. Dual color imaging using both R-CatchER and Fluo-4 to monitor ER and cytosolic Ca2+ dynamics in response to 100 μM histamine in Hela cells. A representative dual color imaging is shown. Scale bar is 20 μm.

FIGS. 29A-29B show quantitative basal Ca²⁺ measurement of R-CatchER in different CaSR mutations and cell lines. FIG. 29A. Estimated absolute ER Ca²⁺ concentration in different CaSR mutations using R-CatchER. FIG. 29B. Estimated absolute ER Ca²⁺ concentration in different cell lines using R-CatchER.

FIGS. 30A-30B show mGluR5 mediated Ca²⁺ dynamics by R-CatchER. mGluR5 transient transfected HEK293T cells. Synchronized ER Ca²⁺ oscillation with cytosolic Ca²⁺ oscillation mediated by mGluR5 in the presence of an increasing concentration of L-Glutamine (L-Glu). Blow up figure shows such oscillations under 10 μM L-Glu.

FIG. 31 shows catch series sensors targeting Ca²⁺ micro/nanodomains. Catch sensors can be applied using Drosophila binary expression systems; cell selective promoters driving lentiviral/AAV vectors; DIO-AAV FLEX Catch variants compatible with CRE transgenic drivers for in vitro and in vivo analyses.

### DETAILED DESCRIPTION

Therefore, in some aspects, disclosed herein are polypeptide metal ion sensors and uses thereof for detecting metal ions in a sample.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of 20%, ±10%, ±5%, or 10% from the measurable value.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

The term “engineered polypeptide” as used herein refers to a polypeptide that has been designed to have a heterologous metal ion binding site. The term “engineered” as used herein refers to the generation of mutations in the amino acid sequence of a polypeptide sensor such as a fluorescent protein to introduce negatively charged amino acids that on folding of the polypeptide form a calcium binding site or, if not participating in the site, generate advantageous properties in the sensor not found in the non-mutated parent sensor. For example, but not intended to be limiting, such advantageous properties may be a change in the detectable wavelength of the emitted fluorescence, in the intensity of the fluorescent signal, the magnitude of the signal under elevated temperatures, the kinetics of the binding and dissociation of the metal ion analyte, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA occurs.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

“Fluorescent protein” refers to any protein capable of emitting light when excited with appropriate electromagnetic radiation. Fluorescent proteins include proteins having amino acid sequences that are either natural or engineered,

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property.

The term “heterologous metal ion binding site” as used herein refers to a metal ion-specific binding site of an engineered polypeptide and which is not found in the native or wild-type fluorescent protein. In some embodiments, while the native protein may attract metal ions under some conditions, a heterologous site within the context of the disclosure refers to the juxtaposition of substituted and non-native amino acid side-chains that can form a binding site not found in the wild-type.

The term “co-operative interaction” as used herein refers to changing a fluorescent signal of a fluorescent protein, the changing being generated by the binding of a metal ion such as calcium to a calcium-binding site and the result in the forming of new bonds with a chromophore site within the protein due to conformational changes of the protein.

The term “heterologous negatively-charged amino acid substitution” as used herein refers to negatively-charged amino acids not found in the same position in the native or wild-type protein.

The term “identity” or “similarity” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” or “sequence similarity” to another sequence means that, when aligned, that percentage of bases (or amino acids) are the same in comparing the two sequences. This alignment and the percent similarity or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “sensor” is defined as an analytical tool comprised of biological components that are used to detect the presence of target(s) and to generate a signal. The term “polypeptide metal ion sensor” as used herein refers to a polypeptide that includes a metal ion binding site generated by the interaction of negatively-charged amino acid side-chains and a metal ion. Advantageously, the sensor can bind to calcium, but the sensors of the disclosure can be capable of binding other ions, most advantageously divalent ions.

“Targeting moiety” refers to a peptide capable of specifically binding to a target. “Specifically binding”, “specifically binds”, and “specifically recognizes” refers to the strength of the binding interaction between two molecules. In some embodiments, specificity is characterized by a dissociation constant of 10⁴M⁻¹ to 10¹²M⁻¹.

As used herein, a “target”, “target biomolecule”, or “target cell” refers to a biomolecule or a cell that can be the focus of a therapeutic drug strategy, diagnostic assay, detection assay, or a combination thereof. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

The term “variant” as used herein refers to a polypeptide or polynucleotide that differs from a reference polypeptide or polynucleotide, but retains essential properties. A typical variant of a polypeptide differs in amino acid sequence from another, reference, polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions).

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +2 is preferred, those within ±1 are particularly preferred, and those within +0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within +2 is preferred, those within +1 are particularly preferred, and those within +0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

### Compositions and Methods

In some aspects, disclosed herein is a polypeptide metal ion sensor comprising an engineered green-fluorescent polypeptide having a heterologous metal ion binding site, wherein said engineered green-fluorescent polypeptide is a variant amino acid sequence of SEQ ID NO: 7 having the amino acid substitutions corresponding to S30R, Y39N, S147D, S175G, S202D, Q204E, F223E, and/or T225E and, when having a metal ion species bound thereto, exhibits an elevated fluorescence output compared to the polypeptide SEQ ID NO: 7 when binding to the same metal ion species.

In some aspects, disclosed herein is a polypeptide metal ion sensor comprising an engineered green-fluorescent polypeptide having a heterologous metal ion binding site, wherein said engineered green-fluorescent polypeptide is a variant amino acid sequence of SEQ ID NO: 7 having the amino acid substitution corresponding to S147D and, when having a metal ion species bound thereto, exhibits an elevated fluorescence output compared to the polypeptide SEQ ID NO: 7 when binding to the same metal ion species.

In some aspects, disclosed herein is a polypeptide metal ion sensor comprising an engineered green-fluorescent polypeptide having a heterologous metal ion binding site, wherein said engineered green-fluorescent polypeptide is a variant amino acid sequence of SEQ ID NO: 7 having the amino acid substitution corresponding to S147D and at least one (or more) of the amino acid substitutions corresponding to S30R, Y39N, S175G, S202D, Q204E, F223E, and/or T225E and, when having a metal ion species bound thereto, exhibits an elevated fluorescence output compared to the polypeptide SEQ ID NO: 7 when binding to the same metal ion species.

In some aspects, disclosed herein is a polypeptide metal ion sensor comprising an engineered green-fluorescent polypeptide having a heterologous metal ion binding site, wherein said engineered green-fluorescent polypeptide is a variant amino acid sequence of SEQ ID NO: 1 having the amino acid substitutions corresponding to S31R, Y40N, S148D, S176G, S203D, Q205E, F224E, and/or T226E and, when having a metal ion species bound thereto, exhibits an elevated fluorescence output compared to the polypeptide SEQ ID NO: 1 when binding to the same metal ion species.

In some aspects, disclosed herein is a polypeptide metal ion sensor comprising an engineered green-fluorescent polypeptide having a heterologous metal ion binding site, wherein said engineered green-fluorescent polypeptide is a variant amino acid sequence of SEQ ID NO: 9 having the amino acid substitution corresponding to E147D and, when having a metal ion species bound thereto, exhibits an elevated fluorescence output compared to the polypeptide SEQ ID NO: 9 when binding to the same metal ion species.

In some embodiments, said engineered green-fluorescent polypeptide is a variant amino acid sequence of SEQ ID NO: 7 having the amino acid substitutions corresponding to S30R, Y39N, S147D, S175G, S202D, Q204E, F223E, and T225E. In some embodiments, said engineered green-fluorescent polypeptide is a variant amino acid sequence of SEQ ID NO: 7 having the amino acid substitutions corresponding to S30R. In some embodiments, said engineered green-fluorescent polypeptide is a variant amino acid sequence of SEQ ID NO: 7 having the amino acid substitutions corresponding to S30R, and Y39N. In some embodiments, said engineered green-fluorescent polypeptide is a variant amino acid sequence of SEQ ID NO: 7 having the amino acid substitutions corresponding to S30R, Y39N, and S147D. In some embodiments, said engineered green-fluorescent polypeptide is a variant amino acid sequence of SEQ ID NO: 7 having the amino acid substitutions corresponding to S30R, Y39N, S147D, and S175G. In some embodiments, said engineered green-fluorescent polypeptide is a variant amino acid sequence of SEQ ID NO: 7 having the one or more amino acid substitutions selected from S30R, Y39N, S147D, S175G, S202D, Q204E, F223E, and T225E.

In some embodiments, said engineered green-fluorescent polypeptide is a variant amino acid sequence of SEQ ID NO: 1 having the amino acid substitutions corresponding to S31R, Y40N, S148D, S176G, S203D, Q205E, F224E, and/or T226E. In some embodiments, said engineered green-fluorescent polypeptide is a variant amino acid sequence of SEQ ID NO: 1 having the amino acid substitutions corresponding to S31R, Y40N, S148D, S176G, S203D, Q205E, F224E, and T226E. In some embodiments, said engineered green-fluorescent polypeptide is a variant amino acid sequence of SEQ ID NO: 1 having the one or more amino acid substitutions selected from S31R, Y40N, S148D, S176G, S203D, Q205E, F224E, and T226E.

The term “increased”, “increase”, or “elevated” as used herein generally means an increase by a statically significant amount; for the avoidance of any doubt, “elevated” or “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the reference level is the fluorescence output of the polypeptide SEQ ID NO: 1 or SEQ ID NO: 7 when binding to the same metal ion species.

In some embodiments, said engineered green-fluorescent polypeptide, when having a metal ion species bound thereto, has an elevated fluorescence output compared to the polypeptide SEQ ID NO: 1 or 7 binding to the same metal ion species at or near a normal physiological temperature (including, for example, at about 36.0° C., about 36.1° C., about 36.2° C., about 36.3° C., about 36.4° C., about 36.5° C., about 36.6° C., about 36.7° C., about 36.8° C., about 36.9° C., about 37.0° C., about 37.1° C., about 37.2° C., about 37.3° C. about 37.4° C., about 37.5° C., about 37.6° C., about 37.7° C., about 37.8° C., about 37.9° C., or about 38° C.). In some embodiments, said engineered green-fluorescent polypeptide, when having a metal ion species bound thereto, has an elevated fluorescence output compared to the polypeptide SEQ ID NO: 1 or 7 binding to the same metal ion species at about 37.0° C.

In some embodiments, the amino acid sequence of said engineered green-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 10.

In some embodiments, the amino acid sequence of said engineered green-fluorescent polypeptide comprises a sequence having at least about 95% similarity (at least about 95%, at least about 96%, at least about 97%, at least about 98%, or at least about 99%) to SEQ ID NO: 10. In some embodiments, the amino acid sequence of said engineered green-fluorescent polypeptide comprises SEQ ID NO: 10. In some embodiments, the amino acid sequence of said engineered green-fluorescent polypeptide consists of SEQ ID NO: 10.

In some embodiments, the amino acid sequence of said engineered green-fluorescent polypeptide comprises a sequence having about 95% similarity to SEQ ID NO: 1-4, 7, 9-10, 13, or 17-22. In some embodiments, the amino acid sequence of said engineered green-fluorescent polypeptide comprises SEQ ID NO: 1-4, 7, 9-10, 13, or 17-22.

In some embodiments, the amino acid sequence of said engineered green-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 4. In some embodiments, the amino acid sequence of said engineered green-fluorescent polypeptide comprises SEQ ID NO: 4.

In some embodiments, the amino acid sequence of said engineered green-fluorescent polypeptide comprises a sequence having at least about 95% (at least about 95%, at least about 96%, at least about 97%, at least about 98%, or at least about 99%) similarity to SEQ ID NO: 4. In some embodiments, the amino acid sequence of said engineered green-fluorescent polypeptide comprises SEQ ID NO: 4. In some embodiments, the amino acid sequence of said engineered green-fluorescent polypeptide consists of SEQ ID NO: 4.

In some embodiments, said sensor comprises at least one targeting moiety that specifically recognizes a structural feature of a cell or tissue, or a target biomolecule.

In some embodiments, said at least one targeting moiety specifically recognizes a target component of an endoplasmic reticulum or a sarcoplasmic reticulum of a cell.

In some embodiments, said at least one targeting moiety specifically recognizes a target component of a mitochondrion of a cell. In some embodiments, the targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 33. In some embodiments, the targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 34. In some embodiments, the targeting moiety comprises SEQ ID NO: 33 and 34.

In some embodiments, said at least one targeting moiety specifically recognizes a target component of a subcellular environment of a cell including adjacent of channels and receptors. In some embodiments, said at least one targeting moiety specifically recognizes a calcium sensing receptor (CaSR), a metabotropic glutamate receptor (mGluR), a transient receptor potential (TRP) channel, an N-methyl-D-aspartate (NMDA) receptor, or an α-amino-3-hydroxy-5-methyl-4-isoxazolepropionic acid (AMPA) receptor.

In some embodiments, the calcium sensing receptor (CaSR) targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 38.

In some embodiments, the metabotropic glutamate receptor (mGluR) targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 40.

In some embodiments, the TRP channel targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 42.

In some embodiments, the NMDA receptor targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 44.

In some embodiments, the AMPA receptor targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 46.

In some embodiments, said at least one targeting moiety specifically recognizes a target polypeptide. In some embodiments, the targeting moiety is a targeting polypeptide motif (for example, a targeting polypeptide motif of SEQ ID NO: 15 or SEQ ID NO: 16 that specifically recognizes a target component of an endoplasmic reticulum or a sarcoplasmic reticulum of a cell). In some embodiments, said targeting moiety (e.g., targeting polypeptide motif) has at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) sequence similarity with an amino acid sequence selected from the group consisting of the sequences SEQ ID NOs: 15 and 16. In some embodiments, said at least one targeting moiety specifically recognizes a target polypeptide. In some embodiments, said targeting moiety (e.g., targeting polypeptide motif) has at least about 90% sequence similarity with an amino acid sequence selected from the group consisting of the sequences SEQ ID NOs: 15 and 16.

In some embodiments, said metal ion binding site specifically binds to a metal ion, wherein the metal is a lanthanide metal, an alkaline earth metal, lead, cadmium, or a transition metal. In some embodiments, the lanthanide metal is selected from the group consisting of lanthanum, gadolinium, and terbium. In some embodiments, the alkaline earth metal is selected from the group consisting of calcium, strontium, and magnesium. In some embodiments, the transition metal is selected from the group consisting of zinc and manganese.

In some aspects, disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 7 and having the amino acid substitutions corresponding to S30R, Y39N, S147D, S175G, S202D, Q204E, F223E, and/or T225E and, when having a metal ion species bound thereto, exhibits an elevated fluorescence output compared to the polypeptide SEQ ID NO: 7 binding to the same metal ion species; (ii) delivering the polypeptide metal ion sensor, or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals. In some embodiments, said engineered green-fluorescent polypeptide is a variant amino acid sequence of SEQ ID NO: 7 having the amino acid substitutions corresponding to S30R. In some embodiments, said engineered green-fluorescent polypeptide is a variant amino acid sequence of SEQ ID NO: 7 having the amino acid substitutions corresponding to S30R, and Y39N. In some embodiments, said engineered green-fluorescent polypeptide is a variant amino acid sequence of SEQ ID NO: 7 having the amino acid substitutions corresponding to S30R, Y39N, and S147D. In some embodiments, said engineered green-fluorescent polypeptide is a variant amino acid sequence of SEQ ID NO: 7 having the amino acid substitutions corresponding to S30R, Y39N, S147D, and S175G. In some embodiments, said engineered green-fluorescent polypeptide is a variant amino acid sequence of SEQ ID NO: 7 having the one or more amino acid substitutions selected from S30R, Y39N, S147D, S175G, S202D, Q204E, F223E, and T225E.

In some aspects, disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 7 and having the amino acid substitution corresponding to S147D and, when having a metal ion species bound thereto, exhibits an elevated fluorescence output compared to the polypeptide SEQ ID NO: 7 binding to the same metal ion species; (ii) delivering the polypeptide metal ion sensor, or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals.

In some aspects, disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 7 and having the amino acid substitution corresponding to S147D and at least one (or more) of the amino acid substitutions corresponding to S30R, Y39N, S175G, S202D, Q204E, F223E, and/or T225E and, when having a metal ion species bound thereto, exhibits an elevated fluorescence output compared to the polypeptide SEQ ID NO: 7 binding to the same metal ion species; (ii) delivering the polypeptide metal ion sensor, or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals. In some embodiments, said engineered green-fluorescent polypeptide is a variant amino acid sequence of SEQ ID NO: 7 having the amino acid substitutions corresponding to S30R. In some embodiments, said engineered green-fluorescent polypeptide is a variant amino acid sequence of SEQ ID NO: 7 having the amino acid substitutions corresponding to S30R, and Y39N. In some embodiments, said engineered green-fluorescent polypeptide is a variant amino acid sequence of SEQ ID NO: 7 having the amino acid substitutions corresponding to S30R, Y39N, and S147D. In some embodiments, said engineered green-fluorescent polypeptide is a variant amino acid sequence of SEQ ID NO: 7 having the amino acid substitutions corresponding to S30R, Y39N, S147D, and S175G. In some embodiments, said engineered green-fluorescent polypeptide is a variant amino acid sequence of SEQ ID NO: 7 having the one or more amino acid substitutions selected from S30R, Y39N, S147D, S175G, S202D, Q204E, F223E, and T225E.

In some aspects, disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 9 and having the amino acid substitution corresponding to E147D and, when having a metal ion species bound thereto, exhibits an elevated fluorescence output compared to the polypeptide SEQ ID NO: 9 binding to the same metal ion species; (ii) delivering the polypeptide metal ion sensor, or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals.

In some aspects, disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 1 and having the amino acid substitutions corresponding to S31R, Y40N, S148D, S176G, S203D, Q205E, F224E, and/or T226E and, when having a metal ion species bound thereto, exhibits an elevated fluorescence output compared to the polypeptide SEQ ID NO: 1 binding to the same metal ion species; (ii) delivering the polypeptide metal ion sensor, or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals. In some embodiments, said engineered green-fluorescent polypeptide is a variant amino acid sequence of SEQ ID NO: 1 having the amino acid substitutions corresponding to S31R, Y40N, S148D, S176G, S203D, Q205E, F224E, and T226E. In some embodiments, said engineered green-fluorescent polypeptide is a variant amino acid sequence of SEQ ID NO: 1 having the one or more amino acid substitutions selected from S31R, Y40N, S148D, S176G, S203D, Q205E, F224E, and T226E.

In some embodiments, a detectable change in at least one of a wavelength, an intensity, and/or lifetime between the first and second spectroscopic signals indicates a change in the rate of release or intracellular concentration of a metal ion in the sample.

In some embodiments, the amino acid sequence of said engineered green-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 10.

In some embodiments, the amino acid sequence of said engineered green-fluorescent polypeptide comprises a sequence having about 95% similarity to SEQ ID NO: 10. In some embodiments, the amino acid sequence of said engineered green-fluorescent polypeptide comprises SEQ ID NO: 10. In some embodiments, the amino acid sequence of said engineered green-fluorescent polypeptide consists of SEQ ID NO: 10.

In some embodiments, the amino acid sequence of said engineered green-fluorescent polypeptide comprises a sequence having about 95% similarity to SEQ ID NO: 1-4, 7, 9-10, 13, or 17-22. In some embodiments, the amino acid sequence of said engineered green-fluorescent polypeptide comprises SEQ ID NO: 1-4, 7, 9-10, 13, or 17-22.

In some embodiments, the amino acid sequence of said engineered green-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 4.

In some embodiments, the amino acid sequence of said engineered green-fluorescent polypeptide comprises a sequence having about 95% similarity to SEQ ID NO: 4. In some embodiments, the amino acid sequence of said engineered green-fluorescent polypeptide comprises SEQ ID NO: 4. In some embodiments, the amino acid sequence of said engineered green-fluorescent polypeptide consists of SEQ ID NO: 4.

In some embodiments, the detectable change in the signal intensity provides a quantitative measurement of the metal ion in the sample.

In some embodiments, the biological sample is a cell or tissue of an animal or human subject, or a cell or tissue isolated from an animal or human subject.

In some embodiments, the spectroscopic signal generated when a metal ion is bound to said sensor is used to generate an image.

Also disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 7 and having the amino acid substitutions corresponding to S30R, Y39N, S147D, S175G, S202D, Q204E, F223E, and/or T225E and, when having a metal ion species bound thereto, exhibits an elevated fluorescence output compared to the polypeptide SEQ ID NO: 7 binding to the same metal ion species; (ii) delivering the polypeptide metal ion sensor, or an expression vector having a nucleic acid sequence encoding said metal sensor to a biological sample; (iii) detecting a first fluorescent signal emitted by said sensor; (iv) generating a physiological or cellular change in the biological sample; (v) detecting a second fluorescent signal emitted by said sensor after step (iii); and (vi) comparing the first and second fluorescent signals. In some embodiments, a detectable change in at least one of a wavelength, an intensity, and/or lifetime between the first and second fluorescent signals indicates a change in the rate of release or intracellular concentration of a metal ion in the sample. In some embodiments, said engineered green-fluorescent polypeptide is a variant amino acid sequence of SEQ ID NO: 7 having the amino acid substitutions corresponding to S30R. In some embodiments, said engineered green-fluorescent polypeptide is a variant amino acid sequence of SEQ ID NO: 7 having the amino acid substitutions corresponding to S30R, and Y39N. In some embodiments, said engineered green-fluorescent polypeptide is a variant amino acid sequence of SEQ ID NO: 7 having the amino acid substitutions corresponding to S30R, Y39N, and S147D. In some embodiments, said engineered green-fluorescent polypeptide is a variant amino acid sequence of SEQ ID NO: 7 having the amino acid substitutions corresponding to S30R, Y39N, S147D, and S175G. In some embodiments, said engineered green-fluorescent polypeptide is a variant amino acid sequence of SEQ ID NO: 7 having the one or more amino acid substitutions selected from S30R, Y39N, S147D, S175G, S202D, Q204E, F223E, and T225E.

Also disclosed herein is a method of detecting metal ions in a biological sample, comprising: (i) providing a polypeptide metal ion sensor comprising an engineered green-fluorescent polypeptide having a heterologous metal ion binding site, wherein said engineered green-fluorescent polypeptide is a variant of amino acid sequence SEQ ID NO: 1 and having the amino acid substitutions corresponding to S31R, Y40N, S148D, S176G, S203D, Q205E, F224E, and/or T226E and, when having a metal ion species bound thereto, exhibits an elevated fluorescence output compared to the polypeptide SEQ ID NO: 1 binding to the same metal ion species; (ii) delivering the polypeptide metal ion sensor, or an expression vector having a nucleic acid sequence encoding said metal sensor to a biological sample; (iii) detecting a first fluorescent signal emitted by said sensor; (iv) generating a physiological or cellular change in the biological sample; (v) detecting a second fluorescent signal emitted by said sensor after step (iii); and (vi) comparing the first and second fluorescent signals. In some embodiments, a detectable change in at least one of a wavelength, an intensity, and/or lifetime between the first and second fluorescent signals indicates a change in the rate of release or intracellular concentration of a metal ion in the sample. In some embodiments, said engineered green-fluorescent polypeptide is a variant amino acid sequence of SEQ ID NO: 1 having the amino acid substitutions corresponding to S31R, Y40N, S148D, S176G, S203D, Q205E, F224E, and T226E. In some embodiments, said engineered green-fluorescent polypeptide is a variant amino acid sequence of SEQ ID NO: 1 having the one or more amino acid substitutions selected from S31R, Y40N, S148D, S176G, S203D, Q205E, F224E, and T226E.

In some embodiments, the detectable change in the signal intensity provides a quantitative measurement of the metal ion in the sample.

In some embodiments, polypeptide ion sensor comprises at least one targeting moiety that specifically recognizes a structural feature of a cell or tissue, or a target biomolecule. In some embodiments, said at least one targeting moiety specifically recognizes a target component of an endoplasmic reticulum or a sarcoplasmic reticulum of a cell.

In some embodiments, said at least one targeting moiety specifically recognizes a target component of a mitochondrion of a cell. In some embodiments, the targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 33. In some embodiments, the targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 34. In some embodiments, the targeting moiety comprises SEQ ID NO: 33 and 34.

In some embodiments, said at least one targeting moiety specifically recognizes a target component of a subcellular environment of a cell including adjacent of channels and receptors. In some embodiments, said at least one targeting moiety specifically recognizes a calcium sensing receptor (CaSR), a metabotropic glutamate receptor (mGluR), a transient receptor potential (TRP) channel, an N-methyl-D-aspartate (NMDA) receptor, or an α-amino-3-hydroxy-5-methyl-4-isoxazolepropionic acid (AMPA) receptor.

In some embodiments, the calcium sensing receptor (CaSR) targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 38.

In some embodiments, the metabotropic glutamate receptor (mGluR) targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 40.

In some embodiments, the TRP channel targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 42.

In some embodiments, the NMDA receptor targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 44.

In some embodiments, the AMPA receptor targeting moiety comprises a sequence about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) identical to SEQ ID NO: 46.

In some embodiments, said at least one targeting moiety specifically recognizes a target polypeptide. In some embodiments, the targeting moiety is a targeting polypeptide motif (for example, a targeting polypeptide motif of SEQ ID NO: 15 or SEQ ID NO: 16 that specifically recognizes a target component of an endoplasmic reticulum or a sarcoplasmic reticulum of a cell). In some embodiments, said targeting moiety (e.g., targeting polypeptide motif) has at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) sequence similarity with an amino acid sequence selected from the group consisting of the sequences SEQ ID NOs: 15 and 16. In some embodiments, said at least one targeting moiety specifically recognizes a target polypeptide. In some embodiments, said targeting moiety (e.g., targeting polypeptide motif) has at least about 90% sequence similarity with an amino acid sequence selected from the group consisting of the sequences SEQ ID NOs: 15 and 16.

In some aspects, disclosed herein is a polypeptide metal ion sensor comprising an engineered red-fluorescent polypeptide having a heterologous metal ion binding site, wherein said engineered red-fluorescent polypeptide is a variant amino acid sequence of SEQ ID NO: 11 having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species.

In some aspects, disclosed herein is a polypeptide metal ion sensor comprising an engineered red-fluorescent polypeptide having a heterologous metal ion binding site, wherein said engineered red-fluorescent polypeptide is a variant amino acid sequence of SEQ ID NO: 5 having the amino acid substitutions corresponding to A150E, K203D, and/or R221D and, when having a metal ion species bound thereto, exhibits an elevated fluorescence output compared to the polypeptide SEQ ID NO: 5 binding to the same metal ion species.

In some embodiments, said engineered red-fluorescent polypeptide is a variant amino acid sequence of SEQ ID NO: 11 having the amino acid substitutions corresponding to A145E, K198D, and R216D.

In some embodiments, said engineered red-fluorescent polypeptide is a variant amino acid sequence of SEQ ID NO: 5 having the amino acid substitutions corresponding to A150E, K203D, and R221D.

“Elevated” or “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the reference level is the fluorescence output of the polypeptide SEQ ID NO: 11 or SEQ ID NO: 5 when binding to the same metal ion species.

In some embodiments, said engineered red-fluorescent polypeptide, when having a metal ion species bound thereto, has an elevated fluorescence output compared to the polypeptide SEQ ID NO: 5 or SEQ ID NO: 11 binding to the same metal ion species at or near a normal physiological temperature (including, for example, at about 36.0° C., about 36.1° C., about 36.2° C., about 36.3° C., about 36.4° C., about 36.5° C., about 36.6° C., about 36.7° C., about 36.8° C., about 36.9° C., about 37.0° C., about 37.1° C., about 37.2° C., about 37.3° C. about 37.4° C., about 37.5° C., about 37.6° C., about 37.7° C., about 37.8° C., about 37.9° C., or about 38° C.). In some embodiments, said engineered red-fluorescent polypeptide, when having a metal ion species bound thereto, has an elevated fluorescence output compared to the polypeptide SEQ ID NO: 5 or SEQ ID NO: 11 binding to the same metal ion species at about 37.0° C.

In some embodiments, the engineered red-fluorescent polypeptide, having the amino acid substitutions A150E, K203D, and R221D relative to SEQ ID NO: 11, exhibits a faster fluorescence output compared to the polypeptide SEQ ID NO: 11 binding to the same metal ion species, for example, about at least about 20%, or at least about 30%, or at least about 40%, or at least about 50%, or at least about 60%, or at least about 70%, or at least about 80%, or at least about 90% or up to and including a 100% faster as compared to a reference level, or at least about a 2-fold, at least about a 3-fold, at least about a 4-fold, at least about a 5-fold, at least about a 10-fold, at least 100-fold, at least 1000-fold, at least 10,000 faster as compared to a reference level.

In some embodiments, the amino acid sequence of said engineered red-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 12.

In some embodiments, the amino acid sequence of said engineered red-fluorescent polypeptide comprises a sequence having at least about 95% similarity to SEQ ID NO: 12. In some embodiments, the amino acid sequence of said engineered red-fluorescent polypeptide comprises SEQ ID NO: 12. In some embodiments, the amino acid sequence of said engineered red-fluorescent polypeptide consists of SEQ ID NO: 12.

In some embodiments, the amino acid sequence of said engineered red-fluorescent polypeptide comprises a sequence having about 95% similarity to SEQ ID NO: 14, or 23-30. In some embodiments, the amino acid sequence of said engineered red-fluorescent polypeptide comprises SEQ ID NO: 14, or 23-30.

In some embodiments, the amino acid sequence of said engineered red-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 6.

In some embodiments, the amino acid sequence of said engineered red-fluorescent polypeptide comprises a sequence having at least about 95% similarity to SEQ ID NO: 6. In some embodiments, the amino acid sequence of said engineered red-fluorescent polypeptide comprises SEQ ID NO: 6. In some embodiments, the amino acid sequence of said engineered red-fluorescent polypeptide consists of SEQ ID NO: 6.

In some embodiments, said sensor comprises at least one targeting moiety that specifically recognizes a structural feature of a cell or tissue, or a target biomolecule.

In some embodiments, said at least one targeting moiety specifically recognizes a target component of an endoplasmic reticulum or a sarcoplasmic reticulum of a cell.

In some embodiments, said at least one targeting moiety specifically recognizes a target component of a mitochondrion of a cell. In some embodiments, the targeting moiety comprises a sequence about 95% identical to SEQ ID NO: 33 or 34. In some embodiments, the targeting moiety comprises a sequence about 95% identical to SEQ ID NO: 33 and 34.

In some embodiments, said at least one targeting moiety specifically recognizes a target component of a subcellular environment of a cell including adjacent of channels and receptors. In some embodiments, said at least one targeting moiety specifically recognizes a calcium sensing receptor (CaSR), a metabotropic glutamate receptor (mGluR), a transient receptor potential (TRP) channel, an N-methyl-D-aspartate (NMDA) receptor, or an α-amino-3-hydroxy-5-methyl-4-isoxazolepropionic acid (AMPA) receptor. In some embodiments, the targeting moiety comprises a sequence about 95% identical to SEQ ID NO: 38, 40, 42, 44, or 46.

In some embodiments, said at least one targeting moiety specifically recognizes a target polypeptide. In some embodiments, the targeting moiety is a targeting polypeptide motif (for example, a targeting polypeptide motif of SEQ ID NO: 15 or 16 that specifically recognizes a target component of an endoplasmic reticulum or a sarcoplasmic reticulum of a cell). In some embodiments, said targeting moiety (e.g., targeting polypeptide motif) has at least about 60% (for example, at least 60%, at least 65%, at least 70%, at least 75%, at least 80%, at least 85%, at least 90%, at least 95%, at least 96%, at least 97%, at least 98%, or at least 99%) sequence similarity with an amino acid sequence selected from the group consisting of the sequences SEQ ID NOs: 15 and 16. In some embodiments, said at least one targeting moiety (e.g., targeting polypeptide motif) specifically recognizes a target polypeptide. In some embodiments, said targeting moiety (e.g., targeting polypeptide motif) has at least about 90% sequence similarity with an amino acid sequence selected from the group consisting of the sequences SEQ ID NOs: 15 and 16.

In some embodiments, said metal ion binding site specifically binds to a metal ion, wherein the metal is a lanthanide metal, an alkaline earth metal, lead, cadmium, or a transition metal. In some embodiments, the lanthanide metal is selected from the group consisting of lanthanum, gadolinium, and terbium. In some embodiments, the alkaline earth metal is selected from the group consisting of calcium, strontium, and magnesium. In some embodiments, the transition metal is selected from the group consisting of zinc and manganese.

Also disclosed herein is a polypeptide metal ion sensor comprising an engineered red-fluorescent polypeptide having a heterologous metal ion binding site, wherein said engineered red-fluorescent polypeptide is a variant amino acid sequence of SEQ ID NO: 11 having the amino acid substitutions corresponding to A145E, K198D, and/or R216E and an amino acid substitution at residue K163. In some embodiments, wherein the amino acid substitute at residue K163 is K163Q, K163M, or K163L. In some embodiments, the polypeptide metal ion sensor further comprises a mitochondria targeting sequence.

In some aspects, disclosed herein is a method of detecting metal ions in a biological sample, comprising: (i) providing a polypeptide metal ion sensor comprising an engineered red-fluorescent polypeptide having a heterologous metal ion binding site, wherein said engineered red-fluorescent polypeptide is a variant of amino acid sequence SEQ ID NO: 11 and having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species; (ii) delivering the polypeptide metal ion sensor or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals.

In some aspects, disclosed herein is a method of detecting metal ions in a biological sample, comprising: (i) providing a polypeptide metal ion sensor comprising an engineered red-fluorescent polypeptide having a heterologous metal ion binding site, wherein said engineered red-fluorescent polypeptide is a variant of amino acid sequence SEQ ID NO: 5 and having the amino acid substitutions corresponding to A150E, K203D, and/or R221D and, when having a metal ion species bound thereto, exhibits an elevated fluorescence output compared to the polypeptide SEQ ID NO: 5 binding to the same metal ion species; (ii) delivering the polypeptide metal ion sensor or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals.

In some embodiments, a detectable change in at least one of a wavelength, an intensity, and/or lifetime between the first and second spectroscopic signals indicates a change in the rate of release or intracellular concentration of a metal ion in the sample.

In some embodiments, the amino acid sequence of said engineered red-fluorescent polypeptide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 12.

In some embodiments, the amino acid sequence of said engineered red-fluorescent polypeptide comprises a sequence having about 95% similarity to SEQ ID NO: 12. In some embodiments, the amino acid sequence of said engineered red-fluorescent polypeptide comprises SEQ ID NO: 12. In some embodiments, the amino acid sequence of said engineered red-fluorescent polypeptide consists of SEQ ID NO: 12.

In some embodiments, the amino acid sequence of said engineered red-fluorescent polypeptide comprises a sequence having about 95% similarity to SEQ ID NO: 14, or 23-30. In some embodiments, the amino acid sequence of said engineered red-fluorescent polypeptide comprises SEQ ID NO: 14, or 23-30.

In some embodiments, the amino acid sequence of said engineered red-fluorescent polypeptide comprises a sequence having at about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 6.

In some embodiments, the amino acid sequence of said engineered red-fluorescent polypeptide comprises a sequence having about 95% similarity to SEQ ID NO: 6. In some embodiments, the amino acid sequence of said engineered red-fluorescent polypeptide comprises SEQ ID NO: 6. In some embodiments, the amino acid sequence of said engineered red-fluorescent polypeptide consists of SEQ ID NO: 6.

In some embodiments, the detectable change in the signal intensity provides a quantitative measurement of the metal ion in the sample.

In some embodiments, the biological sample is a cell or tissue of an animal or human subject, or a cell or tissue isolated from an animal or human subject.

In some embodiments, the spectroscopic signal generated when a metal ion is bound to said sensor is used to generate an image. In some embodiments, the spectroscopic signal is a fluorescent signal. In some embodiments, the spectroscopic signal is an absorbance signal.

In some embodiments, polypeptide ion sensor comprises at least one targeting moiety that specifically recognizes a structural feature of a cell or tissue, or a target biomolecule. In some embodiments, said at least one targeting moiety specifically recognizes a target component of an endoplasmic reticulum or a sarcoplasmic reticulum of a cell.

In some embodiments, said at least one targeting moiety specifically recognizes a target component of a mitochondrion of a cell. In some embodiments, the targeting moiety comprises a sequence about 95% identical to SEQ ID NO: 33 or 34. In some embodiments, the targeting moiety comprises a sequence about 95% identical to SEQ ID NO: 33 and 34.

In some embodiments, said at least one targeting moiety specifically recognizes a target component of a subcellular environment of a cell including adjacent of channels and receptors. In some embodiments, said at least one targeting moiety specifically recognizes a calcium sensing receptor (CaSR), a metabotropic glutamate receptor (mGluR), a transient receptor potential (TRP) channel, an N-methyl-D-aspartate (NMDA) receptor, or an α-amino-3-hydroxy-5-methyl-4-isoxazolepropionic acid (AMPA) receptor. In some embodiments, the targeting moiety comprises a sequence about 95% identical to SEQ ID NO: 38, 40, 42, 44, or 46.

In some embodiments, said at least one targeting moiety specifically recognizes a target polypeptide. In some embodiments, the targeting moiety is a targeting polypeptide motif (for example, a targeting polypeptide motif of SEQ ID NO: 15 or 16 that specifically recognizes a target component of an endoplasmic reticulum or a sarcoplasmic reticulum of a cell). In some embodiments, said targeting moiety (e.g., targeting polypeptide motif) has at least about 60% (for example, at least 60%, at least 65%, at least 70%, at least 75%, at least 80%, at least 85%, at least 90%, at least 95%, at least 96%, at least 97%, at least 98%, or at least 99%) sequence similarity with an amino acid sequence selected from the group consisting of the sequences SEQ ID NOs: 15 and 16. In some embodiments, said at least one targeting moiety (e.g., targeting polypeptide motif) specifically recognizes a target polypeptide. In some embodiments, said targeting moiety (e.g., targeting polypeptide motif) has at least about 90% sequence similarity with an amino acid sequence selected from the group consisting of the sequences SEQ ID NOs: 15 and 16.

In some examples, the method disclosed herein detecting metal ions in different cellular compartments (e.g., cytosol versus ER, mitochondria, a channel, or a receptor). In some embodiments, the method of any preceding aspect further comprises a step of delivering to the biological sample a second polypeptide metal ion sensor comprising an engineered green-fluorescent polypeptide having a heterologous metal ion binding site, wherein said metal ion binding site specifically binds to a metal ion in the cytosol of the biological sample. In some embodiments, the second polypeptide metal ion sensor is a calmodulin-based sensor. In some embodiments, the second polypeptide metal ion sensor is jGCaMP7.

Accordingly, in some aspects, disclosed herein is a method of detecting metal ions in a biological sample, comprising: (i) providing a first polypeptide metal ion sensor comprising an engineered red-fluorescent polypeptide having a heterologous metal ion binding site, wherein said engineered red-fluorescent polypeptide is a variant of amino acid sequence SEQ ID NO: 11 and having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species; (ii) delivering the first polypeptide metal ion sensor or an expression vector having a nucleic acid sequence encoding said metal sensor to a biological sample; (iii) detecting a first spectroscopic signal emitted by said sensor; (iv) generating a physiological or cellular change in the biological sample; (v) detecting a second spectroscopic signal emitted by said sensor after step (iii); and (vi) comparing the first and second spectroscopic signals, wherein the method further comprises delivering to the biological sample a second polypeptide metal ion sensor comprising an engineered green-fluorescent polypeptide having a heterologous metal ion binding site, wherein said metal ion binding site specifically binds to a metal ion in the cytosol of the biological sample. In some embodiments, the second polypeptide metal ion sensor is a calmodulin-based sensor. In some embodiments, the second polypeptide metal ion sensor is jGCaMP7.

Also disclosed herein is a method of detecting metal ions in a biological sample, comprising: (i) providing a polypeptide metal ion sensor comprising an engineered red-fluorescent polypeptide having a heterologous metal ion binding site, wherein said engineered red-fluorescent polypeptide is a variant of amino acid sequence SEQ ID NO: 11 and having the amino acid substitutions corresponding to A145E, K198D, and/or R216D and, when having a metal ion species bound thereto, has an elevated fluorescence output compared to the polypeptide SEQ ID NO: 11 binding to the same metal ion species; (ii) delivering the polypeptide metal ion sensor, or an expression vector having a nucleic acid sequence encoding said metal sensor to a biological sample; (iii) detecting a first fluorescent signal emitted by said sensor; (iv) generating a physiological or cellular change in the biological sample; (v) detecting a second fluorescent signal emitted by said sensor after step (iii); and (vi) comparing the first and second fluorescent signals. In some embodiments, a detectable change in at least one of a wavelength, an intensity, and/or lifetime between the first and second fluorescent signals indicates a change in the rate of release or intracellular concentration of a metal ion in the sample.

Also disclosed herein is a method of detecting metal ions in a biological sample, comprising: (i) providing a polypeptide metal ion sensor comprising an engineered red-fluorescent polypeptide having a heterologous metal ion binding site, wherein said engineered red-fluorescent polypeptide is a variant of amino acid sequence SEQ ID NO: 5 and having the amino acid substitutions corresponding to A150E, K203D, and/or R221D and, when having a metal ion species bound thereto, has an elevated fluorescence output compared to the polypeptide SEQ ID NO: 5 binding to the same metal ion species; (ii) delivering the polypeptide metal ion sensor, or an expression vector having a nucleic acid sequence encoding said metal sensor to a biological sample; (iii) detecting a first fluorescent signal emitted by said sensor; (iv) generating a physiological or cellular change in the biological sample; (v) detecting a second fluorescent signal emitted by said sensor after step (iii); and (vi) comparing the first and second fluorescent signals. In some embodiments, a detectable change in at least one of a wavelength, an intensity, and/or lifetime between the first and second fluorescent signals indicates a change in the rate of release or intracellular concentration of a metal ion in the sample.

In some embodiments, the detectable change in the signal intensity provides a quantitative measurement of the metal ion in the sample.

Also disclosed herein is a recombinant polynucleotide that encodes the metal ion sensor comprising an engineered green-fluorescent polypeptide disclosed herein. In some embodiments, the recombinant polynucleotide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 13.

Also disclosed herein is a recombinant polynucleotide that encodes the metal ion sensor comprising an engineered red-fluorescent polypeptide disclosed herein. In some embodiments, the recombinant polynucleotide comprises a sequence having at least about 60% (for example, at least about 60%, at least about 65%, at least about 70%, at least about 75%, at least about 80%, at least about 85%, at least about 90%, at least about 95%, at least about 96%, at least about 97%, at least about 98%, or at least about 99%) similarity to SEQ ID NO: 14.

Also disclosed herein is a vector comprising the recombinant polynucleotide disclosed herein.

Also disclosed herein is a method of diagnosing a calcium-sensing receptor-related disorder in a subject in need, comprising (i) obtaining a biological sample from the subject; (ii) delivering to the biological sample the polypeptide metal ion sensor, or an expression vector having a nucleic acid sequence encoding said metal sensor of any preceding aspect; and (iii) detecting a frequency of calcium oscillation in the biological sample; wherein a decreased frequency of calcium oscillation as compared to a reference control is indicative of the subject having the calcium-sensing receptor-related disorder. In some embodiments, the polypeptide metal ion sensor comprises an engineered green-fluorescent polypeptide having a heterologous metal ion binding site, wherein said engineered green-fluorescent polypeptide is a variant of amino acid sequence SEQ ID NO: 7 and has the amino acid substitutions corresponding to S30R, Y39N, S147D, S175G, S202D, Q204E, F223E, and T225E. In some embodiments, the polypeptide metal ion sensor comprises an engineered red-fluorescent polypeptide having a heterologous metal ion binding site, wherein said engineered red-fluorescent polypeptide is a variant of amino acid sequence SEQ ID NO: 11 and has the amino acid substitutions corresponding to A145E, K198D, and/or R216D. In some embodiments, the polypeptide metal ion sensor further comprises at least one targeting moiety that specifically recognizes a structural feature of a cell or tissue, or a target biomolecule. In some embodiments, said at least one targeting moiety specifically recognizes a target component of an endoplasmic reticulum or a sarcoplasmic reticulum of a cell. In some embodiments, said at least one targeting moiety specifically recognizes a target component of a mitochondrion of a cell. In some embodiments, the targeting moiety comprises a sequence about 95% identical to SEQ ID NO: 33. In some embodiments, said at least one targeting moiety specifically recognizes a target component of a subcellular environment of a cell including adjacent of channels and receptors. In some embodiments, said at least one targeting moiety specifically recognizes a calcium sensing receptor (CaSR), a metabotropic glutamate receptor (mGluR), a transient receptor potential (TRP) channel, an N-methyl-D-aspartate (NMDA) receptor, or an α-amino-3-hydroxy-5-methyl-4-isoxazolepropionic acid (AMPA) receptor.

Also disclosed herein is a method of screening drugs for treatment of a calcium-sensing receptor-related disorder, comprising (i) obtaining a plurality of cells having a mutated Ca2+-sensing receptor (CaSR); (ii) applying a drug to the cells; (iii) delivering to the cells the polypeptide metal ion sensor, or an expression vector having a nucleic acid sequence encoding said metal sensor of any preceding aspect; and (iv) detecting a frequency of calcium oscillation in the cells; wherein an increased frequency of calcium oscillation of the cells as compared to a reference control indicates the drug as effective for treatment of the calcium-sensing receptor-related disorder. In some embodiments, the polypeptide metal ion sensor comprises an engineered green-fluorescent polypeptide having a heterologous metal ion binding site, wherein said engineered green-fluorescent polypeptide is a variant of amino acid sequence SEQ ID NO: 7 and has the amino acid substitutions corresponding to S30R, Y39N, S147D, S175G, S202D, Q204E, F223E, and T225E. In some embodiments, the polypeptide metal ion sensor comprises an engineered red-fluorescent polypeptide having a heterologous metal ion binding site, wherein said engineered red-fluorescent polypeptide is a variant of amino acid sequence SEQ ID NO: 11 and has the amino acid substitutions corresponding to A145E, K198D, and/or R216D. In some embodiments, the the polypeptide metal ion sensor further comprises at least one targeting moiety that specifically recognizes a structural feature of a cell or tissue, or a target biomolecule. In some embodiments, said at least one targeting moiety specifically recognizes a target component of an endoplasmic reticulum or a sarcoplasmic reticulum of a cell. In some embodiments, said at least one targeting moiety specifically recognizes a target component of a mitochondrion of a cell. In some embodiments, the targeting moiety comprises a sequence about 95% identical to SEQ ID NO: 33. In some embodiments, said at least one targeting moiety specifically recognizes a target component of a subcellular environment of a cell including adjacent of channels and receptors. In some embodiments, said at least one targeting moiety specifically recognizes a calcium sensing receptor (CaSR), a metabotropic glutamate receptor (mGluR), a transient receptor potential (TRP) channel, an N-methyl-D-aspartate (NMDA) receptor, or an α-amino-3-hydroxy-5-methyl-4-isoxazolepropionic acid (AMPA) receptor.

### EXAMPLES

The following examples are set forth below to illustrate the compositions, polypeptides, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Introduction

Tremendous efforts have been devoted to the development of genetically encoded Ca²⁺ indicators (GECIs), which have the advantage of genetic targeting over synthetic Ca²⁺ dyes. However, current GECIs are almost exclusively based on native Ca²⁺-binding proteins with multiple binding sites, such as calmodulin (CaM) and troponin C, and large-scale mutagenesis has been the primary approach to optimize Ca²⁺-binding affinities or fluorescence sensitivities. Moreover, these indicators rely on Ca²⁺-dependent binding of CaM to its targeted peptides, a rate-limiting step that undergoes conformational changes on a timescale of milliseconds. Mutations on the CaM binding peptide, RS20, were reported to improve in vitro kinetics, but the Ca²⁺ sensitivity is compromised with a significantly reduced dynamic range. Consequently, alternative strategies to rationally design Ca²⁺ indicators with a single Ca²⁺-binding site and rapid kinetics are urgently needed.

To fill in this gap, a green genetically encoded Ca²⁺ indicator, CatchER (later renamed as G-CatchER), was initially developed by creating a single Ca²⁺ binding site directly on the enhanced green fluorescent protein (EGFP) scaffold to alter the electrostatics near the chromophore. G-CatchER exhibited faster kinetics than conventional CaM based indicators, since it does not require large conformational changes upon Ca²⁺ binding. However, G-CatchER exhibited a relatively small Ca²⁺-induced fluorescence dynamic range, partially because the criteria used in developing G-CatchER prioritized the alteration of Ca²⁺ binding affinity, rather than the dynamic range, by optimizing the geometry of the Ca²⁺ binding site.

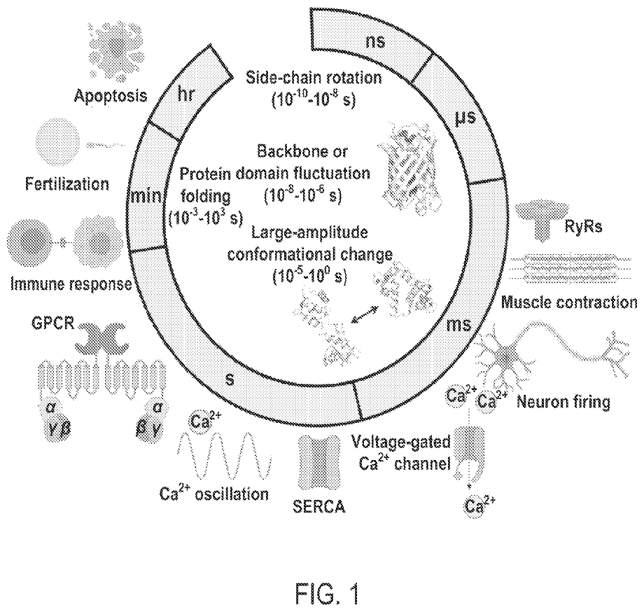

Comparable to Ca²⁺ dynamics, protein internal conformational dynamics occur across multiple spatiotemporal scales, from fast side-chain reorientations (ps-ns) and backbone fluctuations (ns-μs), to slow large-amplitude conformational changes (>10 μs) (FIG. 1). These motions reflect a protein populating multiple conformational substrates over a broad energy landscape, and these are coupled to protein functions, including enzyme catalysis, allosteric regulation, and protein-ligand recognitions, which underlie diverse cellular processes. Mutations, ligand binding (e.g., Ca²⁺-binding), and other perturbations modify the energy landscape, causing a shift in the conformational ensemble of the protein toward altered dynamics and functions. Understanding the relationship between protein dynamics and function may inform on new design strategies of GECIs.

Herein, the examples show a novel Ca²⁺ indicator, R-CatchER, with ultrafast kinetics and provide for the development of GECIs by tuning both protein dynamics and the electrostatic potential of the scaffold fluorescent proteins. To validate the principle, G-CatchER2 was developed, an improved version of G-CatchER that has a larger Ca²⁺-binding induced absorption change and exhibits a greater fluorescence dynamic range. The study also demonstrate the applications of these new indicators to reveal rapid Ca²⁺ dynamics in one key intracellular organelle, the endoplasmic reticulum (ER) of various cell types. R-CatchER enabled the first report of ER Ca²⁺ oscillations mediated by calcium sensing receptors (CaSRs) and revealed ER Ca²⁺-based functional cooperativity of CaSR.

### Example 2. Results

Designing Ca2+ indicators. CatchER was created by directly engineering a Ca²⁺-binding site on the surface of enhanced green fluorescent protein (EGFP). The binding site was made of residues 147, 149, 202, 204, 223, and 225 to form a hemispherical shape preferring Ca²⁺ binding. By site-directed mutagenesis of these residues to be either Glu or Asp, absorbance intensity ratio of anionic state (569 nm) over neutral state (455 nm) of the EGFP chromophore and fluorescence dynamic range after Ca²⁺ binding increased, with an increasing number of negatively charged residues of the binding site. In contrast, the Ca²⁺ binding affinity decreased. CatchER, with 5 negatively charged residues (S147E, S202D, Q204E, F223E, and T225E), exhibits highest the fluorescence dynamic range (ΔF/F=1.89±0.03) than other variants. Reversely, such ratio of anionic state over neutral state decreased when CatchER mixed with 10 mM Ca²⁺, indicating that binding to Ca²⁺ favors the anionic form of the chromophore.

Additionally, In CatchER, creating the Ca²⁺ binding site shifted the population between protonated and deprotonated states of the chromophore, and Ca²⁺ binding recovered such alteration. In mCherry variants, we did not observe such changes, suggesting that changing the pKa or the population between protonated and deprotonated states of the chromophore is necessary.

Therefore, several strategies were proposed herein to generally create Ca2+ indicators: 1) The equilibrium between the protonated and deprotonated forms of chromophore would be affected by introducing negatively charged residues, the more negatively charged residues, the more protonated form of chromophore over deprotonated. 2) Ca²⁺ binding to the protein would perturb the equilibrium, by stabilizing the deprotonated form of the chromophore. 3) Ca²⁺ binding to the protein would also rigidify the chromophore by increasing both quantum yield and extinction coefficient. 4) The apparent pKa of the chromophore decreased as introducing more negative charged residues. Whereas Ca²⁺ binding to the protein decreasing the pKa.

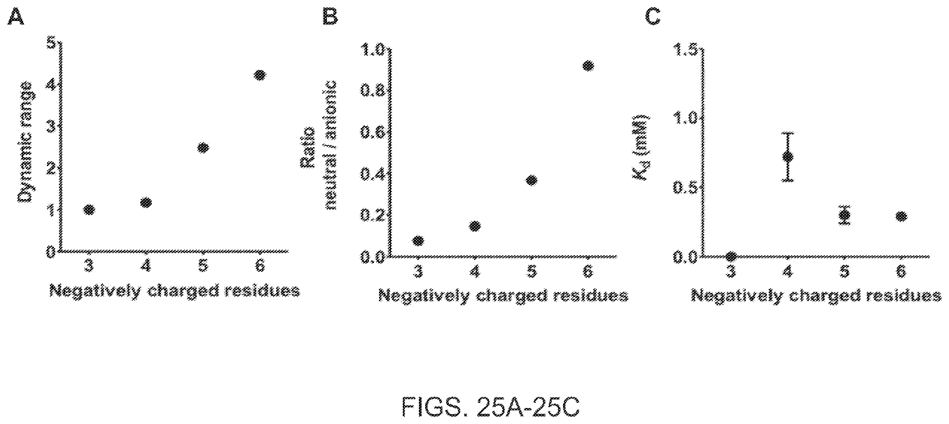

Develop ER Ca²⁺ indicator based on red fluorescent proteins, mApple and mRuby. To verify these strategies, red fluorescent proteins, mApple and mRuby, were chosen to create red color ER GECIs. As for mApple, residues 145, 147, 196, 198, 216, and 218 were used for the Ca²⁺ binding site. Similar position as CatchER, residues 145, 147, 196, 198, 216 and 218 of mApple were used for the Ca²⁺ binding site. Consistently, associated with the increasing number of the negatively charged residues, fluorescence dynamic range and ratio of anionic state over neutral state increased (FIG. 25). Notably, the increasing number of the negatively charged residues shifted the equilibrium between the protonated and deprotonated forms of the chromophore (FIG. 25). In contrast, Ca²⁺ binding affinity did not follow a certain trend.

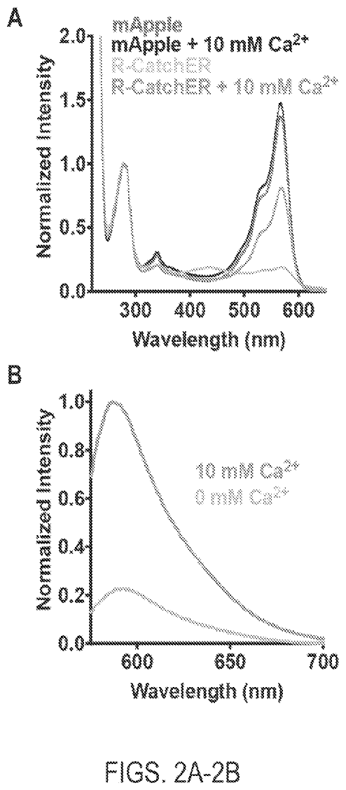

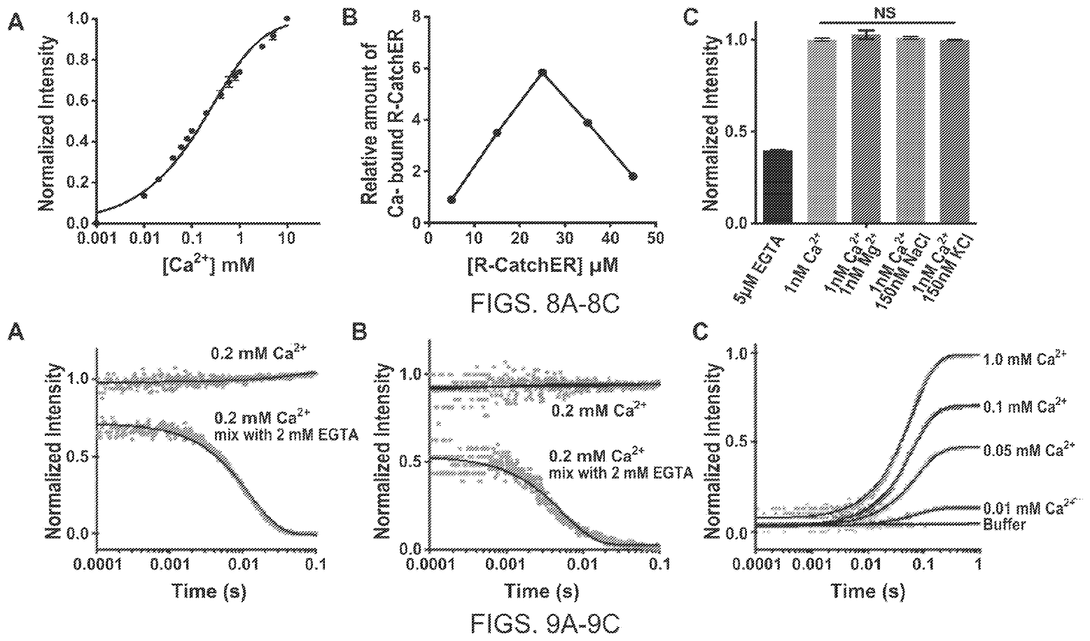

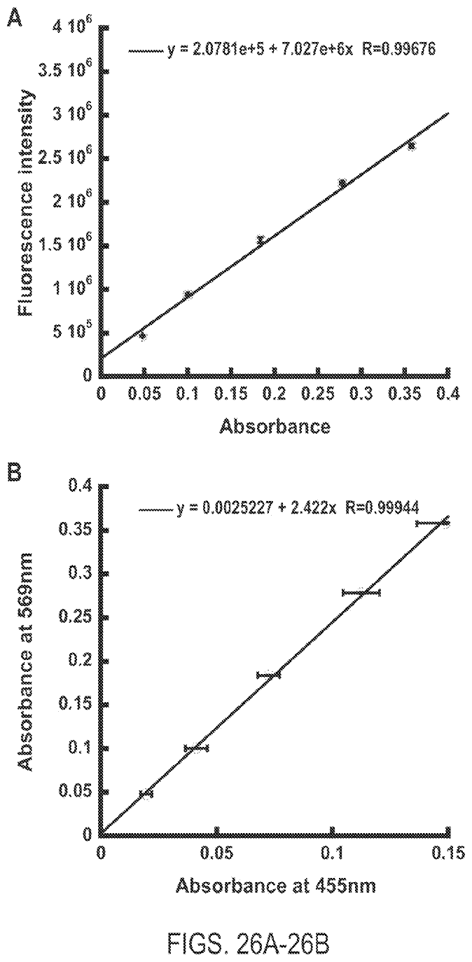

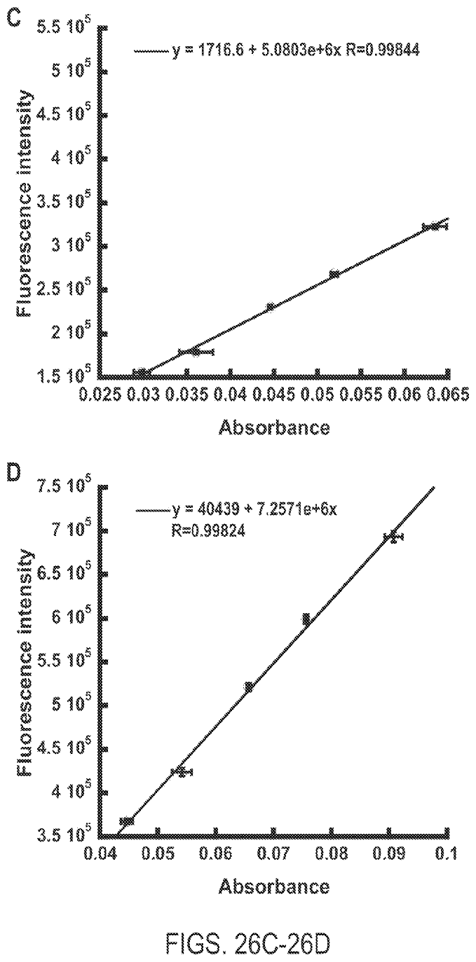

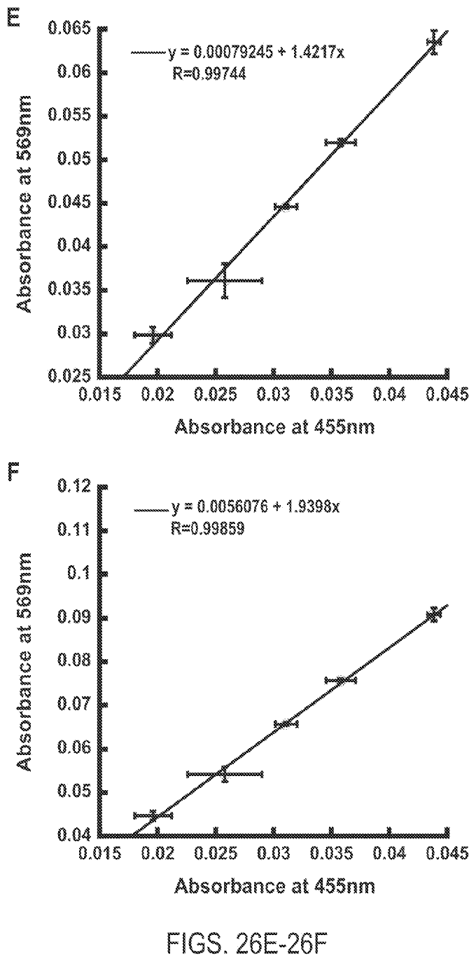

Significantly, 6 negatively charged (A145E, E147, D196, K198D, R216E, and E218), R-CatchER, shows a larger fluorescence dynamic range (ΔF/F=4.22±0.04) than other variants. Mixed with 10 mM Ca²⁺, a dramatic shift towards the anionic state of the chromophore was also observed (FIG. 2 and FIG. 8). R-CatchER was bacterial expressed and purified, and its optical properties were determined using UV spectrophotometer and fluorescence spectroscopy. Excited at 569 nm, fluorescence intensity increases of R-CatchER with different concentrations of Ca²⁺ were well fitted to a 1:1 binding equation. K_{d} value for Ca²⁺ binding is 0.35±0.03 mM. 1:1 stoichiometry of R-CatchER to Ca²⁺ was further validated using Job Plot (FIG. 2 and FIG. 8). Additionally, Ca²⁺ induced fluorescence changes were insensitive to the addition of 1 mM Mg²⁺, 150 mM KCl, and 150 mM NaCl, indicating that R-CatchER has preferential Ca²⁺ metal selectivity over other ions (FIG. 2 and FIG. 8). Ca²⁺ binding assisted chromophore formation of R-CatchER, as shown of apparent pKa of R-CatchER from 8.58±0.11 at 0 mM Ca²⁺ to 7.11±0.10 with additional 10 mM Ca²⁺ (FIG. 2 and FIG. 8). R-CatchER showed the highest pKa shift among other variants, suggesting the important roles of chromophore population shift resulting in Ca²⁺ fluorescence increase (Table. 6). After binding to Ca²⁺, fluorescence quantum yield and brightness of R-CatchER increased, which are comparable to its scaffold protein mApple, indicating a suitable imaging capacity of R-CatchER (Table. 6 and FIG. 26). Taken together, these results show R-CatchER binding to Ca²⁺ is involved with a concomitant recovery of fluorescence.

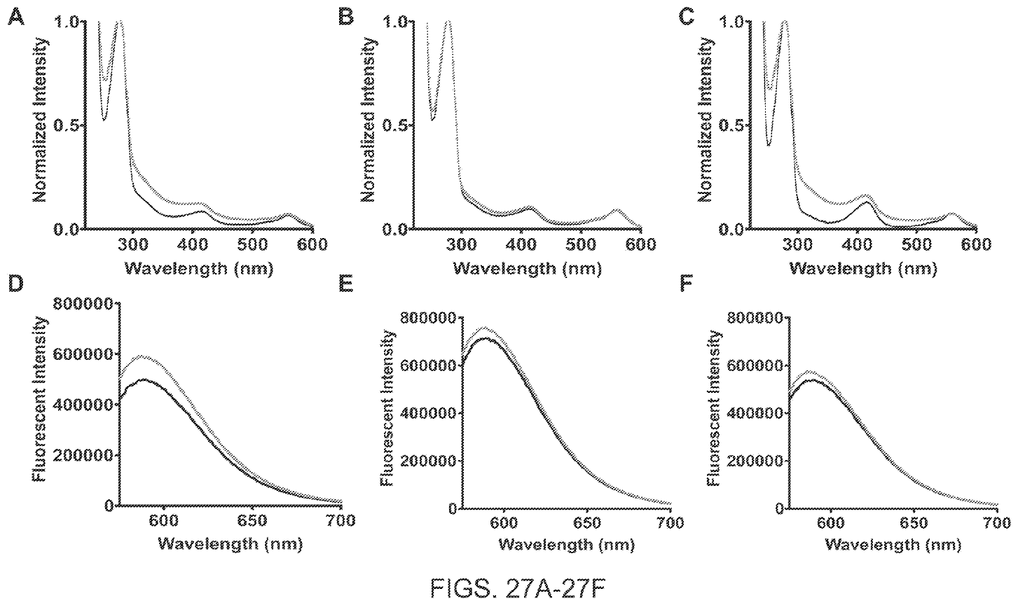

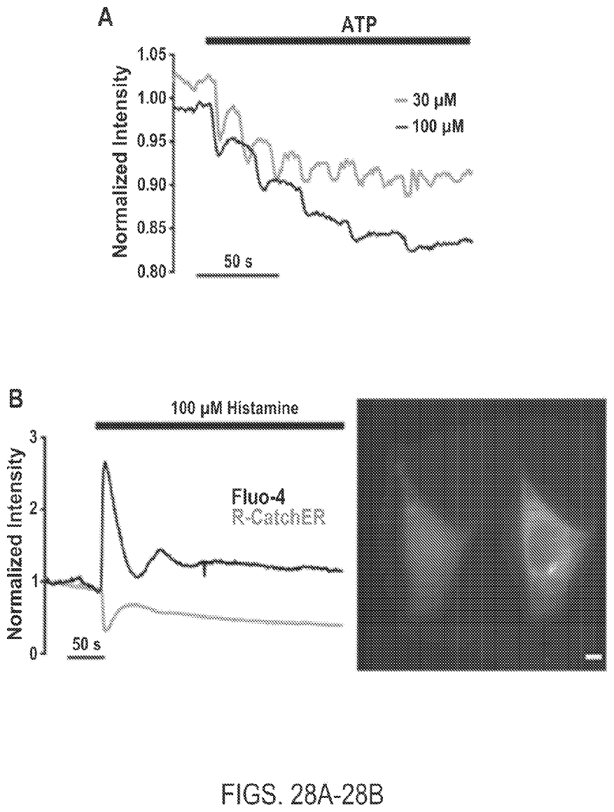

As for mRuby, a similar Ca²⁺ binding pocket was chosen. However, after the initial few attempts, we stopped moving forward on mRuby because of the low Ca²⁺ induced fluorescence change and low absorbance of the deprotonated state of the chromophore of mRubyP142ER198DH216EV218E, mRubyT144ER198DH216EV218E, and mRubyT144ER198DH216DV218E (FIG. 27).

Novel principle to design Ca²⁺ indicators with large Ca²⁺-induced fluorescence changes by tuning rapid (ns-μs) protein dynamic motions. Two series of red Ca²⁺ indicators were generated based on the scaffold fluorescent protein mApple and mCherry, respectively, by altering the electrostatic potential around the chromophore as done in developing the green Ca²⁺ indicator G-CatchER (Table 1). A putative single Ca²⁺-binding site was located on the surface of mApple (A145/E147/D196/K198/R216/E218). Among a series of 10 different mApple variants tested, in vitro absorption spectra of R-CatchER (mApple A145E/K198D/R216D) exhibited an increase in the protonated state relative to the deprotonated state (FIG. 2A). Ca²⁺-binding reversed the state of the chromophore in R-CatchER in vitro, with an increase of the deprotonated state relative to the protonated state in the absorption spectra and large Ca²⁺ induced fluorescence change (FIGS. 2A-2B). This alteration of the chromophore state is also supported by the observation of a large pKa decrease in R-CatchER upon Ca²⁺-binding (Table 2). The addition of a Ca²⁺-binding site and binding to Ca²⁺ also resulted in drastic changes in its biophysical properties (Table 2). However, this study failed in creating a mCherry-based red Ca²⁺ indicator with required dynamic range for Ca²⁺ induced fluorescence change despite the extensive efforts with >50 mutations. The best variant, MCD1 (mCherry A145E/S147E/N196D/K198D/R216E), only had a small fluorescence change upon Ca²⁺-binding (Table 3) and the smallest pKa change (4.31±0.01 at 0 mM Ca²⁺ versus 4.30±0.01 at 10 mM Ca²⁺), indicating that the original proposed design principle for Ca²⁺ indicators by altering local electrostatics was insufficient.

The success of R-CatchER (and previously, G-CatchER) and the negative result of MCD1 led to the next experiment to search for additional key principles for the design of Ca²⁺ indicators, besides localized electrostatics. One hypothesis is that designing a Ca²⁺ indicator with a large dynamic range requires a malleable fluorescent protein whose conformational ensemble can be tuned by mutations and Ca²⁺-binding. A rapid Ca²⁺ indicator can be achieved by taking advantage of the inherent flexibility of the protein to occupy multiple states, including the dominant functional (fluorescent) state, and optimizing the sequence of the single Ca²⁺-binding site to achieve automatic tuning of rapid (ns-μs) dynamics in response to Ca²⁺-binding. This hypothesis has been verified by molecular dynamics (MD) simulations for R-CatchER, G-CatchER, and MCD1.

Ca²⁺-binding in R-CatchER and G-CatchER reversed the effects of engineering the Ca²⁺-binding site, which is consistent with the in vitro experiments.

To further validate the design principle, the chromophore dynamics of all the 10 different mApple variants were compared (Table 4). Specifically, the ratio of conformational probability density with the chromophore RMSD around 0.3 Å (corresponding to the major peak of chromophore RMSD distribution in wildtype mApple and presumably representing the deprotonated state of the chromophore) between Ca²⁺-free and Ca²⁺-bound forms of each variant was computed (denoted by X₁), which was used to predict the extent of recovery of the wildtype-like optical property by Ca²⁺ binding. A strong positive correlation was observed between chromophore dynamical changes derived from MD and Ca²⁺-induced absorbance change from purified proteins with respect to the deprotonated state of the chromophore (FIG. 2 and Table 5). This result supports, as a proof of concept, this design principle of GECIs by showing that adjustable (Ca²⁺ dependent) optical properties can be achieved by tuning intrinsic dynamics of an appropriate fluorescent protein via mutating the Ca²⁺ binding site and demonstrates MD as a powerful tool to quantitatively map sequence to function.

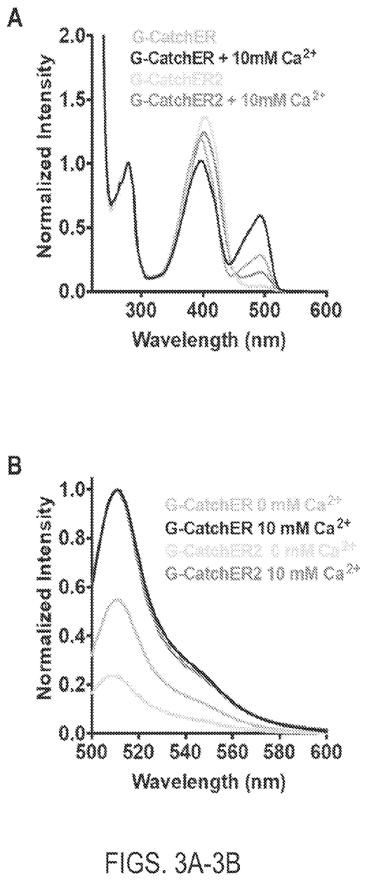

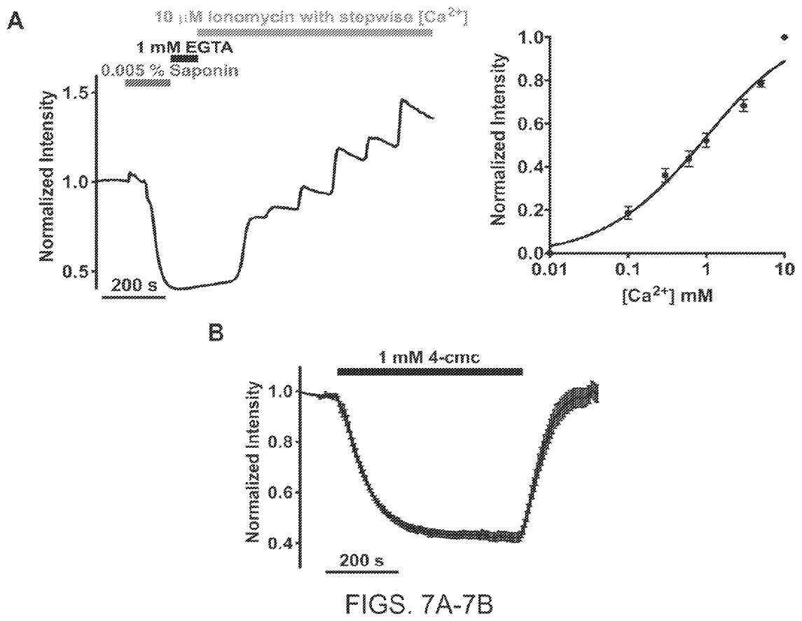

G-CatchER2 exhibited a significantly improved Ca²⁺ induced fluorescence change (3.9-fold compared to 1.9-fold) due to a near almost total conversion of the deprotonated state to the protonated state via stronger electrostatic repulsion (FIGS. 3A-3B). To ensure the capacity of G-CatchER2 to accurately monitor ER Ca²⁺ levels, the Ca²⁺ binding affinity (1.39±0.22 mM, N=14) in HeLa cells was determined with stepwise Ca²⁺ concentrations (FIG. 7A) which is similar to that determined in vitro (Table 2). Upon application of a ryanodine receptor agonist, 4-cmc, a drastic fluorescence decrease was observed reflecting Ca²⁺ release from ER in C2C12 cells (ΔF/F=0.57±0.02, N=10), using highly inclined and laminated optical sheet (HILO) microscopy (FIG. 7B). Thus, the data clearly support the proposed principle in designing Ca²⁺ indicators.

In vitro ultrafast kinetics and characterization. R-CatchER was able to bind Ca²⁺ with a 1:1 stoichiometry and exhibited similar Ca²⁺-binding affinities close to ER Ca²⁺ concentrations (FIGS. 8A-8B, and Table 2). Concurrently, after binding Ca²⁺, the fluorescence quantum yield and brightness of R-CatchER increased, indicating that Ca²⁺-binding is coupled with a concomitant recovery of fluorescence (Table 2). Moreover, Ca²⁺ induced fluorescence changes of R-CatchER were insensitive to the addition of Mg²⁺, K⁺, and Na⁺, indicating their strong metal selectivity for Ca²⁺ (FIG. 7C).

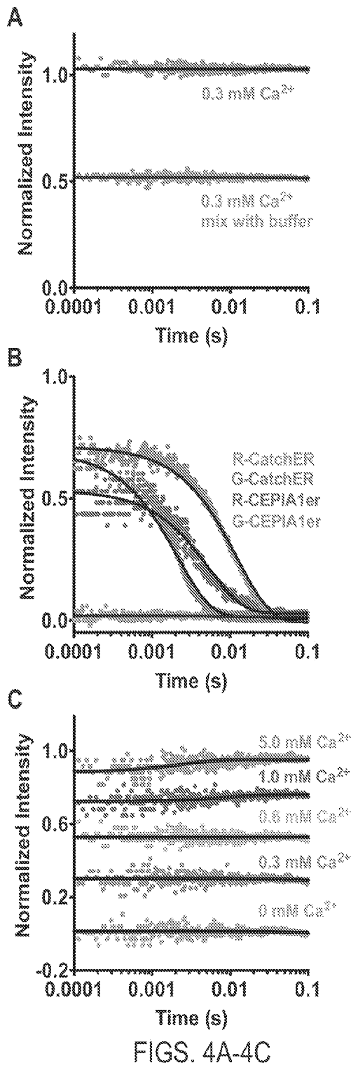

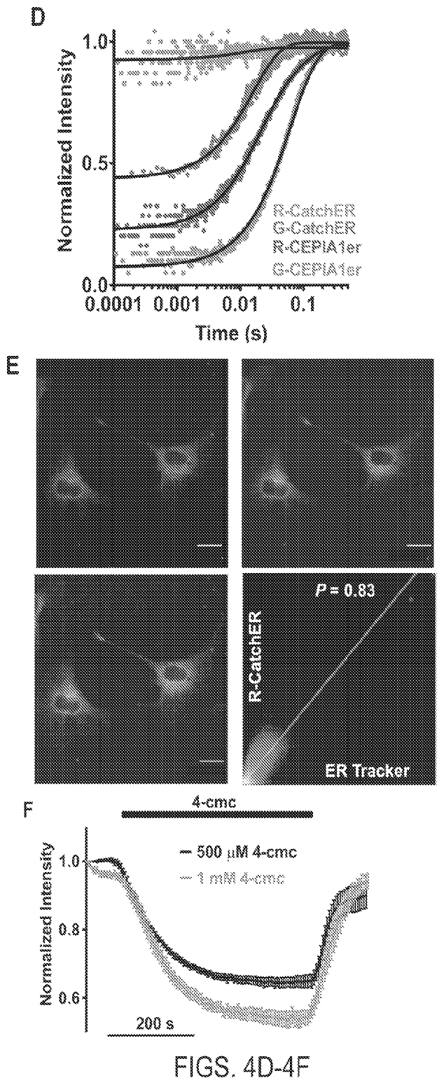

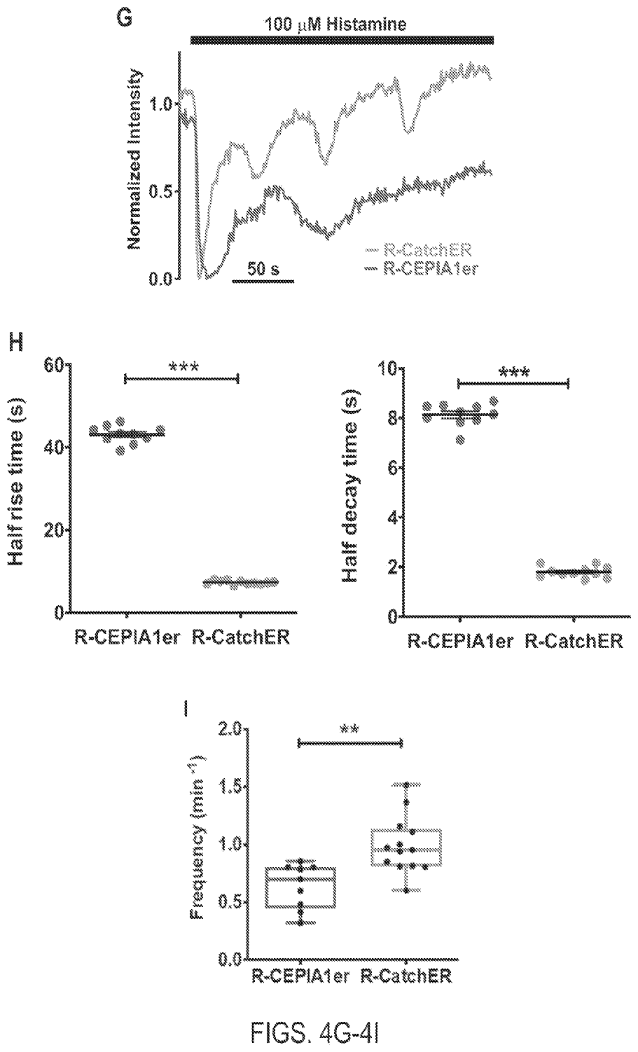

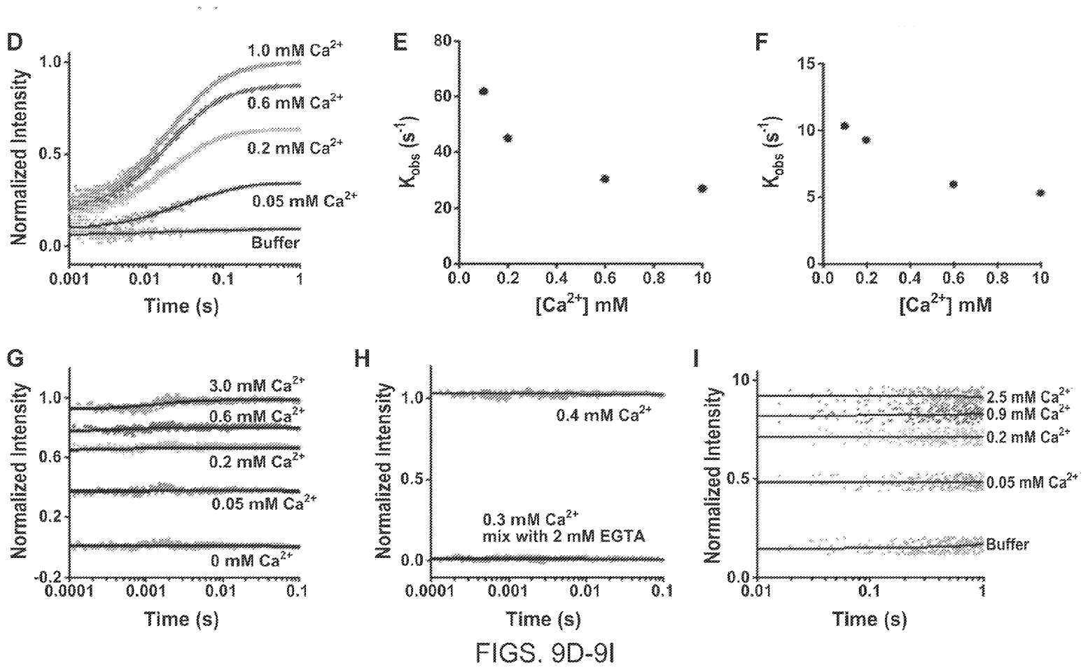

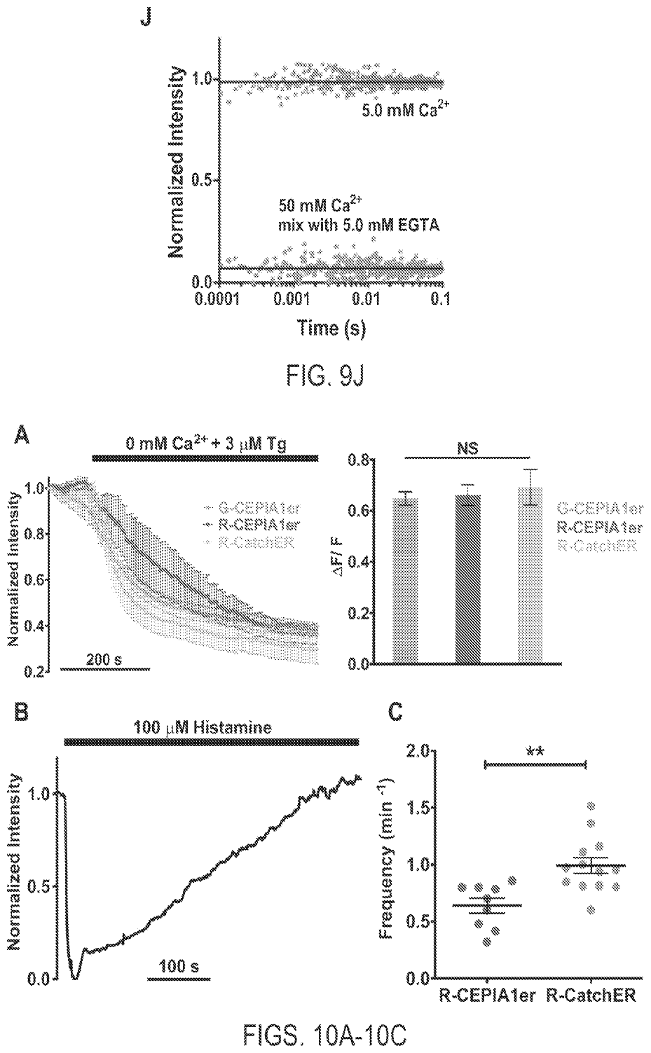

The Ca²⁺-binding kinetics of R-CatchER, and MCD1, was determined using stopped-flow spectrofluorometry. The decrease in fluorescence of R-CatchER occurred within the instrument's dead time (2.2 ms), indicative of ultrafast Ca²⁺ dissociation kinetics (k_{off}≥2×10³ s⁻¹) (FIG. 4A). This value was estimated based on six times ti/2 of the off rate being less than the dead time of the instrument, which was faster than G-CatchER (˜700 s⁻¹). Conversely, R-CEPIA1er and G-CEPIA1er showed much slower dissociation rates (183±5 s⁻¹ and 81±1 s⁻¹, respectively, p<0.0001) (FIG. 4B and FIGS. 9A-9B). R-CatchER also exhibited a rapid Ca²⁺ association rate, with an estimated kₒₙ≥7×10⁶ M⁻¹s⁻¹ (FIG. 4C). R-CEPIA1er and G-CEPIA1er had much slower Ca²⁺ association rates, due to multiple Ca²⁺-binding processes. The rate of Ca²⁺ association for R-CEPIA1er was 3.2×10⁵ M⁻¹s⁻¹, while the rate of Ca²⁺ association to G-CEPIA1er was 1.2×10⁵ M⁻¹ s⁻¹ (p<0.0001) (FIG. 4D and FIGS. 9C-9F). Both the increase and decrease of fluorescence of MCD1 were within the instrument's dead time (2.2 ms), with estimated kinetics of k_{off}≥2×10³ s⁻¹, and kₒₙ≥3×10⁷ M⁻¹s⁻¹ (FIGS. 9I-9J). The determined kinetic responses of R-CatchER, G-CatchER, and MCD1 indicated superior kinetics of the designed Ca²⁺ indicators over CaM based GECIs. Detailed biophysical properties comparisons with other GECIs are listed in Table 2.

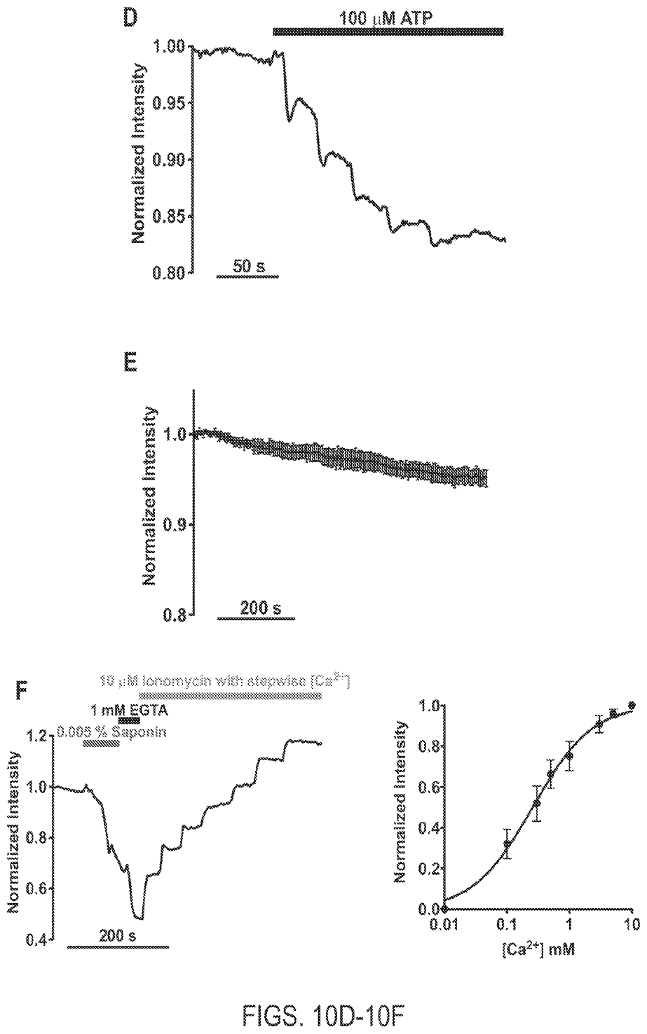

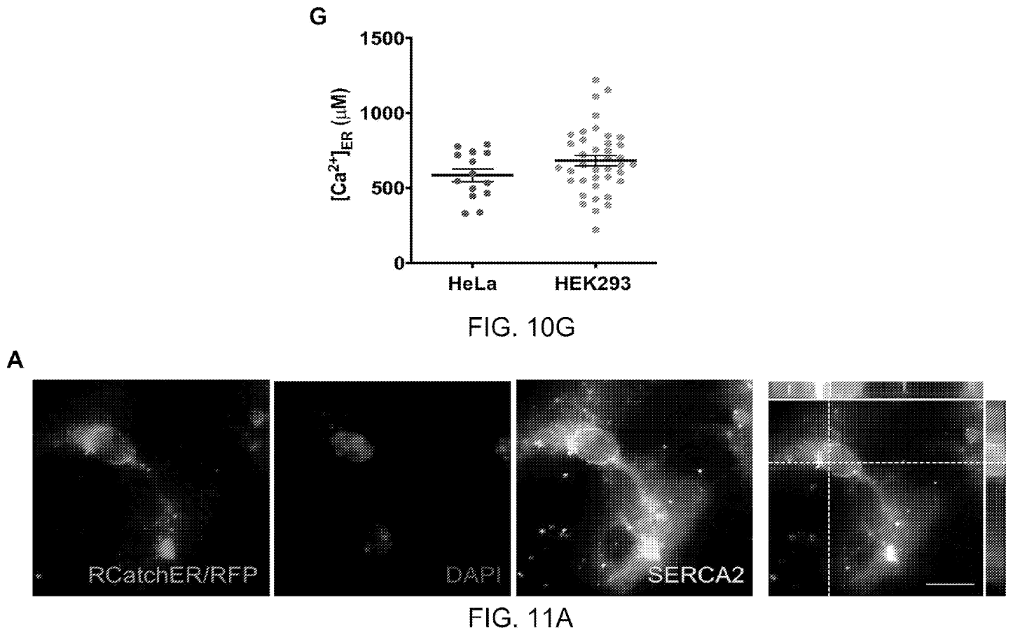

Detection of rapid spatiotemporal ER Ca²⁺ dynamics in multiple cell types. R-CatchER can be targeted to the ER by fusion of an ER targeting sequence calreticulin and an ER retention sequence KDEL (SEQ ID NO: 15), as verified by co-immunostaining of R-CatchER with ER-tracker green (FIG. 4E). Upon application of a ryanodine receptor agonist, 4-cmc, a dose-dependent fluorescence decrease was observed reflecting Ca²⁺ release from ER in C2C12 cells (0.5 mM, ΔF/F=0.36±0.01, N=9; 1.0 mM, ΔF/F=0.47±0.02, N=15), using HILO microscopy (FIG. 4F). R-CatchER, G-CEPIA1er, and R-CEPIA1er, all exhibited similar fluorescence decreases (ΔF/F=0.69±0.07, N=9; ΔF/F=0.64±0.03, N=10; ΔF/F=0.66±0.04, N=10, respectively) upon application of 3 μM Thapsigargin (Tg) (FIG. 10A). R-CatchER captured histamine (100 μM) induced ER Ca²⁺ oscillations in HeLa cells, with a half rise time of 7.3±0.1 s and a half decay time of 1.8±0.1 s of the first peak (FIGS. 4G-4H). In contrast, G-CEPIA1er was incapable of reporting ER Ca²⁺ oscillations and only exhibited a slow recovery without distinct oscillation peaks (FIG. 10B). With R-CEPIA1er, significantly slower rates with a half rise time of 43.1±0.7 s and a half decay time of 8.1±0.2 s of the first peak were observed (p<0.0001) (FIGS. 4G-4H and FIG. 10C). Importantly, R-CatchER also has the capability to report pathway dependent ER Ca²⁺ oscillations, with faster ER Ca²⁺ oscillations in HEK293 cells triggered by 100 μM ATP (2.57±0.60 min⁻¹, N=13) compared to those in HeLa cells triggered by 100 μM histamine (1.00±0.25 min⁻¹, N=8) (FIG. 10D).

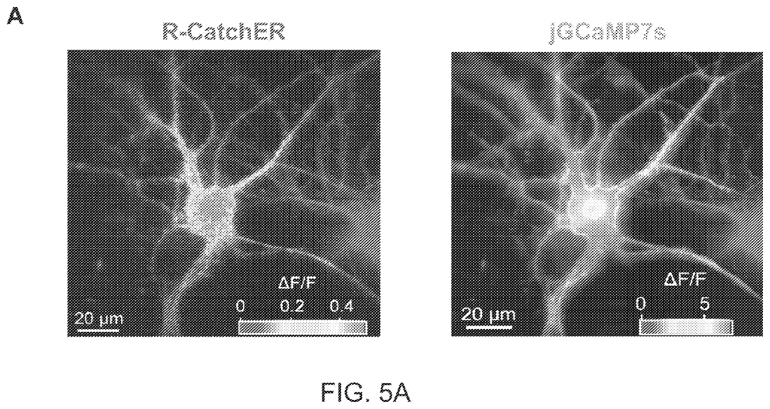

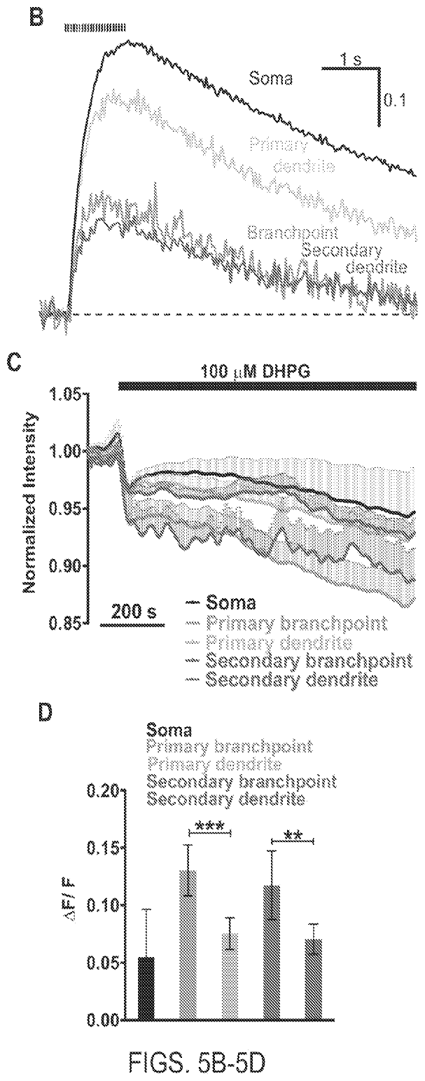

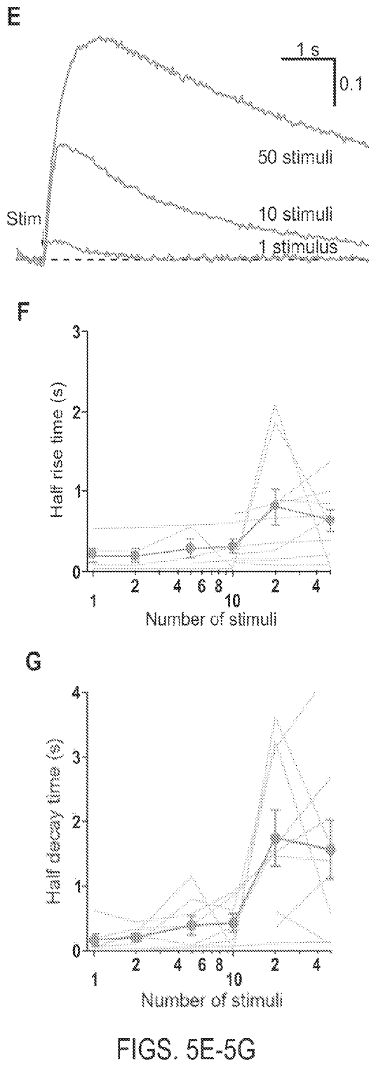

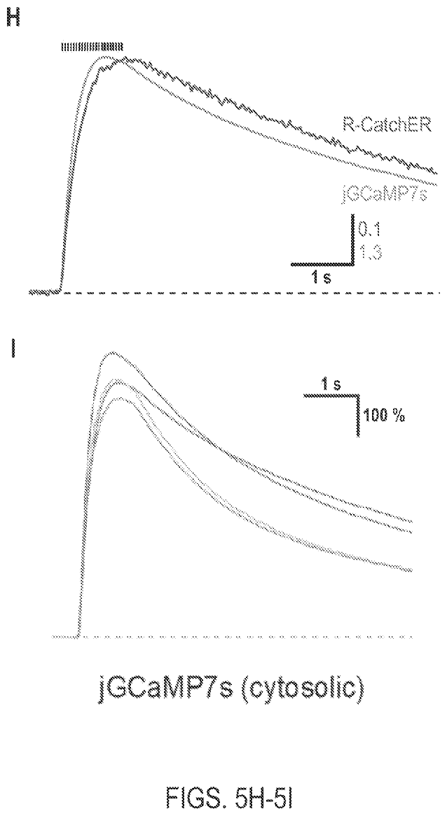

The next experiment examined the capability of R-CatchER to report rapid overloading or release of Ca²⁺ in the ER of isolated primary neurons in culture. Upon field electrical stimulation of 50 Hz for 1 s, widespread transient Ca²⁺ increases were observed in the ER, with significantly varying levels depending on the cell compartments (ΔF/F; soma: 0.173±0.048; primary dendrites: 0.083±0.039; branchpoints: 0.077±0.022; secondary dendrites: 0.036±0.017; *p=0.004, N=9 cells; FIGS. 5A-5B).

The role of the ER as a source of Ca²⁺ was examined using a different stimulus. Upon application of the group I mGluR agonist, (S)-3,5-dihydroxyphenylglycine (DHPG; 100 μM), decreases of R-CatchER fluorescence were observed with significantly differing levels between dendrites and primary branchpoints (primary, 0.13±0.02 and 0.08±0.01, p<0.0001, N=4, secondary, 0.12±0.03 and 0.07±0.01, p<0.001, N=4) (FIGS. 5C-5D). These findings are in agreement with the formation of Ca²⁺ waves, with hotspots initiating at dendritic branchpoints due to the clustering of IP₃ receptors in this region.

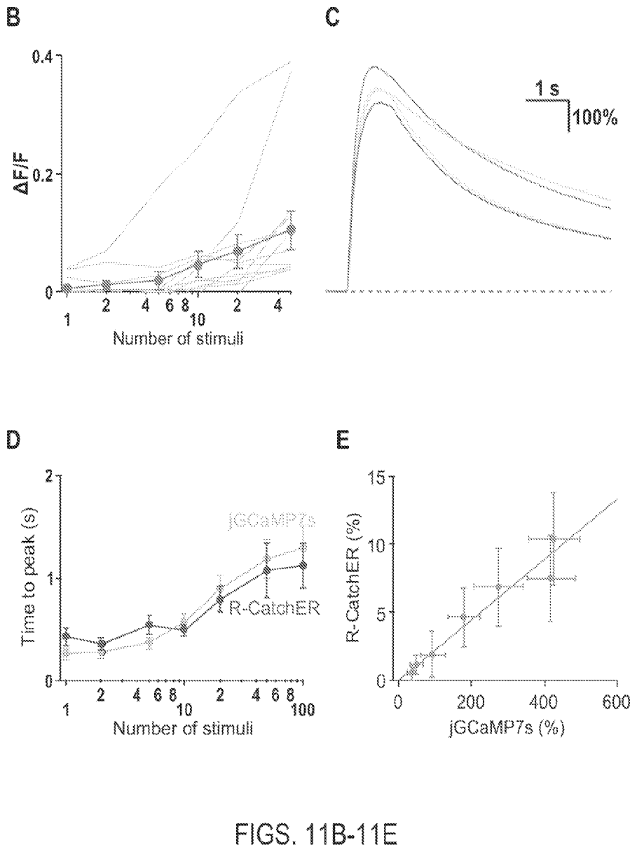

To evaluate the sensitivity and linearity of R-CatchER responses, the number of electrical stimuli was varied. In some cells, Ca²⁺ transients in ER were readily detectable even with a single stimulus (FIG. 5E), confirming superior kinetics and sensitivity of R-CatchER. Overall, R-CatchER fluorescence increased linearly with the number of stimuli in the range tested (up to 50 stimuli) (FIG. 11B). The half rise time and half decay time of R-CatchER indicated fast kinetics of ER Ca²⁺ loading (FIGS. 5F-5G). Next, a cytosolic Ca²⁺ indicator, jGCaMP7s, was co-expressed to illustrate the multi-color imaging capability of R-CatchER (FIG. 5H). Unlike ER Ca²⁺, amplitudes of cytosolic Ca²⁺ transients were not significantly different among different subcellular compartments (ΔF/F; soma: 5.50±0.60; primary dendrites: 4.60±0.61; branchpoints: 5.20±0.79; secondary dendrites: 3.14±0.58; p=0.07, N=9) (FIG. 5A and FIG. 11C). This nondistinctive spatial profile was consistent with Ca²⁺ influx mediated by back-propagation of action potential into dendrites, which is much faster than the CaM-based Ca²⁺ indicators. No significant difference in time to peak was observed between both R-CatchER and jGCaMP7s (FIG. 11D). Additionally, although R-CatchER most likely accurately reported the decay of ER Ca²⁺ with its superior off rate (2×10³ s⁻¹) (FIG. 4A), the decay of cytosolic Ca²⁺ reported with jGCaMP7s is exaggerated due to the slow kinetics of the Ca²⁺ indicator (2.87 s⁻¹). The amplitude of the ER Ca²⁺ transient measured with R-CatchER correlated with that of cytosolic Ca²⁺ transients of jGCaMP7s over the entire range tested (FIG. 11E).

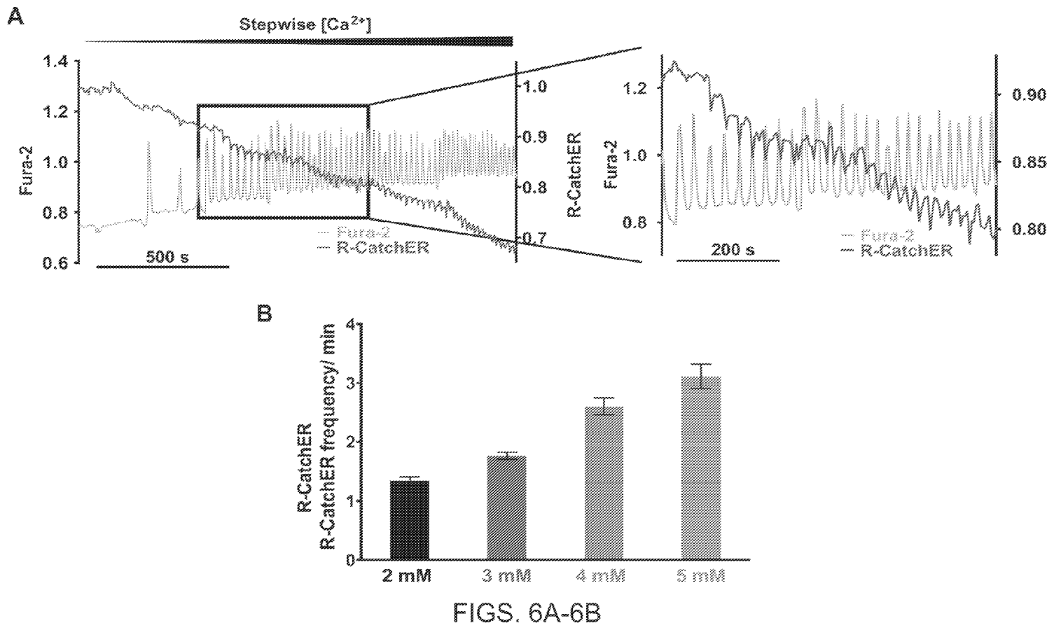

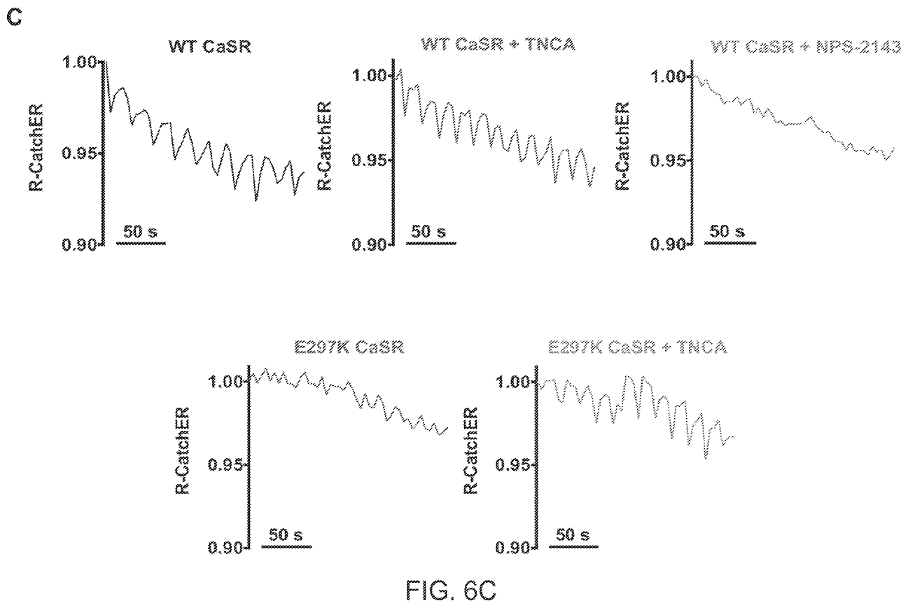

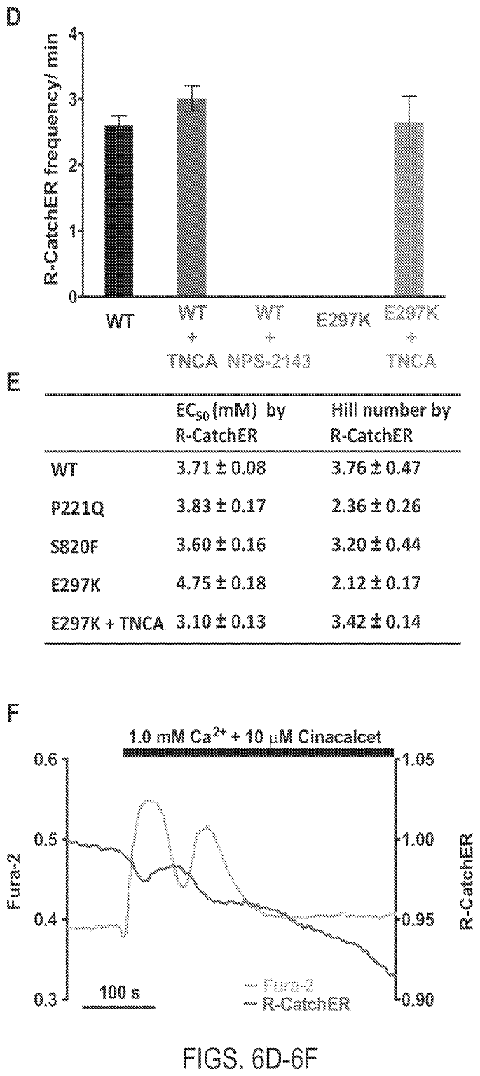

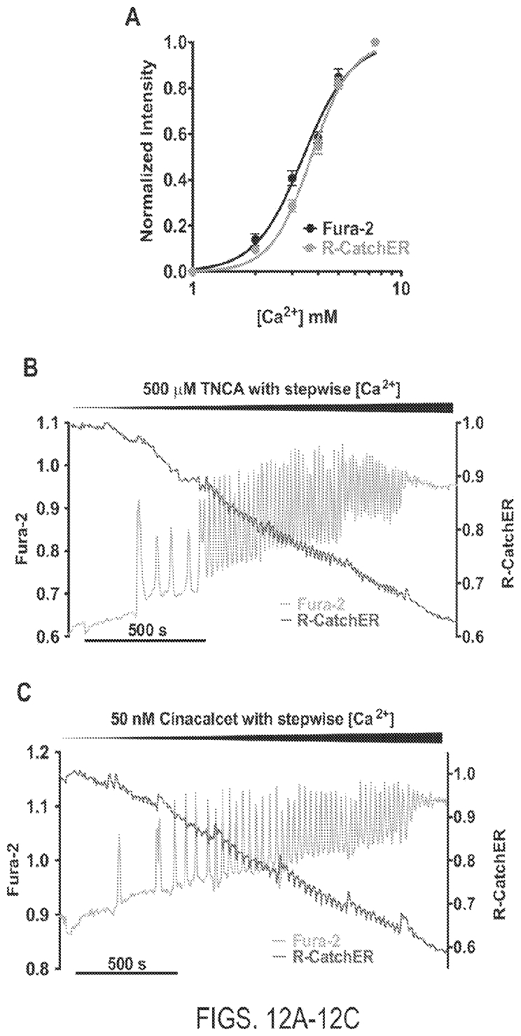

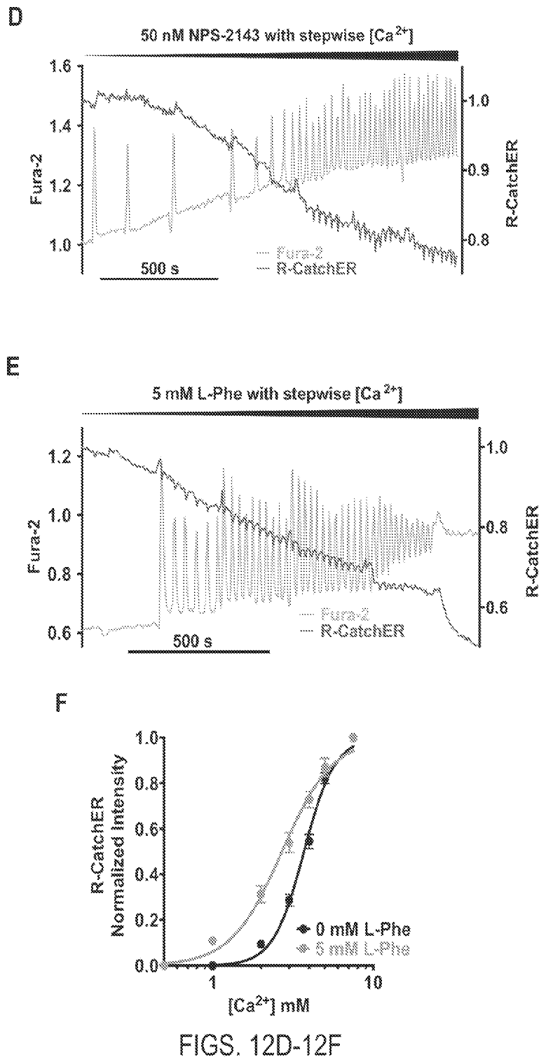

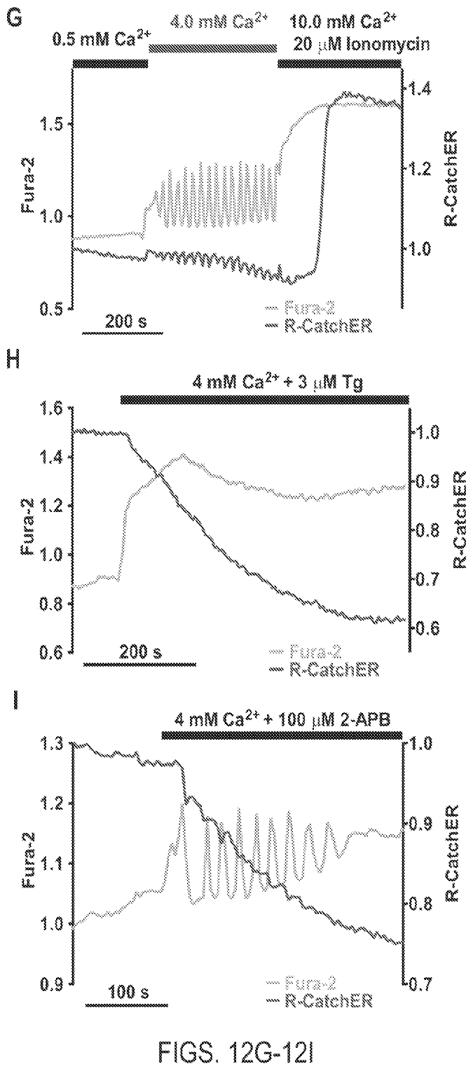

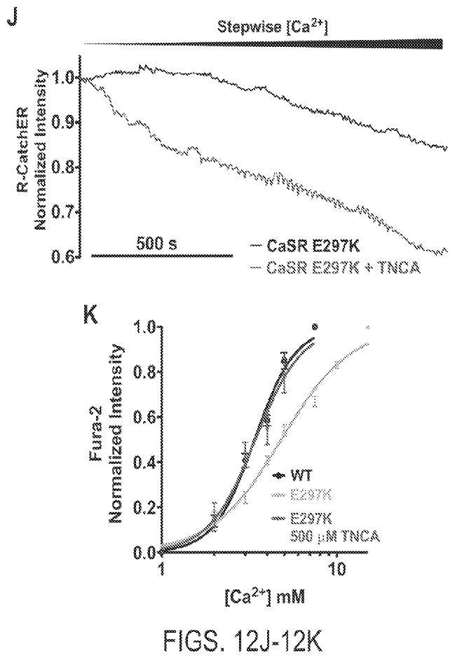

Direct observation of CaSR mediated ER Ca²⁺ oscillations via extracellular stimuli. How Ca²⁺-sensing receptor (CaSR) and other GPCRs are able to respond to extracellular Ca²⁺ and other stimuli to trigger ER-mediated cytosolic Ca²⁺ oscillations/mobilizations and their roles in diseases remain unclear. Here, the study reported the first direct observation of ER Ca²⁺ oscillation by Ca²⁺-sensing receptor (CaSR). GFP-tagged CaSR and R-CatchER were expressed in HEK293 cells and cytosolic and ER Ca²⁺ was monitored by Fura-2 and R-CatchER, respectively. Increasing the extracellular Ca²⁺ concentration increased the frequency of ER Ca²⁺ oscillations that mirrored the cytosolic Ca²⁺ oscillations (FIGS. 6A-6B). The EC₅₀ of CaSR activation was determined with R-CatchER to be 3.71±0.08 mM (N=43), consistent with reported EC₅₀ by cytosolic Ca²⁺ responses (FIG. 12A). Both cytosolic and ER Ca²⁺ oscillations were eliminated by various pharmacological interventions including a SERCA blocker, Tg (3 μM), an IP₃R blocker, 2-Aminoethoxydiphenyl borate (2-APB; 100 μM), and saturation of ER Ca²⁺ by a Ca²⁺ ionophore, ionomycin (20 μM), in the presence of 10 mM Ca²⁺ (FIGS. 12G-12I). L-Phe, L-1,2,3,4-tetrahydronorharman-3-carboxylic acid (TNCA), and a positive allosteric modulator, Cinacalcet, in the presence of Ca²⁺ increased the frequency of ER Ca²⁺ oscillations, while the addition of a CaSR negative allosteric modulator, NPS2143, decreased the frequency (FIG. 6C and FIGS. 12B-12E). In addition, using R-CatchER, L-Phe cooperatively potentiated ER Ca²⁺ mobilization by extracellular Ca²⁺, resulting in an EC₅₀ of 2.70±0.10 mM (N=21) (FIG. 12F).

Many mutations in CaSR have been shown to be associated with homotropic/heterotropic cooperativity and lead to calcitropic and non-calcitropic diseases. To unveil a molecular mechanism of ER Ca²⁺ in these diseases, HEK293 cells were co-transfected with R-CatchER and one of the disease-associated mutations of CaSR (P221Q, E297K, and S820F). P221Q and E297K mutants significantly decreased the sensitivity and cooperativity of CaSR to changes in extracellular Ca²⁺, with an EC₅₀ of 3.83±0.17 mM (N=24, p=0.0002) and 4.75±0.18 mM (N=26, p<0.0001), respectively, compared to the wildtype (3.71±0.08 mM, N=31) using R-CatchER (FIG. 6E). Conversely, with the active mutation of S820F, a significant increase of the sensitivity was observed (3.60±0.16 mM, N=29, p<0.01) (FIG. 6E). Importantly, TNCA (500 μM) was able to reverse the effect of E297K mutation on both sensitivity (3.10±0.13 mM, N=20, p<0.0001) and cooperativity (Hill coefficient: 3.08±0.40 with TNCA vs. 2.12±0.18 without TNCA), supporting co-activation working model (FIGS. 6C-6E and FIGS. 12J-12K). In addition, R-CatchER was able to detect ER Ca²⁺ oscillations triggered by 1.0 mM extracellular Ca²⁺ and 10 M of the CaSR allosteric activator Cinacalcet in a human medullary C cell carcinoma cell line TT, with wider and lower frequent peaks (FIG. 6F).

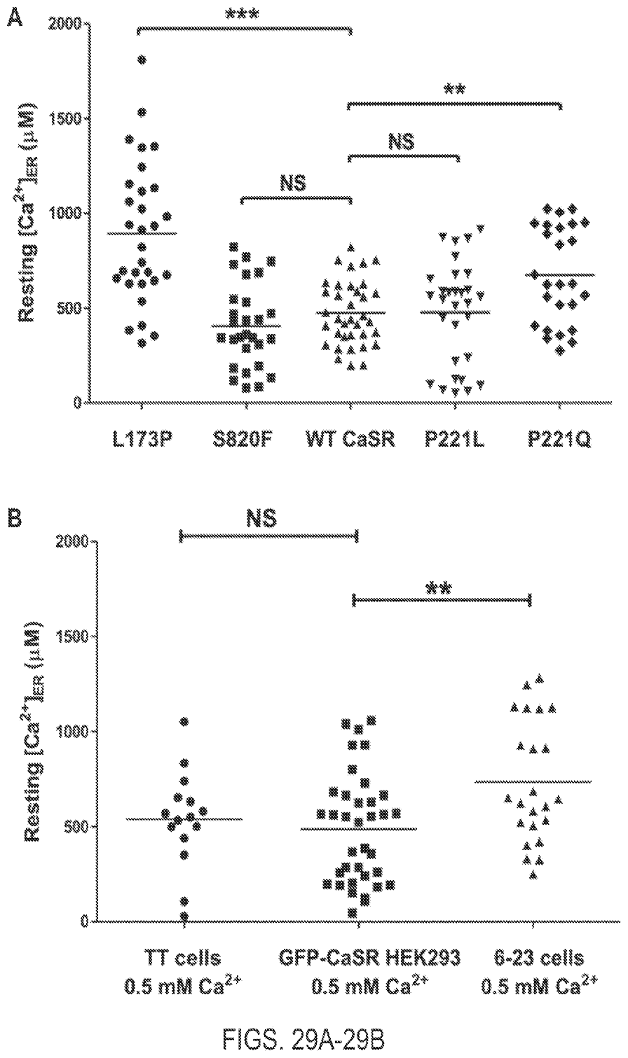

R-CatchER was then used to quantitative measure Basal [Ca²⁺ ]ER in different CaSR mutations to uncover the crosstalk between extracellular Ca2+ and ER Ca²⁺. For the gain-of-function mutation S820F (406.1±41.4 μM) and P221L (477.7±49.4 μM), there is no significant difference in comparison to wildtype CaSR under 1.8 mM Ca²⁺. However, there is a significant difference of the loss-of-function L173P (893.6±67.6 μM, p<0.0001) and P221Q (674.6±50.2 μM, p<0.01) compared to wildtype CaSR under 1.8 mM Ca²⁺. These data were confirmed by using different cell lines. No difference was observed between TT cells (539.0±65.9 μM) and GFP-CaSR (487.1±48.2 μM) under 0.5 mM Ca²⁺, while a significant difference was observed between 6-23 cells (735.0±66.7 μM) and GFP-CaSR (487.1±48.2 μM) under 0.5 mM Ca²⁺ (FIG. 29).

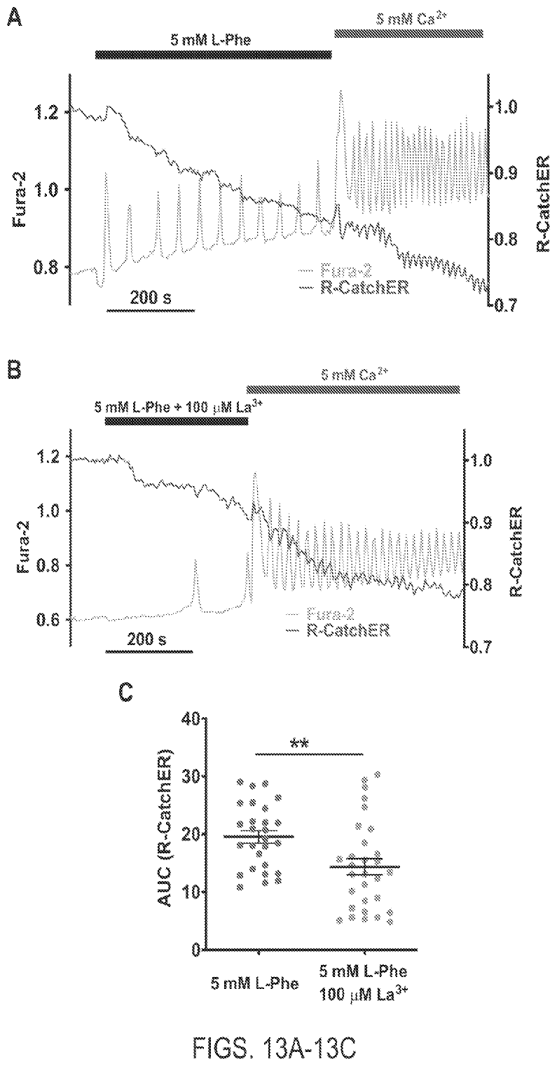

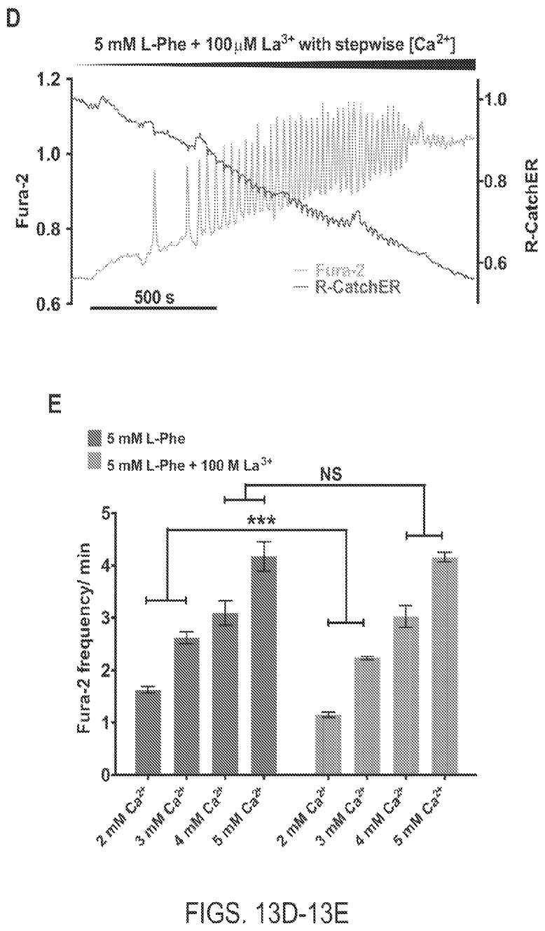

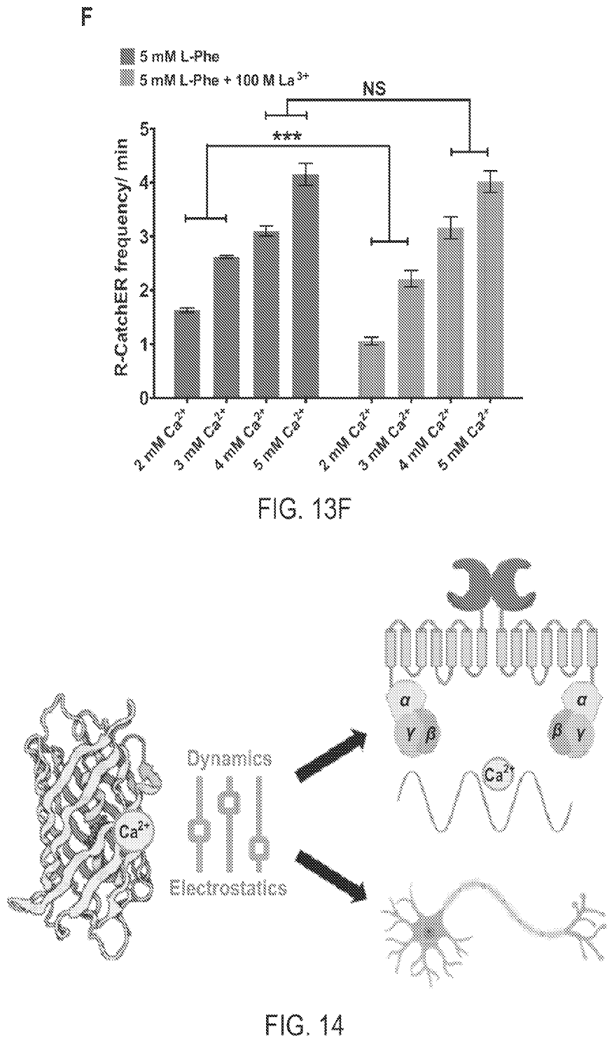

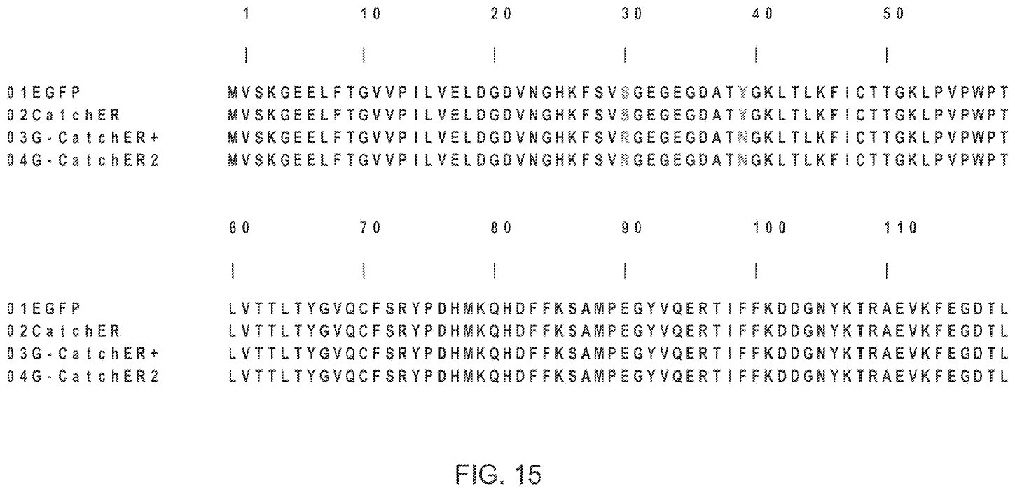

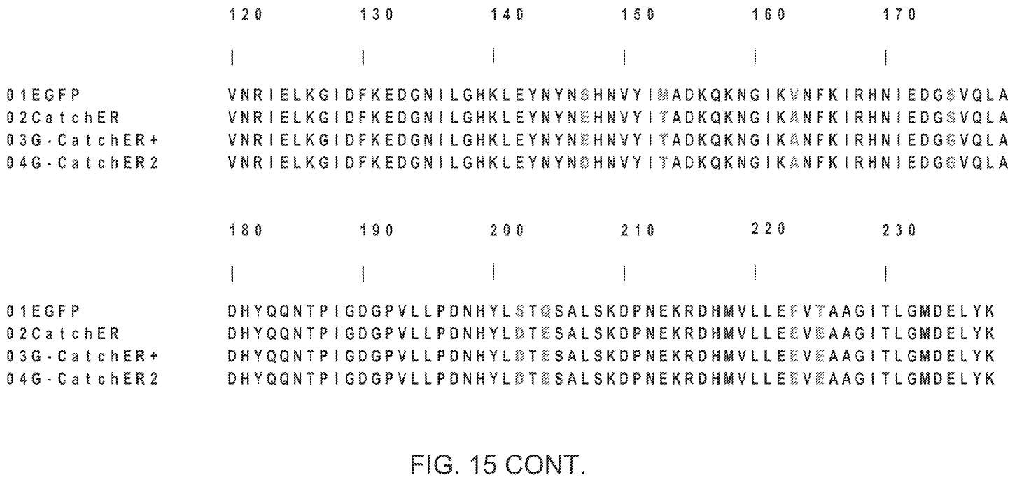

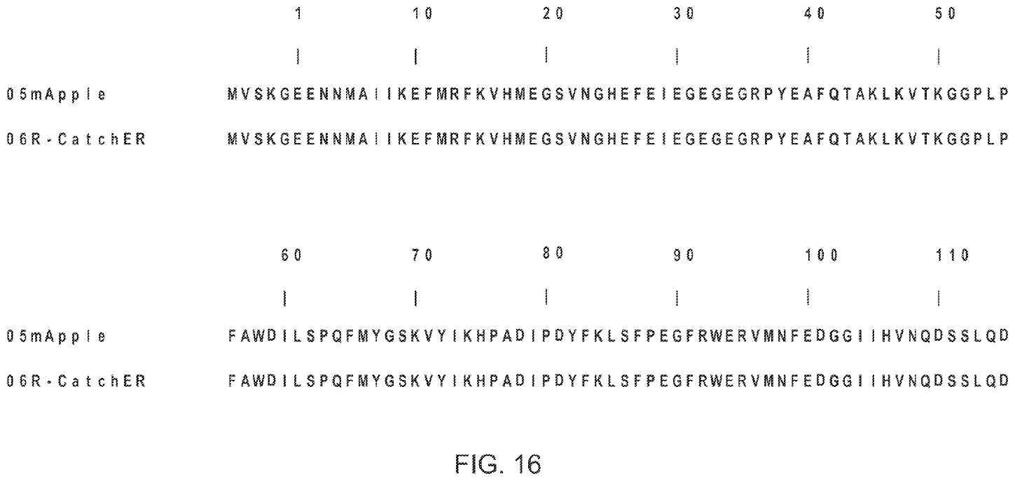

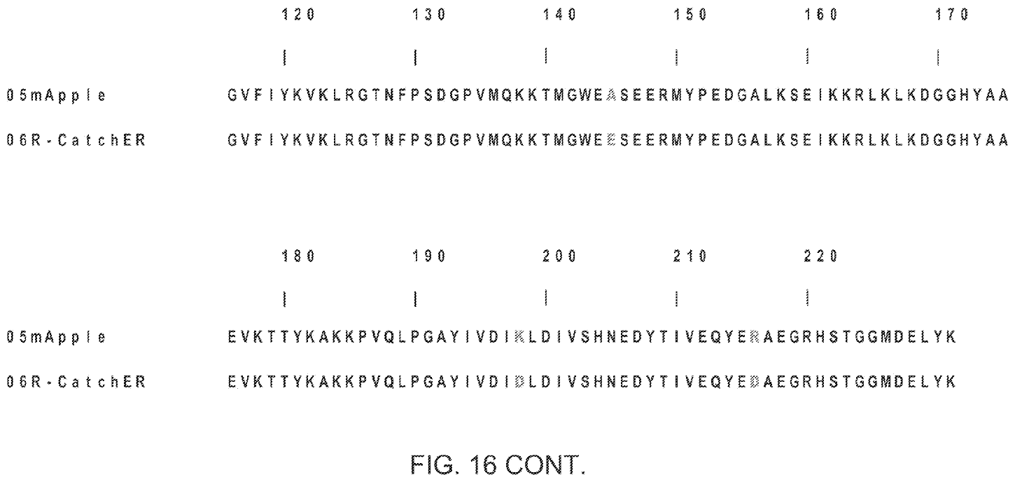

Estimated absolute ER Ca²⁺ concentration in different CaSR mutations using R-CatchER. B. Estimated absolute ER Ca²⁺ concentration in different cell lines using R-CatchER. The next experiment addressed the origin and contribution of intracellular Ca²⁺ oscillation mediated by CaSR using our developed R-CatchER. It was suggested that aromatic amino acids in the presence of Ca²⁺ activates CaSR and induces activation of the heterotrimeric GTP binding proteins G12/13, leading to RhoA activation and Ca²⁺ influx. However, such a mechanism has never been directly reported due to a lack of sensitive ER-based Ca²⁺ indicators. It remains unclear that how much ER Ca²⁺ release contributes to the cytosolic Ca²⁺ oscillation, compared to Ca²⁺ influx from the extracellular fluid. First, to unambiguously report the ER and cytosolic Ca²⁺ oscillation, we apply 3 μM Tg to block SERCA for ER refilling and 100 μM 2-Aminoethoxydiphenyl borate (2-APB) to block IP₃R, resulting in the elimination of both ER and cytosolic Ca²⁺ oscillation (FIG. 13). Additionally, 20 μM ionomycin in the presence of 10 mM Ca²⁺ abolished 4 mM extracellular Ca²⁺ trigger intracellular and ER Ca²⁺ oscillation (FIG. 13). Second, applying 5 mM L-Phe under 0.5 mM Ca²⁺ then following with 5 mM Ca²⁺ in HEK293 transfected with GFP-CaSR and R-CatchER, two different Ca²⁺ oscillation patterns were detected from the signals of Fura-2 and R-CatchER, which L-Phe induced transient one but sinusoidal from extracellular Ca²⁺ (FIG. 13). Applying 100 M La³*, which blocks the L-type Ca²⁺ channel, diminished the L-Phe induced Ca²⁺ transient oscillation. But a decreased signal of R-CatchER was observed, indicating blocking the Ca²⁺ channel also leads to a decrease of the Ca²⁺ release from the ER (FIG. 13). By analyzing the area under the curve (AUC) of these two sets of experiments, there is a significant decrease of 100 μM La³++5 mM L-Phe (14.35±1.41, n=27), compared to 5 mM L-Phe alone (19.54±1.08, n=30, P<0.01) (FIG. 13). Additionally, the stepwise concentration of extracellular Ca²⁺ in the solution containing constant 5 mM L-Phe, both frequencies of Fura-2 and R-CatchER increased, giving the fact that L-Phe is an agonist to CaSR. Whereas stepwise concentration of extracellular Ca²⁺ in the solution containing constant 5 mM L-Phe+100 μM La³⁺, a significantly decreased frequency at 2 mM Ca²⁺ and 3 mM Ca²⁺ (P<0.0001) was observed, but surprisingly no difference under 4 mM Ca²⁺ and 5 mM Ca²⁺ (FIG. 13).

These results show a new mechanism, although La³⁺ can block partly intracellular Ca²⁺ transient oscillation induced by L-Phe, most of such Ca²⁺ change is from ER. It is further concluded that under low extracellular Ca²⁺ (3 mM Ca²), intracellular Ca²⁺ oscillation induced by L-Phe with Ca²⁺ mainly originated from both extracellular fluid and ER, which can be partially blocked by La³. However, under higher extracellular Ca²⁺ (≥3 mM Ca²⁺), intracellular Ca²⁺ oscillation induced by L-Phe with Ca²⁺ only came from ER.

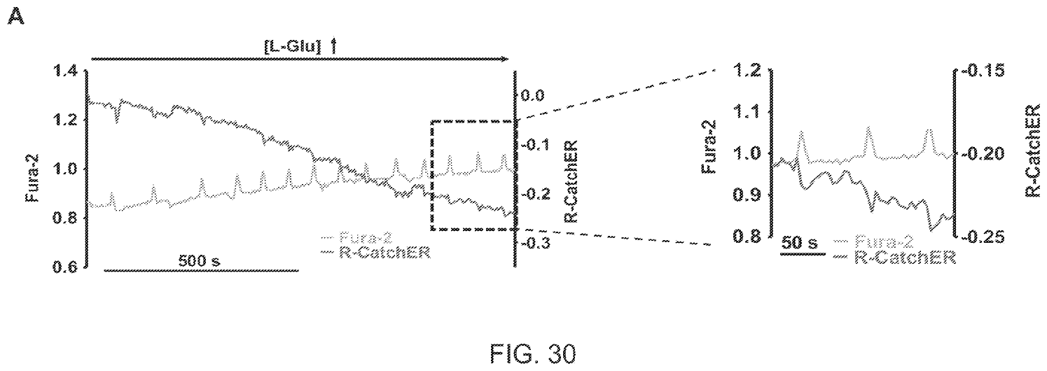

Intracellular Ca²⁺ oscillation/mobilization through G_{αq} signaling is also mediated by metabotropic glutamate receptors (mGluRs). However, again, the quantification of intracellular Ca²⁺ dynamics via IP₃R, which induced ER Ca²⁺ release, relays on indirect and convoluted intracellular Ca²⁺ responses by Ca²⁺ dyes. This study reported the direct measurement of ER Ca²⁺ releases and Ca²⁺ oscillation mediated by mGluR5 using R-CatchER. After co-transfecting R-CatchER with mGluR5 in HEK293 cells, simultaneously measurement of the intracellular Ca²⁺ using Fura-2 and ER Ca²⁺ was performed using R-CatchER. Increasing concentration of neurotransmitter L-glutamate (L-Glu) results in synchronized ER and cytosolic Ca²⁺ transient peaks (FIG. 30).

### Example 3. Discussion

The unprecedented rapid on and off rates of R-CatchER measured in vitro also enable the observation of stimuli-dependent differential ER Ca²⁺ dynamics mediated by various receptors, channels, and pumps. Although G-CEPIA1er and R-CEPIA1er were able to detect ER Ca²⁺ oscillations under 10 μM histamine or 30 μM ATP, the slow recovery phase with low oscillation frequency is due to a combination of slow kinetics and interference of modulation by CaM, which interacts with IP₃Rs and SERCAs (FIG. 4H and FIGS. 10B-10C). Additionally, R-CatchER was able to detect spatiotemporal profiles of ER Ca²⁺ release and refilling, by fast and slow stimuli in neurons. R-CatchER exhibited sensitivity in detection of a single stimulus in neurons. Additionally, these DHPG results show branchpoints contain a high reservoir of ER Ca²⁺ stores or a cluster of IP₃Rs, which is in contrast to a previous study using intracellular Ca²⁺ dye fluo-4.

Moreover, using R-CatchER, this study reports the first direct observation of ER Ca²⁺ oscillation, which directly links the extracellular, cytosolic, and ER compartments mediated by CaSR (FIG. 6, FIG. 12, and FIG. 13) to the detector developed herein detects that extracellular Ca²⁺ and agonists, such as L-Phe and TNCA, cooperatively tune ER Ca²⁺ oscillations mediated by CaSR. Importantly, it was shown for the first time that disease mutations largely alter ER Ca²⁺ responses, oscillation frequency and cooperativity. These results largely support the co-activation working model based on the structural determination. R-CatchER is invaluable as a tool to elucidate the molecular mechanisms mediated by CaSR and other GPCRs that integrates Ca²⁺ signaling. R-CatchER greatly expands the capability to visualize Ca²⁺ dynamics and can be applied to drug discovery for diseases related to ER dysfunction and Ca²⁺ mishandling.

### Example 4. Methods and Materials

Chemicals and Reagents. The E. coli. strain DH5a and the plasmid vector pCDNA3.1(+) were purchased from Invitrogen. Restriction enzymes, T4 DNA ligase, and T4 polynucleotide kinase (PNK) were purchased from New England Biolabs. Pfu DNA polymerase was purchased from G-Biosciences. The plasmid pRSETb was used. DNA sequencing for all clones was carried out by GENEWIZ Inc. The plasmid extraction was carried out using the QIAGEN mini-prep and maxi-prep kits. The Rosetta gami DE3 was obtained from Novagen for protein expression. The FPLC system (AKTA prime and AKTA FPLC), and the Ni-chelating Hi-Trap column were purchased from GE Healthcare. C2C12, HEK293, and HeLa cells were purchased from American Type Culture Collection (ATCC). (S)-3,5-DHPG and thapsigargin were obtained from Tocris. 4-cmc, histamine, and ATP were purchased from Sigma-Aldrich. ER-Tracker Green and ProLong gold antifade mountant with DAPI were obtained from Invitrogen. pCMV-G-CEPIA1er and pCMV-R-CEPIA1er were used. The jGCaMP7s gene in an adeno-associated virus 2 transfer vector with the human synapsin 1 promoter was purchased (Addgene 104487, Douglas Kim).

Cloning, protein expression and purification. mApple, EGFP, and mCherry variants were created by site-specific mutagenesis from parental scaffold mApple EGFP, and mCherry using Pfu DNA polymerase. All the DNAs for in vitro protein expression were subcloned into pRSETb with the BamH1 and EcoR1 restriction sites. To target the proteins in the endoplasmic reticulum (ER) lumen for cell imaging, the DNAs were subcloned into pCDNA3.1(+) vector by the same enzymes BamHI and EcoRI. ER retention sequence KDEL (SEQ ID NO: 15) was fused to the C-terminal before the stop codon and ER targeting sequence of calreticulin MLLSVPLLLGLLGLAAAD (SEQ ID NO: 16) was inserted to the N-terminal. Proteins were expressed by Rosetta gami(DE3). After IPTG induction and after OD reached 0.6, the temperature was lowered to 25° C. The protein was purified using the Ni²⁺ chelating column. R-CEPIA1er and G-CEPIA1er were subcloned from pCMV into pRSETb. The same expression procedures were used for R-CEPIA1er and G-CEPIA1er in BL21 (DE3) cells.

Calcium (Ca²⁺) binding assay. 10 μM protein samples of mApple, EGFP, and mCherry variants were titrated with different concentrations of Ca²⁺. Data were fitted with a 1:1 binding equation. Fluorescence intensities were collected using a Spectrofluorimeter (Photon Technology International, Inc.) and the absorbance values of Ca²⁺-free and Ca²⁺-loaded forms were determined using a Shimadzu UV-1601 spectrophotome.

pKa determination. To measure the chromophore pKa of mApple, EGFP, and mCherry variants, the proteins were prepared in buffers (sodium acetate buffer for pH 3-5, MES buffer for pH 5-6, HEPES buffer for pH 6.5-8, TRIS buffer for pH 8.5-9) covering a pH range from 3 to 10. All samples were Incubated at 4° C. overnight, then the following day, the absorbance and fluorescence spectra were collected using a Shimadzu UV-1601 spectrophotometer and the Spectrofluorimeter.

Quantum yield, extinction coefficient, and brightness determination. The quantum yield values of all the variants were determined by measuring the emitted fluorescence intensities and absorbance intensities of the chromophore at different protein concentrations. The wildtype was used as a reference to calculate quantum yield. Brightness was defined as a visual perception in which a source appears to emit or reflect a given amount of light, which was obtained by multiplying the extinction coefficient and the quantum yield.

In vitro kinetics by stopped flow spectrofluorometer. The kinetics were determined by a Hi-Tech SF-61 stopped-flow spectrofluorometer equipped with the mercury-Xe lamp (10 mm path length, dead time of 2.2 ms) at 20° C. For R-CatchER and its variant or R-CEPIA1er, excitation was at 569 nm and a long-pass 590 nm filter was used. For G-CEPIA1er, a 530 nm long-pass filter was set with excitation at 498 nm, while for MCD1, excitation at 587 nm and a long-pass 600 nm filter were applied. For association kinetics, R-CatchER, R-CatchER variant, MCD1, R-CEPIA1er, and G-CEPIA1er were mixed with the same buffer containing an increasing concentration of Ca²⁺. For disassociation kinetics, R-CatchER, R-CatchER variant, MCD1, R-CEPIA1er, and G-CEPIA1er in buffers with concentration of Ca²⁺ at K_{d}, were mixed with 5 mM EGTA or buffer. The raw data were fitted using either single exponential for R-CatchER, R-CatchER variant, and MCD1, or double exponential equations for R-CEPIA1er and G-CEPIA1er.

Electrostatic potential calculation. Electrostatic potentials were calculated using Adaptive Poisson-Boltzmann Solver (APBS) 1.4 through the APBS plugin v1.3 of VMD. The dielectric constant for the protein interior was set to 2.0. Default values were used for other parameters (i.e., briefly, 78.0 for solvent dielectric constant, 0.15 M for the salt concentration, and 300 K for the temperature). The last structural snapshot of each apo simulation was prepared by PDB2PQR 2.1 and was used as the input of calculations. Molecular graphics and electric fields were rendered by VMD.

Cell culture and transfection. C2C12, HEK293 and HeLa cells were cultured and maintained in Dulbecco's modified Eagle's medium (DMEM) supplemented with 10% fetal bovine serum (FBS) and high glucose (4.5 g/L) at 37° C. R-CatchER, G-CatchER2, G-CEPIA1er, R-CEPIA1er or R-CatchER with GFP-CaSR (wt and mutations) were transfected into cells using Lipofectamine 3000 (Life Technologies), following the manufacturer's instructions. Seed cells onto sterilized 22 mm×40 mm glass microscope slides in 6 cm dishes until about 70% confluency, the day of transfection. The next day, 2 μg of plasmid were mixed with transfection reagent in the reduced serum media Opti-MEM for 4-6 h at 37° C. The media was then replaced with 3 mL of fresh DMEM and incubated at 37° C. for 48 h.

Epifluorescence imaging of class C GPCR mediated ER Ca²⁺ dynamics using R-CatchER and Fura-2. HEK293 Cells transfected with R-CatchER and GFP-CaSR (wt and mutations) were incubated with Fura-2 for 30 mins at 37° C. then washed with 2 mL of physiological Ringer buffer (10 mM HEPES, 140 mM NaCl, 5 mM KCl, 1.2 mM MgCl₂, 1.8 mM CaCl₂) at pH 7.4). The coverslips were mounted on a bath chamber and placed on the stage of a Leica DM6100B inverted microscope with a Hamamatsu cooled EM-CCD camera and illuminated with a Till Polychrome V Xenon lamp. Cells were illuminated at 340 nm, 380 nm and 569 nm, in real-time, as cells were exposed to different concentrations of Ca²⁺, cinacalcet, Phe, TNCA, or NPS2143.

HILO imaging of R-CatchER, G-CatchER2, R-CEPIA1er, and G-CEPIA1er. Samples were mounted in a perfusion chamber and imaged using a customized optical microscope based on a Nikon TiE inverted microscope equipped with a 100×TIRF objective (N.A. 1.49, Nikon) and a highly sensitive electron multiplying charge-coupled device (EMCCD) camera (Andor Ixon Ultra 888). A fiber-coupled 488 nm or 561 nm laser (LBX-488/LCX-561, Oxxius) was first collimated and then focused to the back focal plane of the TIRF objective using an achromatic optical lens of 200 mm focal length (AC254-200-A, Thorlabs). To achieve HILO imaging, the incident angle of the excitation laser was adjusted to be slightly smaller than the critical angles at the cell-coverslip interface through translational movement the optical axis of the incident light beam by a motorized stage (SGSP-20-20, Sigma Koki). An efficient excitation volume of few micrometers was obtained under HILO illumination. A quad-band filter set (TRF89901v2, Chroma) was used for filtering out the fluorescence background. Fluorescence images of samples were recorded at 1 Hz as the concentration of ER Ca²⁺ was perturbed by perfusion of 3 μM Thapsigargin, 0.5 mM 4-cmc, 1 mM 4-cmc, 100 μM ATP, or 100 μM histamine.

Confocal imaging of R-CatchER. HeLa cells were transfected with R-CatchER two days before fixing. Cells were fixed with 3.7% Thermo Scientific™ Pierce™ 16% Formaldehyde (w/v), methanol-free, and permeabilized with 0.1% Triton X-100. Cells were then stained with ER-Tracker green (Invitrogen) and with ProLong gold antifade mountant with DAPI (Invitrogen) for staining the nucleus. Confocal imaging then was performed using a Zeiss LSM 700 confocal laser scanning microscope (CLSM).

Wide-field imaging in neuronal cultures. Primary neuronal cultures were generated from embryonic day 18 or postnatal day 0-1 mice and plated onto poly-D-lysine (Sigma) coated coverslips as previously described. Neurons were maintained in neuronal feeding media (Neurobasal media, ThermoFisher Scientific) containing 1% GlutaMAX (ThermoFisher Scientific), 2% B-27 (ThermoFisher Scientific), 0.002 mg/mL Gentamicin (Sigma) with or without 10 μM 5 fluoro 2-deoxyuridine (Sigma-Aldrich) and fed every 3-4 days via half neuronal feeding media exchanges. Neurons were transfected with plasmid(s) through either lipofection or electroporation. For lipofection, at 11-12 days in vitro, cells were transfected using Lipofectamine 2000 Reagent (ThermoFisher Scientific) with a modified protocol. For electroporation, dissociated cells in suspension were electroporated in a cuvette using the 4D-Nucleofector system (Lonza) following the manufacturer's instructions prior to plating in FBS-containing Neurobasal media without B-27. Full media exchange was performed the following day with serum- and antibiotics-free Neurobasal media.

Neurons were imaged between 12-15 days in vitro, using an inverted (Olympus IX71) or an upright (Scientifica HyperScope) wide-field fluorescence microscope equipped with an epi-fluorescence turret (Olympus), a scientific CMOS camera (Hamamatsu ORCA-Flash4.0 LT), a mercury lamp (Olympus) or an LED light source (CoolLED pE-300ultra), and an oil- (Olympus Uapo/340 40×/1.35NA) or water-immersion objective (Nikon CFI75 LWD 16×W 0.8NA), respectively. R-CatchER or jGCaMP7s was viewed using a TRITC (Chroma 41002) or FITC (Chroma 41001) fluorescence filter cube, respectively. Images were obtained every 5 s or 33.3 ms using Micro-Manager (the Vale lab, UCSF) at room temperature.

The external solution contained 150 mM NaCl, 3 mM KCl, 2 mM MgCl₂, 2 mM CaCl₂), 10 mM HEPES, and 20 mM glucose at a pH of 7.35. mGluR agonists were applied in the bath. Field electrical stimulation was applied with a stimulus isolator (World Precision Instruments A360) via an imaging chamber with two platinum wires (Warner Instruments RC-21BRFS). Trains (2 ms to 2 s at 50 HZ) of pulses (10 mA for 2 ms) were controlled by pClamp 10 software and Digidata 1440A data acquisition system (Molecular Devices). Imaging data were processed and analyzed using in-house and NeuroMatic (Jason Rothman) macros in Igor Pro 8 (WaveMetrics).

Plasmid extraction. Antibiotics positive agarose plates were streaked with Invitrogen™ MAX Efficiency™ DH5α competent cells with different mutants. These plates were incubated overnight at 37° C. Then tubes of 10 mL Fisher BioReagents™ LB Miller broth with antibiotics were inoculated with one colony each and put into a shaker overnight at 220 rpm and 37° C. The samples were centrifuged, and DNA extracted per QIAprep spin miniprep kit protocol.

Polymerase chain reaction (PCR). PCR site directed mutagenesis was performed using either G-Biosciences Pfu DNA polymerase or Sigma-Aldrich KOD DNA polymerase according to the manufacturer's instructions. Briefly, a pair of complementary primers were designed for generating each mutant with the mutation placed at the middle of the primers. The template DNA was amplified using these primers for 30 cycles in a polymerase chain reaction instrument (Techne). After digestion of the template DNA with New England Biolabs Dpn1, the amplified mutant DNA was transformed and amplified using Agilent XL10-Gold ultracompetent cells. All the DNA sequences were verified by Genewiz.

Agarose gel electrophoresis. The agarose gel for the PCR product was made using 50 mL of Thermo Scientific™ TAE Buffer (Tris-acetate-EDTA) at 1× concentration with 0.8% agarose. This mixture was heated for 90 seconds until boiled and fully dissolved. The mixture was then allowed to cool until warm to the touch. Then a 1:10,000 ratio of SYBR Safe DNA Gel Stain (10,000×DMSO) could be added to the mixture and poured into the UV transparent gel tray and left in the dark until solidified. The samples were run on agarose gel using gel electrophoresis at 80-120 V and imaged using UV light. PCR segments were extracted from the gels then were ligated with the template.

Statistics. Numbers in the text and error bars in the figures indicate mean±SEM. Student's T Tests or One-way ANOVA were used to determine the significant difference.

TABLE 1
Mutagenesis of G-CatchER, G-CatchER2, R-CatchER, and MCD1.

| | Mutations |
|---|---|
| EGFP | |
| G-CatchER | S147E/S202D/Q204E/F223E/T225E |
| G-CatchER2 | S30R/Y39N/S147D/S175G/S202D/Q204E/F223E/T225E |
| mApple | |
| R-CatchER | A145E/K198D/R216D |
| mCherry | |
| MCD1 | A145E/S147E/N196D/K198D/R216E |

TABLE 2
Biophysical properties of purified representative Ca²⁺ indicators.

| | Dynamic | K_{d} | Hill | kₒₙ | k_{off} | pKₐ | pKₐ | ε (apo) (×10⁴) | ε (holo) (×10⁴) | Φ | Φ |
|---|---|---|---|---|---|---|---|---|---|---|---|
| | Range | (μM) | coefficient | (M⁻¹s⁻¹) | (s⁻¹) | (apo) | (holo) | (M⁻¹ cm⁻¹) | (M⁻¹ cm⁻¹) | (apo) | (holo) |
| R-CatchER | 4.2 | 361 | 0.98 | >7 × 10⁶ | >2 × 10³ | 8.6 | 7.1 | 4 | 6 | 0.30 | 0.50 |
| G-CatchER | 1.9 | 230 | 0.94 | ~3.7 × 10⁶ | ~700 | 7.2 | 7.0 | 7 | 15 | 0.55 | 0.85 |
| G-CatchER2 | 3.9 | 1140 | 1.01 | — | — | 8.7 | 8.1 | 0.1 | 0.3 | 0.45 | 0.52 |
| R-CEPIA1er | 8.8 | 565 | 1.70 | 3.2 × 10⁵ | 183 | 8.9 | 6.5, 9.0 | 0.5 | 3.5 | 0.09 | 0.18 |
| G-CEPIA1er | 4.7 | 672 | 1.95 | 1.2 × 10⁵ | 81 | 8.7 | 8.0 | 0.3 | 1.0 | 0.19 | 0.40 |
| jGCaMP7s | 40.4 | 0.068 | 2.49 | 21.5 × 10⁶ | 2.87, 0.27 | 7.7 | 6.4 | 0.6 | 5.3 | 0.58 | 0.65 |
| GCaMP6s | 53.8 | 0.15 | 2.45 | 4.3 × 10⁶ | 0.69 | 7.5 | 6.0 | 0.2 | 7.0 | 0.41 | 0.64 |

TABLE 3
Attempts in creating mCherry based indicators.

| | Negatively | |
|---|---|---|
| | charged | |
| | residues | Fₘₐₓ/Fₘᵢₙ |
| mCherry A145E/N196D/K198D | 4 | 0.93 ± 0.02 |
| mCherry A145E/N196D/K198D/R216E | 5 | 1.05 ± 0.06 |
| mCherry | 6 | 1.13 ± 0.01 |
| A145E/S147E/N196D/K198D/R216E (MCD1) | | |

TABLE 4
List of calcium sensors with mutations.

| | | | | System size | | Duration |
|---|---|---|---|---|---|---|
| # | Protein | Chromophore | Ligand | # of a.a. (atoms) | PDB | (us) |
| 1 | R-Catch ER | CH6 | — | 217 (24,141) | 2H5O | 4.0 |
| 2 | R-CatchER | CH6 | Ca²⁺ | 217 (24,158) | 2H5O | 4.0 |
| 3 | G-CatchER | CRO | — | 228 (30,580) | 4L1I | 2.0 |
| 4 | G-CatchER | CRO | Ca²⁺ | 228 (30,585) | 4L1I | 2.0 |
| 5 | G-CatchER (N149E/E204D/E223D) | CRO | — | 228 (30,585) | 4L1I | 2.0 |
| 6 | G-CatchER (N149E/E204D/E223D) | CRO | Ca²⁺ | 228 (30,596) | 4L1I | 2.0 |
| 7 | G-CatchER (N149E/E204D) | CRO | — | 228 (30,570) | 4L1I | 2.0 |
| 8 | G-CatchER (N149E/E204D) | CRO | Ca²⁺ | 228 (30,578) | 4L1I | 2.0 |
| 9 | G-CatchER (E223D) | CRO | — | 228 (30,568) | 4L1I | 2.0 |
| 10 | G-CatchER (E223D) | CRO | Ca²⁺ | 228 (30,582) | 4L1I | 2.0 |
| 11 | G-CatchER (E204D) | CRO | — | 228 (30,577) | 4L1I | 2.0 |
| 12 | G-CatchER (E204D) | CRO | Ca²⁺ | 228 (30,582) | 4L1I | 2.0 |
| 13 | G-CatchER (E147D) | CRO | — | 228 (30,577) | 4L1I | 2.0 |
| 14 | G-CatchER (E147D) | CRO | Ca²⁺ | 228 (30,576) | 4L1I | 2.0 |
| 15 | G-CatchER (N149D) | CRO | — | 228 (30,570) | 4L1I | 2.0 |
| 16 | G-CatchER (N149D) | CRO | Ca²⁺ | 228 (30,572) | 4L1I | 2.0 |
| 17 | G-CatchER2 | CRO | — | 228 (30,569) | 4L1I | 2.0 |
| 18 | G-CatchER2 | CRO | Ca²⁺ | 228 (30,565) | 4L1I | 2.0 |
| 19 | MCD1 | CH6 | — | 218 (26,607) | 2H5Q | 2.0 |
| 20 | MCD1 | CH6 | Ca²⁺ | 218 (26,597) | 2H5Q | 2.0 |
| 21 | mApple | CH6 | — | 217 (24,117) | 2H5O | 4.0 |
| 22 | mApple | CH6 | Ca²⁺ | 217 (24,116) | 2H5O | 4.0 |
| 23 | mApple (K198D) | CH6 | — | 217 (24,118) | 2H5O | 4.0 |
| 24 | mApple (K198D) | CH6 | Ca²⁺ | 217 (24,120) | 2H5O | 4.0 |
| 25 | mApple (K198D/R216D) | CH6 | — | 217 (24,165) | 2H5O | 4.0 |
| 26 | mApple (K198D/R216D) | CH6 | Ca²⁺ | 217 (24,158) | 2H5O | 4.0 |
| 27 | mApple (A145E/K198D/R216E) | CH6 | — | 217 (24,150) | 2H5O | 4.0 |
| 28 | mApple (A145E/K198D/R216E) | CH6 | Ca²⁺ | 217 (24,164) | 2H5O | 4.0 |
| 29 | mApple (A145D/K198D/R216E) | CH6 | — | 217 (24,159) | 2H5O | 4.0 |
| 30 | mApple (A145D/K198D/R216E) | CH6 | Ca²⁺ | 217 (24,158) | 2H5O | 4.0 |
| 31 | mApple (A145E/E147D/K198D/R216E) | CH6 | — | 217 (24,153) | 2H5O | 4.0 |
| 32 | mApple (A145E/E147D/K198D/R216E) | CH6 | Ca²⁺ | 217 (24,152) | 2H5O | 4.0 |
| 33 | mApple (A145D/E147D/K198D/R216E) | CH6 | — | 217 (24,168) | 2H5O | 4.0 |
| 34 | mApple (A145D/E147D/K198D/R216E) | CH6 | Ca²⁺ | 217 (24,161) | 2H5O | 4.0 |
| 35 | mApple (A145E/K198D/R216E/E218D) | CH6 | — | 217 (24,144) | 2H5O | 4.0 |
| 36 | mApple (A145E/K198D/R216E/E218D) | CH6 | Ca²⁺ | 217 (24,146) | 2H5O | 4.0 |
| 37 | mApple (A145E/K198E/R216E) | CH6 | — | 217 (24,168) | 2H5O | 4.0 |
| 38 | mApple (A145E/K198E/R216E) | CH6 | Ca²⁺ | 217 (24,173) | 2H5O | 4.0 |
| 39 | EGFP | CRO | — | 226 (27,429) | 2YOG | 2.0 |
| 40 | mCherry | CH6 | — | 218 (26,617) | 2H5Q | 2.0 |

| | |
|---|---|
| R-CatchER | SEQ ID NO: 12 |
| R-CatchER | SEQ ID NO: 12 |
| G-CatchER | SEQ ID NO: 8 |
| G-CatchER (N149E/E204D/E223D) | SEQ ID NO: 49 |
| G-CatchER (N149E/E204D/E223D) | SEQ ID NO: 49 |
| G-CatchER (N149E/E204D) | SEQ ID NO: 50 |
| G-CatchER (N149E/E204D) | SEQ ID NO: 50 |
| G-CatchER (223D) | SEQ ID NO: 51 |
| G-CatchER (223D) | SEQ ID NO: 51 |
| G-CatchER (204D) | SEQ ID NO: 52 |
| G-CatchER (204D) | SEQ ID NO: 52 |
| G-CatchER (147D) | SEQ ID NO: 53 |
| G-CatchER (147D) | SEQ ID NO: 53 |
| G-CatchER (149D) | SEQ ID NO: 54 |
| G-CatchER (149D) | SEQ ID NO: 54 |
| G-CatchER2 | SEQ ID NO: 10 |
| G-CatchER2 | SEQ ID NO: 10 |
| MCD1 | SEQ ID NO: 32 |
| MCD1 | SEQ ID NO: 32 |
| mApple | SEQ ID NO: 11 |
| mApple | SEQ ID NO: 11 |
| mApple (K198D) | SEQ ID NO: 23 |
| mApple (K198D) | SEQ ID NO: 23 |
| mApple (K198D/R216D) | SEQ ID NO: 24 |
| mApple (K198D/R216D) | SEQ ID NO: 24 |
| mApple (A145E/K198D/R216E) | SEQ ID NO: 25 |
| mApple (A145E/K198D/R216E) | SEQ ID NO: 25 |
| mApple (A145D/K198D/R216E) | SEQ ID NO: 26 |
| mApple (A145D/K198D/R216E) | SEQ ID NO: 26 |
| mApple (A145E/E147D/K198D/R216E) | SEQ ID NO: 27 |
| mApple (A145E/E147D/K198D/R216E) | SEQ ID NO: 27 |
| mApple (A145D/E147D/K198D/R216E) | SEQ ID NO: 28 |
| mApple (A145D/E147D/K198D/R216E) | SEQ ID NO: 28 |
| mApple (A145E/K198D/R216E/E218D) | SEQ ID NO: 29 |
| mApple (A145E/K198D/R216E/E218D) | SEQ ID NO: 29 |
| mApple (A145E/K198E/R216E) | SEQ ID NO: 30 |
| mApple (A145E/K198E/R216E) | SEQ ID NO: 30 |
| EGFP | SEQ ID NO: 1 |
| mCherry | SEQ ID NO: 31 |

TABLE 5
Spectroscopic properties of EGFP and mApple variants.

| | Aₕₒₗₒ/Aₐₚₒ | Fₘₐₓ/Fₘᵢₙ |
|---|---|---|
| EGFP S147E/S202D/Q204E/F223E/T225E | 0.86 (488) | 1.90 |
| EGFP S147E/S202D/Q204E/F223D/T225E | 0.86 (488) | 1.26 |
| EGFP S147E/S202D/Q204D/F223E/T225E | 1.02 (488) | 1.20 |
| EGFP S147D/S202D/Q204E/F223E/T225E | 3.22 (488) | 4.12 |
| EGFP S147E/N149D/S202D/Q204E/F223E/T225E | 1.29 (488) | 1.88 |
| EGFP S147E/N149E/S202D/Q204D/F223E/T225E | 1.46 (488) | 1.50 |
| EGFP S147E/N149E/S202D/Q204D/F223D/T225E | 1.07 (488) | 1.23 |
| EGFP S30R/Y39N/S147D/S175G/S202D/Q204E/F223E/T225E (G-CatchER2) | 3.10 (488) | 3.91 |
| mApple | 1.07 (569) | 1.02 |
| mApple K198D | 1.41 (569) | 1.17 |
| mApple K198D/R216D | 2.21 (569) | 2.48 |
| mApple A145E/K198D/R216D (R-CatchER) | 4.33 (569) | 4.22 |
| mApple A145E/K198D/R216E | 1.60 (569) | 3.80 |
| mApple A145D/K198D/R216E | 1.57 (569) | 3.75 |
| mApple A145E/E147D/K198D/R216E | 1.66 (569) | 4.43 |
| mApple A145D/E147D/K198D/R216E | 1.84 (569) | 3.23 |
| mApple A145E/K198D/R216E/E218D | 1.59 (569) | 2.64 |
| mApple A145E/K198E/R216E | 1.04 (569) | 2.15 |

| | |
|---|---|
| EGFP S147E/S202D/Q204E/F223E/T225E (G-CatchER) | SEQ ID NO: 8 |
| EGFP S147E/S202D/Q204E/F223D/T225E | SEQ ID NO: 17 |
| EGFP S147E/S202D/Q204D/F223E/T225E | SEQ ID NO: 18 |
| EGFP S147D/S202D/Q204E/F223E/T225E | SEQ ID NO: 19 |
| EGFP S147E/N149D/S202D/Q204E/F223E/T225E | SEQ ID NO: 20 |
| EGFP S147E/N149E/S202D/Q204D/F223E/T225E | SEQ ID NO: 21 |
| EGFP S147E/N149E/S202D/Q204D/F223D/T225E | SEQ ID NO: 22 |
| EGFP S30R/Y39N/S147D/S175G/S202D/Q204E/F223E/T225E (G- | SEQ ID NO: 10 |
| CcatchER2) | |
| mApple | SEQ ID NO: 11 |
| mApple K198D | SEQ ID NO: 23 |
| mApple K198D/R216D | SEQ ID NO: 24 |
| mApple A145E/K198D/R216D (R-CatchER) | SEQ ID NO: 12 |
| mApple A145E/K198D/R216E | SEQ ID NO: 25 |
| mApple A145D/K198D/R216E | SEQ ID NO: 26 |
| mApple A145E/E147D/K198D/R216E | SEQ ID NO: 27 |
| mApple A145D/E147D/K198D/R216E | SEQ ID NO: 28 |
| mApple A145E/ K198D/R216E/E218D | SEQ ID NO: 29 |
| mApple A145E/ K198E/R216E | SEQ ID NO: 30 |

TABLE 6
In vitro properties of mApple variants

| | | Extinction coefficient | Fluorescence | |
|---|---|---|---|---|
| | pka | (M⁻¹ × cm⁻¹) | quantum yield | Brightness |
| mApple | 6.26 ± 0.23 | 7.5 × 10⁴ | 0.49 + 0.01 | 0.37 + 0.01 |
| mApple K198D (Apo) | 6.68 ± 0.09 | 5.9 × 10⁴ | 0.54 ± 0.01 | 0.32 ± 0.01 |
| mApple K198D (Holo) | 6.90 ± 0.12 | 6.9 × 10⁴ | 0.61 ± 0.03 | 0.42 ± 0.03 |
| mApple K198D/R216D (Apo) | 7.04 ± 0.11 | 3.8 × 10⁴ | 0.40 ± 0.01 | 0.15 ± 0.01 |
| mApple K198D/R216D (Holo) | 6.56 ± 0.11 | 6.5 × 10⁴ | 0.58 ± 0.01 | 0.37 ± 0.01 |
| mApple A145E/K198D/R216D (R-CatchER) (Apo) | 8.58 ± 0.11 | 4.4 × 10⁴ | 0.30 ± 0.02 | 0.13 ± 0.02 |
| mApple A145E/K198D/R216D (R-CatchER) (Holo) | 7.11 ± 0.10 | 6.0 × 10⁴ | 0.50 ± 0.03 | 0.30 ± 0.03 |

### Example 5. Rational Design and Evaluation of Mitochondria Calcium Indicator

### Introduction

The spatiotemporal pattern of Ca²⁺ dynamics is strongly influenced by Ca²⁺ buffering mechanisms, including the ER and Ca²⁺-binding proteins. In contrast to the classical view that mitochondria are static “power plants”, mitochondria are now recognized to play a key role in fine-tuning neuronal activity. This is accomplished partly by their ability to shape spatially localized domains of high Ca²⁺ increases, via the activity of the Ca²⁺ selective mitochondria uniporter. In addition to acting as a powerful intracellular Ca²⁺ buffering system, mitochondria can also act as a source of intracellular Ca²⁺ by releasing stored Ca²⁺. Importantly, mitochondria are highly dynamic and movable organelles, constantly undergoing morphological changes, including expansion and fragmentation (fusion/fission), which allow them to be dynamically recruited to areas of high Ca²⁺ activity, enhancing their Ca²⁺ buffering capabilities. Finally, it is now well accepted that mitochondria dysregulation of neuronal Ca²⁺ homeostasis contributes to numerous neurodegenerative and cardiovascular-related disorders, including heart failure, standing thus as a novel therapeutic target for the treatment of these prevalent diseases.

Here, this study initiated the design of mitochondria Ca²⁺ indicator using red fluorescent protein, mApple, with a single Ca²⁺ binding site. The results show increases in Ca²⁺ binding affinity through altering the H-bond network around the chromophore. We also characterized and applied one candidate in the mitochondria.

### Methods

Cloning, protein expression and purification. mApple variants were created by site-specific mutagenesis from parental scaffold mApple using Pfu DNA polymerase. All the DNAs for in vitro protein expression were subcloned into pRSETb with the BamH1 and EcoRI restriction sites. To target the proteins in the mitochondria lumen for cell imaging, the DNAs were subcloned into pCDNA3.1(+) vector by the same enzymes BamHI and EcoRI. Mitochondria targeting sequence COX VIII was inserted to the sequence in tandem. Proteins were expressed by Rosetta gami(DE3). Variants were expressed at 25° C. following the addition of 0.2 mM IPTG in Luria Bertani (LB) media with 50 mg/mL ampicillin. After centrifugation, cell pellets were re-suspended in 20-30 mL of lysis buffer (20 mM Tris, 100 mM NaCl, 0.1% Triton X-100, pH 8.0) and sonicated. The resulting lysate containing the protein of interest was centrifuged, and the supernatant was filtered and applied to a 5 mL Ni²⁺-NTA HiTrap™ HP chelating column (GE Healthcare) for HisTag purification using an imidazole gradient. To remove imidazole, pure protein fractions were concentrated to 1 mL, and buffer exchanged on a Superdex 200 gel filtration column (GE Healthcare) using 10 mM Tris pH 7.4 at 1 mL/min.

Plasmid extraction. Antibiotics positive agarose plates were streaked with Invitrogen™ MAX Efficiency™ DH5α competent cells with different mutants. These plates were incubated overnight at 37° C. Then tubes of 10 mL Fisher BioReagents™ LB Miller broth with antibiotics were inoculated with one colony each and put into a shaker overnight at 220 rpm and 37° C. The samples were centrifuged, and DNA extracted per QIAprep spin miniprep kit protocol.

Polymerase chain reaction (PCR). PCR site directed mutagenesis was performed using either G-Biosciences Pfu DNA polymerase according to the manufacturer's instructions. Briefly, a pair of complementary primers were designed for generating each mutant with the mutation placed at the middle of the primers. The template DNA was amplified using these primers for 30 cycles in a polymerase chain reaction instrument (Techne). After digestion of the template DNA with New England Biolabs Dpn1, the amplified mutant DNA was transformed and amplified using Agilent XL10-Gold ultracompetent cells. All the DNA sequences were verified by Genewiz.

Ca²⁺ binding assay. Fluorescence measurements of mApple variants with increasing Ca²⁺ concentrations were done in order to obtain the affinity of the sensor for Ca²⁺ in vitro. Samples of 10 μM mApple variants with 5 μM EGTA were prepared in triplicate in 1 mL volumes in 10 mM Tris, pH 7.4. The samples were placed in quartz fluorescence cuvettes, and metal ion was titrated into each sample, in a stepwise manner, using 0.1 M and 1 M metal stock solutions. The fluorescence response of the indicator to increasing Ca²⁺ concentrations was monitored using a fluorescence spectrophotometer (Photon Technology International, Canada) with the Felix32 fluorescence analysis software. The absorbance spectra before and after titration were obtained using a Shimadzu UV-1601 spectrophotometer.

Cell culture and transfection. HeLa cells were cultured and maintained in DMEM supplemented with 10% FBS and high glucose (4.5 g/L) at 37° C. Individual plasmids were transfected into cells using Lipofectamine 3000 (Life Technologies), following the manufacturer's instructions. Seed cells onto sterilized 22 mm×40 mm glass microscope slides in 6 cm dishes until about 70% confluency, the day of transfection. The next day, 2 μg of plasmid were mixed with transfection reagent in the reduced serum media Opti-MEM for 4-6 h at 37° C. The media was then replaced with 3 mL of fresh DMEM and incubated at 37° C. for 48 h.

Epifluorescence imaging of mitochondria Ca²⁺ dynamics. The coverslips with HeLa Cells transfected with mitochondria indicators were mounted on a bath chamber and placed on the stage of a Leica DM6100B inverted microscope with a Hamamatsu cooled EM-CCD camera and illuminated with a Till Polychrome V Xenon lamp. Cells were illuminated at 569 nm, in real-time. Fluorescence images of samples were recorded as the concentration of ER Ca²⁺ was perturbed by perfusion of 100 μM histamine.

### Results.

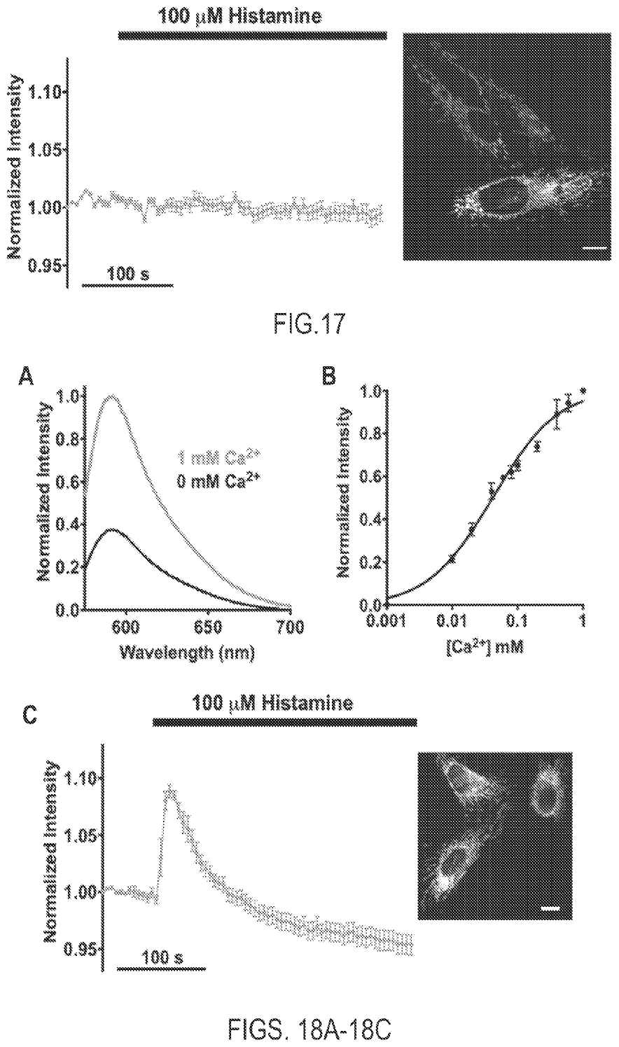

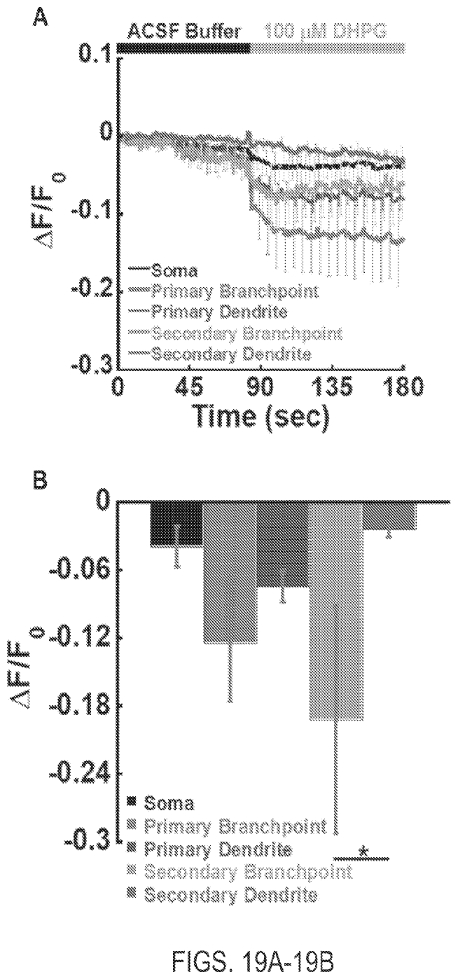

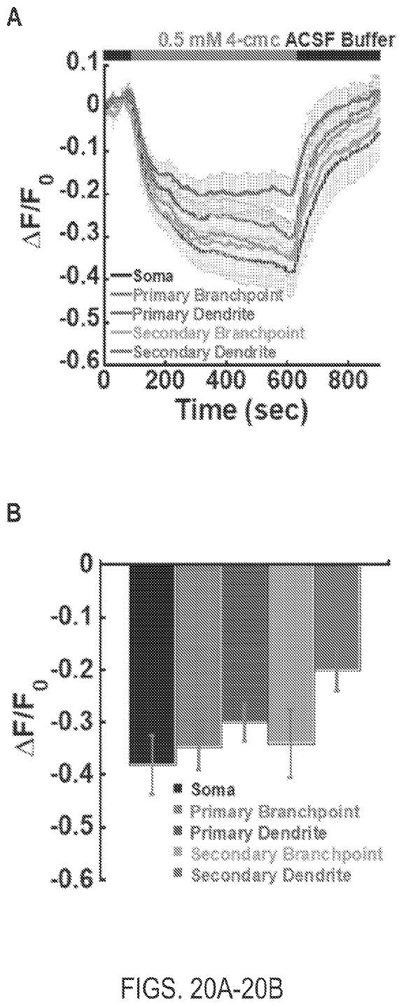

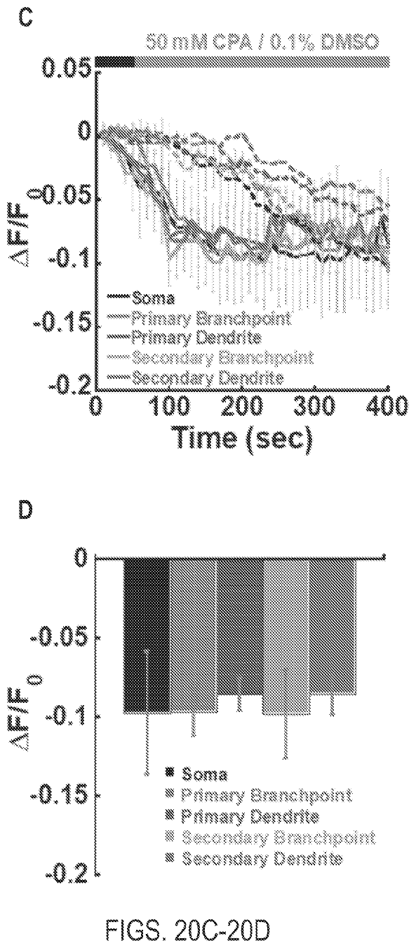

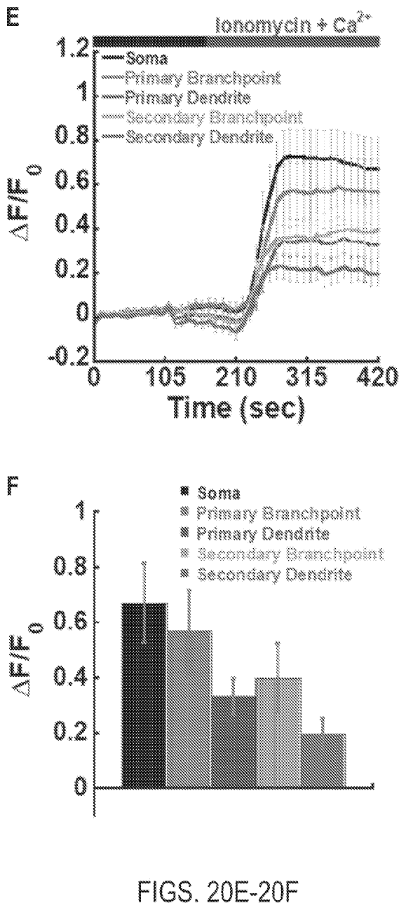

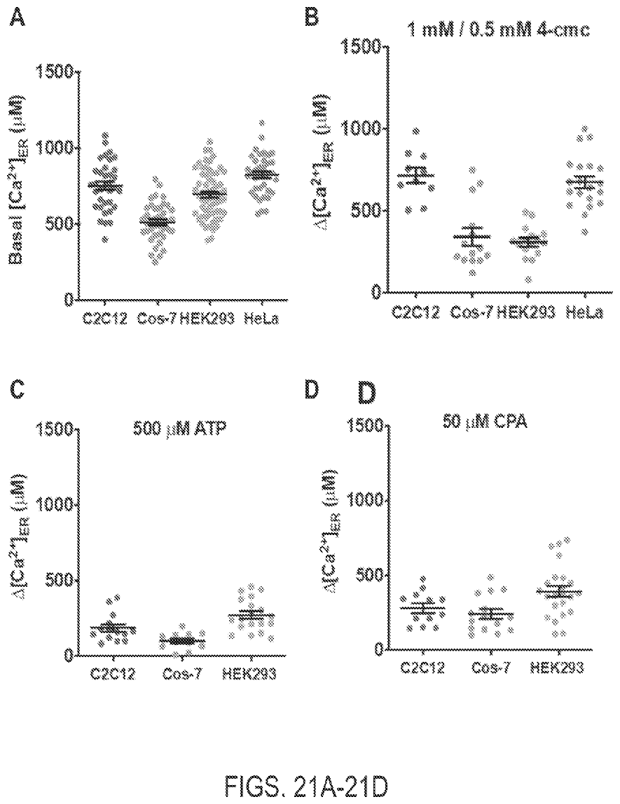

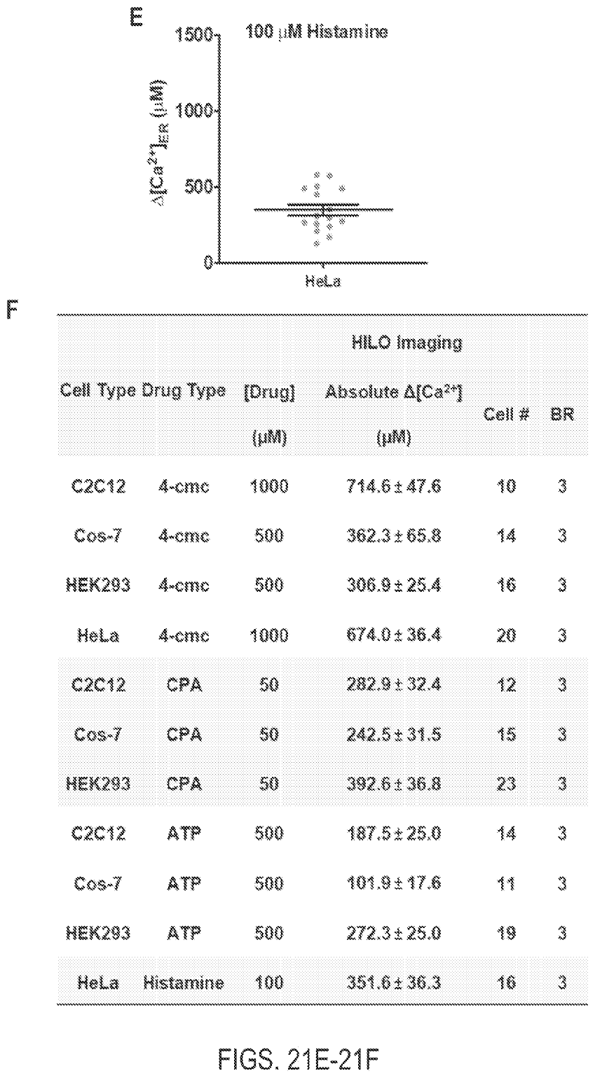

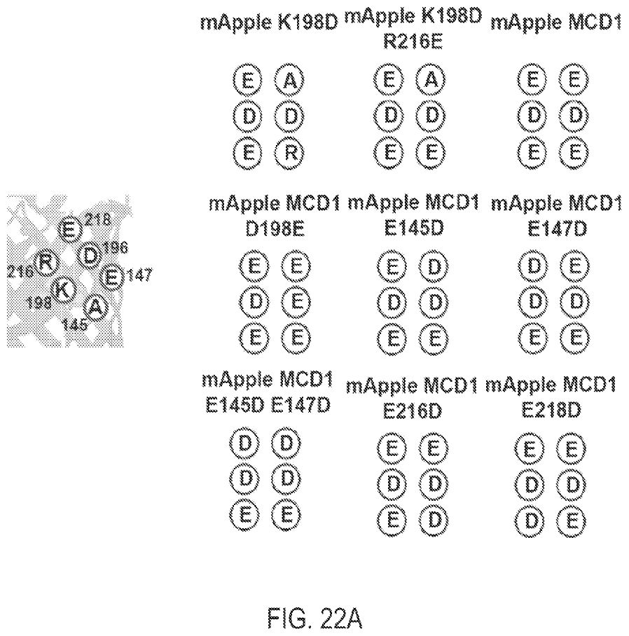

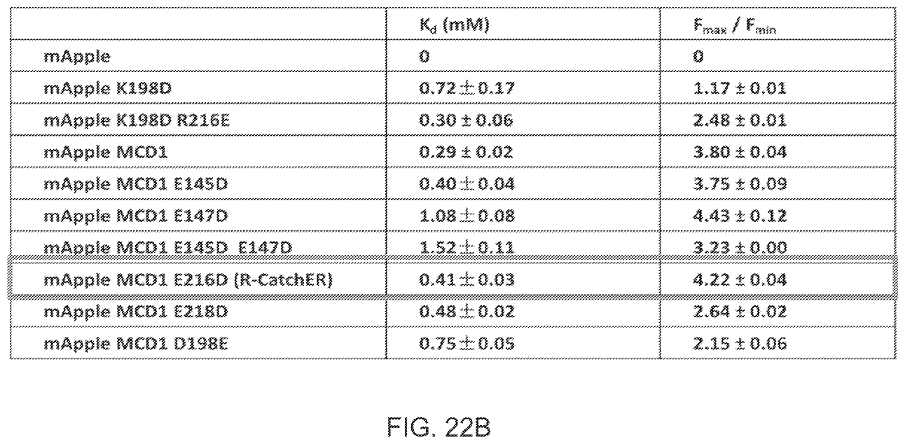

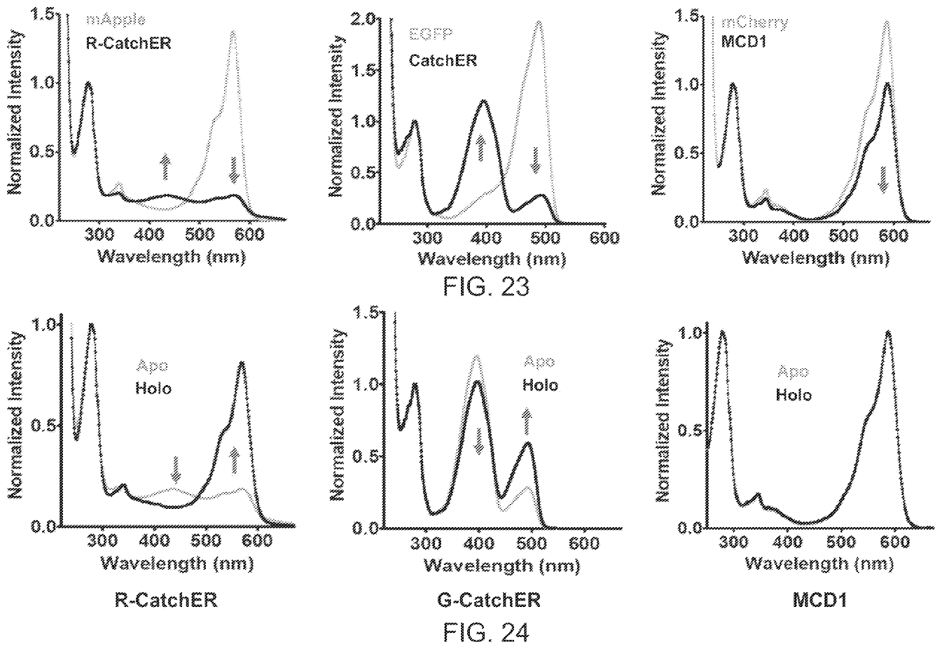

mApple A145E/K198D/R216E was chosen as the beginning point since it has the strongest binding affinity among all the mApple variants, with a K_{d} of 0.29±0.02 mM. Tandem 2×COX VIII mitochondria targeting sequence was inserted at the N-terminal of the mApple A145E/K198D/R216E, resulted in a successful expression of mApple A145E/K198D/R216E in mitochondria (FIG. 17). However, after applying 100 μM histamine to the HeLa cells transfected with mApple A145E/K198D/R216E, no fluorescence change was observed, indicating that the current binding affinity is too weak to be able to detect mitochondria Ca²⁺ dynamics (FIG. 17). CatchER and MCD15 variants were developed by altering the H-bond of the chromophore partially. Thus, this study aimed to increase the binding affinity of mApple variants by mutating residues contributed to the H-bond of the chromophore. It has been shown that Lys163 in mApple based R-GECO series Ca²⁺ indicators formed an ionic interaction with the phenolate oxygen of the chromophore. Thus, three mutations, K163Q, K163M, and K163L, were introduced into mApple A145E/K198D/R216E. One of the mutations, mApple A145E/K163L/K198D/R216E, showed significantly improved binding affinity, with a K_{d} of 54.3±9.6 μM. Additionally, after applying 100 μM histamine to the HeLa cells, mApple A145E/K163L/K198D/R216E showed nearly 10% fluorescence intensity increase, indicating its capacity in monitoring mitochondria Ca²⁺ dynamics (FIG. 18).

Mitochondria are primarily involved in cell survival and buffering intracellular Ca²⁺ signaling. Mitochondria prevent the intracellular overload by influx the cytosolic Ca²⁺ through MCU from either ER or extracellular environment. Loss of function of MCU causes abnormal mitochondrial Ca²⁺ dynamics, resulting in cell death and neurodegenerative diseases. Thus, monitoring mitochondrial Ca²⁺ is critical for cell function, and growing attention has been made to the development of mitochondrial Ca²⁺ indicators. It has been shown that the resting Ca²⁺ level in mitochondria is at a similar level with cytosolic Ca²⁺ concentration (<100 nM). Thus, some cytosolic Ca²⁺ indicators have been successfully used to detect mitochondrial Ca²⁺ dynamics. However, mitochondrial Ca²⁺ concentration can reach up to ˜100 mM after certain types of stimulation. It is also important to develop mitochondrial GECIs possessing low Ca²⁺ affinity to report those events.

It's promising that mApple A145E/K163L/K198D/R216E showed a nearly 10% fluorescence intensity increase after applying 100 μM histamine, indicating its capacity in monitoring mitochondria Ca²⁺ dynamics.

### Example 6. Catch Derivatives for Micro/Nanodomain Ca2+ Responses with Targeting Capability to Subcellular Organelles (e.g. ER and Mitochondria) and Channels/Receptors (e.g. TRP, NMDA, and AMPA)

The CatchER⁺ and R-CatchER can specifically report rapid local ER Ca²⁺ dynamics in various cell types with optimized chromophore folding at ambient temperatures. Having fast kinetics, CatchER⁺ and R-CatchER was able to record the sarcoplasmic reticulum (SR) luminal Ca²⁺ in flexor digitorum brevis (FDB) muscle fibers during voltage stimulation, successfully determined decreased SR Ca²⁺ release in aging mice and reported changes in ER-mediated Ca²⁺ release upon stimulation in primary hippocampal neurons.

Gateway multisite recombineering has been used to generate inducible CatchER⁺ transgenic strains in Drosophila melanogaster for in vivo neural cell-type specific microdomain targeting. Multiple CatchER⁺ transgenic lines compatible with widely used binary expression systems (Gal4/UAS; LexA LexAop; QF QUAS) have been engineered in order to capitalize on the wealth of genetic tools available in Drosophila for cell- and tissue-type specific gene expression. ER targeting efficiency and specificity was confirmed in Drosophila multidendritic (md) sensory neurons in combination with ER-specific reporters revealing robust expression of the CatchER⁺ sensor in ER networks located within the soma and at satellite locations on dendrites. Therefore, the results herein and the previous publications demonstrate how this approach can circumvent limitations associated with current GECIs based on endogenous Ca²⁺ binding proteins.

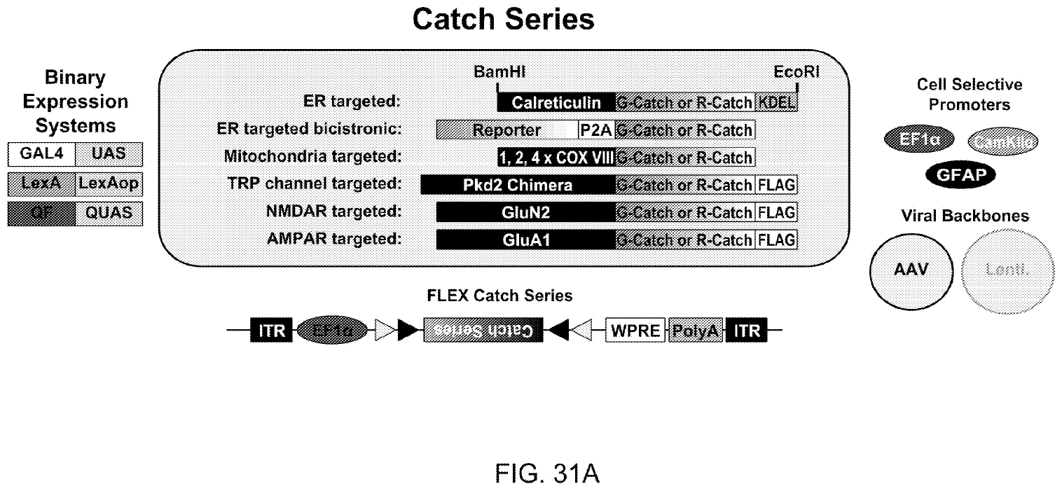

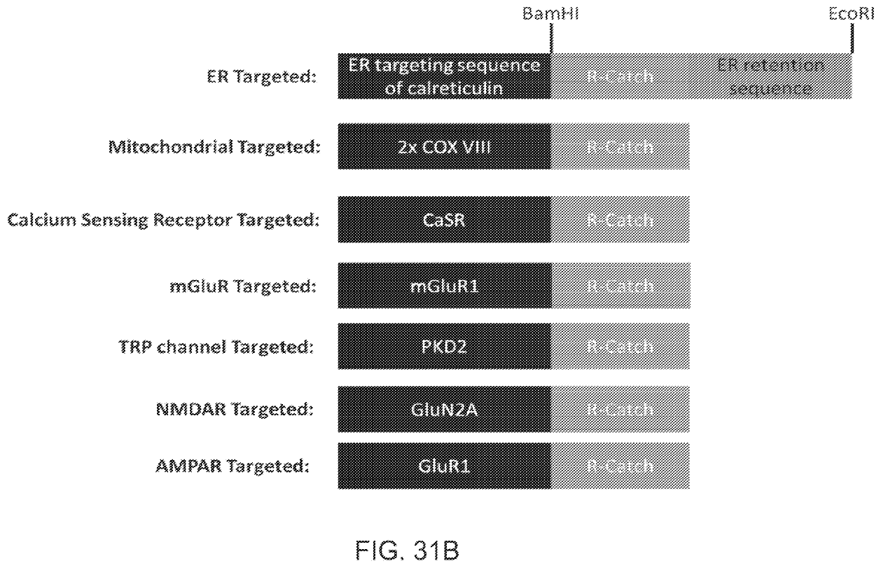

Catch derivatives (G-Catch and R-Catch) for micro/nanodomain Ca²⁺ responses with targeting capability to subcellular organelles (e.g. ER and mitochondria) and channels/receptors (e.g. TRP, NMDA, and AMPA). Targeting efficiency and specificity of the novel sensors were verified using in vitro (mouse primary neurons via transient transfection) followed by selected in vivo confirmation (Drosophila neurons via binary expression system) using various imaging modalities. Optimized and verified sensors were selected for multiplex compatible approaches that include combinatorial binary expression systems and CRE-targeted AAV/Lentiviral transduction systems for in vivo mammalian studies. The Catch series sensors targeting subcellular organelles (e.g. ER and mitochondria) and channels/receptor (Calcium sensing receptor (CaSR), mGluR receptor, TRP, NMDA, and AMPA (FIG. 31) were expressed in mouse primary neuronal cells as previously described to verify the efficiency, specificity. Ca²⁺ binding affinity, dynamic range of Ca²⁺ dependent fluorescence/lifetime changes and kinetic responses. Their targeting capability were validated via immunostaining aid/or live imaging using TIRF/HILO/confocal/2-photon microscopy as appropriate. Ca²⁺ binding affinities (K_{d}) were determined using the established protocol for the in situ K_{d} measurement and calibration in neurons. Kinetics were calculated by fitting the response curves in combination with electrophysiological stimulation. In the case of nanodomain sensors fused to channels/receptors, preservation of function by electrophysiological comparisons of stimulus-evoked currents in cultured cells (e.g., HEK293 or primary neurons) expressing the channel/receptor in the presence or absence of the Catch sensor and under ionic gradient conditions designed to promote elevation of intracellular Ca²⁺ were investigated.

For viral transduction in vivo with select targeting efficiency, sensors with ideal attributes were generated as viruses and validated for expression in vivo. Briefly, the selected Ca²⁺ sensor series generated above were cloned into lentiviral and adeno-associated virus (AAV) vector backbones for in vivo expression in mammals using different promotors to target the sensors to select brain regions. Specifically, the CaMKIIα promoter can provide select expression in principle pyramidal cell types (excitatory neurons), the CAG/EF1α promoter can generate a broad cellular expression in the brain and the GFAP promoter can allow selective expression in glial populations. Moreover, a bicistronic element (P2A) was incorporated to allow for simultaneous expression of a reporter (e.g. tdTomato) with these Catch variants, thus providing an intrinsic control for sensor dynamics and the ability to co-register cell morphology. Finally, DIO-AAV FLEX Catch variants were generated, which allows capitalizing on selective expression of Catch series in the vast number of CRE-positive transgenic mammalian animal lines. Viral expression of these sensors were validated in acute brain slices via stereotactically guided injections into the dorsal CA1 hippocampal region of mice. As an example, following viral injection of CatchER⁺, mGluR-mediated ER Ca²⁺ release was triggered by acute treatment of hippocampal slices with the group I mGluR agonist DHPG. Corresponding fluorescence changes were measured using 2-photon microscopy. For the in vitro and in vivo studies above, dynamics were compared with existing Ca²⁺ indicators such as CEPIA1er, low affinity GCaMPs and GCaMPer.

Based upon the in vitro validation studies, Catch derivatives were optimized for generation of selected micro/nanodomain targeting transgenic sensor strains in Drosophila (EGFP/mCherry-tagged versions), the proven recombineering strategy was used to engineer the binary expression system compatible transgenes focusing on the generation of transgenic Catch strains for microdomain (ER/mitochondria) and nanodomain (TRPP channel Pkd2) in vivo analyses (FIG. 31). Expression of transgenic microdomain sensors can be verified in multiple neuronal cell types at both larval and adult stages (e.g., multidendritic sensory neurons; motorneurons, and visual system neurons) to establish generalizable utility. To validate targeting specificity, these sensors were expressed in combination with already existing organelle-specific transgenic reporters in the aforementioned neuronal subtypes. Co-localization and distribution analyses were conducted using live cell imaging of fluorescently tagged reporters as well as by immunohistochemistry utilizing relevant microscopy modalities (e.g. confocal/TIRF/HILO/2-photon).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES
(EGFP, long)
SEQ ID NO: 1
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNSHNVYIMADKQKNGIKVNFKIRHNIEDGSV
QLADHYQQNTPIGDGPVLLPDNHYLSTQSALSKDPNEKRDHMVLLEFVTAAGITLGMDE
LYK
(G-CatchER, long)
SEQ ID NO: 2
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHNVYITADKQKNGIKANFKIRHNIEDGSV
QLADHYQQNTPIGDGPVLLPDNHYLDTESALSKDPNEKRDHMVLLEEVEAAGITLGMDE
LYK
(G-CatchER+, long)
SEQ ID NO: 3
MVSKGEELFTGVVPILVELDGDVNGHKFSVRGEGEGDATNGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHNVYITADKQKNGIKANFKIRHNIEDGGV
QLADHYQQNTPIGDGPVLLPDNHYLDTESALSKDPNEKRDHMVLLEEVEAAGITLGMDE
LYK
(G-CatchER2, long)
SEQ ID NO: 4
MVSKGEELFTGVVPILVELDGDVNGHKFSVRGEGEGDATNGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNDHNVYITADKQKNGIKANFKIRHNIEDGGV
QLADHYQQNTPIGDGPVLLPDNHYLDTESALSKDPNEKRDHMVLLEEVEAAGITLGMDE
LYK
(mApple, long)
SEQ ID NO: 5
MVSKGEENNMAIIKEFMRFKVHMEGSVNGHEFEIEGEGEGRPYEAFQTAKLKVTKGGPL
PFAWDILSPQFMYGSKVYIKHPADIPDYFKLSFPEGFRWERVMNFEDGGIIHVNQDSSL
QDGVFIYKVKLRGTNFPSDGPVMQKKTMGWEASEERMYPEDGALKSEIKKRLKLKDGGH
YAAEVKTTYKAKKPVQLPGAYIVDIKLDIVSHNEDYTIVEQYERAEGRHSTGGMDELYK
(R-CatchER, long)
SEQ ID NO: 6
MVSKGEENNMAIIKEFMRFKVHMEGSVNGHEFEIEGEGEGRPYEAFQTAKLKVTKGGPL
PFAWDILSPQFMYGSKVYIKHPADIPDYFKLSFPEGFRWERVMNFEDGGIIHVNQDSSL
QDGVFIYKVKLRGTNFPSDGPVMQKKTMGWEESEERMYPEDGALKSEIKKRLKLKDGGH
YAAEVKTTYKAKKPVQLPGAYIVDIDLDIVSHNEDYTIVEQYEDAEGRHSTGGMDELYK
(EGFP, short)
SEQ ID NO: 7
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNSHNVYIMADKQKNGIKVNFKIRHNIEDGSVQ
LADHYQQNTPIGDGPVLLPDNHYLSTQSALSKDPNEKRDHMVLLEFVTAAGITLGMDEL
YK
(CatchER, short, EGFP S147E/S202D/Q204E/F223E/
T225E in Table 5)
SEQ ID NO: 8
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHNVYITADKQKNGIKANFKIRHNIEDGSVQ
LADHYQQNTPIGDGPVLLPDNHYLDTESALSKDPNEKRDHMVLLEEVEAAGITLGMDEL
YK
(G-CatchER+, short)
SEQ ID NO: 9
VSKGEELFTGVVPILVELDGDVNGHKFSVRGEGEGDATNGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHNVYITADKQKNGIKANFKIRHNIEDGGVQ
LADHYQQNTPIGDGPVLLPDNHYLDTESALSKDPNEKRDHMVLLEEVEAAGITLGMDEL
YK
(G-CatchER2, short; EGFP S30R/Y39N/S147D/S175G/
S202D/Q204E/F223E/T225E (G-CcatchER2) in Table 5)
SEQ ID NO: 10
VSKGEELFTGVVPILVELDGDVNGHKFSVRGEGEGDATNGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNDHNVYITADKQKNGIKANFKIRHNIEDGGVQ
LADHYQQNTPIGDGPVLLPDNHYLDTESALSKDPNEKRDHMVLLEEVEAAGITLGMDEL
YK
(mApple, short; mApple in Table 5)
SEQ ID NO: 11
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEASEERMYPEDGALKSEIKKRLKLKDGGHYAAEV
KTTYKAKKPVQLPGAYIVDIKLDIVSHNEDYTIVEQYERAEGRHSTGGMDELYK
(R-CatchER, short; mApple A145E/K198D/R216D
(R-CatchER) in Table 5)
SEQ ID NO: 12
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEESEERMYPEDGALKSEIKKRLKLKDGGHYAAEV
KTTYKAKKPVQLPGAYIVDIDLDIVSHNEDYTIVEQYEDAEGRHSTGGMDELYK
(G-CatchER2 (long) DNA Sequence)
SEQ ID NO: 13
ATGGTGAGCAAGGGCGAGGAGCTGTTCACCGGGGTGGTGCCCATCCTGGTCGAGCTGGA
CGGCGACGTAAACGGCCACAAGTTCAGCGTGCGCGGCGAGGGCGAGGGCGATGCCACCA
ACGGCAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACCACAT
GAAGCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAACATCCT
GGGGCACAAGCTGGAGTACAACTACAACGACCACAACGTCTATATCACGGCCGACAAGC
AGAAGAACGGCATCAAGGCGAACTTCAAGATCCGCCACAACATCGAGGACGGCGGCGTG
CAGCTCGCCGACCACTACCAGCAGAACACCCCCATCGGCGACGGCCCCGTGCTGCTGCC
CGACAACCACTACCTGGACACCGAATCCGCCCTGAGCAAAGACCCCAACGAGAAGCGCG
ATCACATGGTCCTGCTGGAGGAGGTGGAGGCCGCCGGGATCACTCTCGGCATGGACGAG
CTGTACAAG
(R-CatchER (long) DNA Sequence)
SEQ ID NO: 14
ATGGTGAGCAAGGGCGAGGAGAATAACATGGCCATCATCAAGGAGTTCATGCGCTTCAA
GGTGCACATGGAGGGCTCCGTGAACGGCCACGAGTTCGAGATCGAGGGCGAGGGCGAGG
GCCGCCCCTACGAGGCCTTTCAGACCGCTAAGCTGAAGGTGACCAAGGGTGGCCCCCTG
CCCTTCGCCTGGGACATCCTGTCCCCTCAGTTCATGTACGGCTCCAAGGTCTACATTAA
GCACCCAGCCGACATCCCCGACTACTTCAAGCTGTCCTTCCCCGAGGGCTTCAGGTGGG
AGCGCGTGATGAACTTCGAGGACGGCGGCATTATTCACGTTAACCAGGACTCCTCCCTG
CAGGACGGCGTGTTCATCTACAAGGTGAAGCTGCGCGGCACCAACTTCCCCTCCGACGG
CCCCGTAATGCAGAAGAAGACCATGGGCTGGGAGGAGTCCGAGGAGCGGATGTACCCCG
AGGACGGCGCCCTGAAGAGCGAGATCAAGAAGAGGCTGAAGCTGAAGGACGGCGGCCAC
TACGCCGCCGAGGTCAAGACCACCTACAAGGCCAAGAAGCCCGTGCAGCTGCCCGGCGC
CTACATCGTCGACATCGACTTGGACATCGTGTCCCACAACGAGGACTACACCATCGTGG
AACAGTACGAGGACGCCGAGGGCCGCCACTCCACCGGCGGCATGGACGAGCTGTACAAG
SEQ ID NO: 15
KDEL
SEQ ID NO: 16
MLLSVPLLLGLLGLAAAD
EGFP S147E/S202D/Q204E/F223D/T225E
SEQ ID NO: 17
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHNVYITADKQKNGIKANFKIRHNIEDGSVQ
LADHYQQNTPIGDGPVLLPDNHYLDTESALSKDPNEKRDHMVLLEDVEAAGITLGMDEL
YK,
EGFP S147E/S202D/Q204D/F223E/T225E
SEQ ID NO: 18
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHNVYITADKQKNGIKANFKIRHNIEDGSVQ
LADHYQQNTPIGDGPVLLPDNHYLDTDSALSKDPNEKRDHMVLLEEVEAAGITLGMDEL
YK,
EGFP S147D/S202D/Q204E/F223E/T225E
SEQ ID NO: 19
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNDHNVYITADKQKNGIKANFKIRHNIEDGSVQ
LADHYQQNTPIGDGPVLLPDNHYLDTESALSKDPNEKRDHMVLLEEVEAAGITLGMDEL
YK,
EGFP S147E/N149D/S202D/Q204E/F223E/T225E
SEQ ID NO: 20
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHDVYITADKQKNGIKANFKIRHNIEDGSVQ
LADHYQQNTPIGDGPVLLPDNHYLDTESALSKDPNEKRDHMVLLEEVEAAGITLGMDEL
YK,
EGFP S147E/N149E/S202D/Q204D/F223E/T225E
SEQ ID NO: 21
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHEVYITADKQKNGIKANFKIRHNIEDGSVQ
LADHYQQNTPIGDGPVLLPDNHYLDTDSALSKDPNEKRDHMVLLEEVEAAGITLGMDEL
YK,
EGFP S147E/N149E/S202D/Q204D/F223D/T225E
SEQ ID NO: 22
VSKGEELFTGVVPILVELDGDVNGHKFSVSGEGEGDATYGKLTLKFICTTGKLPVPWPT
LVTTLTYGVQCFSRYPDHMKQHDFFKSAMPEGYVQERTIFFKDDGNYKTRAEVKFEGDT
LVNRIELKGIDFKEDGNILGHKLEYNYNEHEVYITADKQKNGIKANFKIRHNIEDGSVQ
LADHYQQNTPIGDGPVLLPDNHYLDTDSALSKDPNEKRDHMVLLEDVEAAGITLGMDEL
YK,
mApple K198D
SEQ ID NO: 23
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEASEERMYPEDGALKSEIKKRLKLKDGGHYAAEV
KTTYKAKKPVQLPGAYIVDIDLDIVSHNEDYTIVEQYERAEGRHSTGGMDELYK,
mApple K198D/R216D
SEQ ID NO: 24
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEASEERMYPEDGALKSEIKKRLKLKDGGHYAAEV
KTTYKAKKPVQLPGAYIVDIDLDIVSHNEDYTIVEQYEDAEGRHSTGGMDELYK,
mApple A145E/K198D/R216E
SEQ ID NO: 25
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEESEERMYPEDGALKSEIKKRLKLKDGGHYAAEV
KTTYKAKKPVQLPGAYIVDIDLDIVSHNEDYTIVEQYEEAEGRHSTGGMDELYK,
mApple A145D/K198D/R216E
SEQ ID NO: 26
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEDSEERMYPEDGALKSEIKKRLKLKDGGHYAAEV
KTTYKAKKPVQLPGAYIVDIDLDIVSHNEDYTIVEQYEEAEGRHSTGGMDELYK,
mApple A145E/E147D/K198D/R216E
SEQ ID NO: 27
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEESDERMYPEDGALKSEIKKRLKLKDGGHYAAEV
KTTYKAKKPVQLPGAYIVDIDLDIVSHNEDYTIVEQYEEAEGRHSTGGMDELYK,
mApple A145D/E147D/K198D/R216E
SEQ ID NO: 28
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEDSDERMYPEDGALKSEIKKRLKLKDGGHYAAEV
KTTYKAKKPVQLPGAYIVDIDLDIVSHNEDYTIVEQYEEAEGRHSTGGMDELYK,
mApple A145E/K198D/R216E/E218D
SEQ ID NO: 29
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEESEERMYPEDGALKSEIKKRLKLKDGGHYAAEV
KTTYKAKKPVQLPGAYIVDIDLDIVSHNEDYTIVEQYEEADGRHSTGGMDELYK;
mApple A145E/K198E/R216E
SEQ ID NO: 30
EENNMAIIKEFMRFKVHMEGSVNGHEFEIEGEGEGRPYEAFQTAKLKVTKGGPLPFAWD
ILSPQFMYGSKVYIKHPADIPDYFKLSFPEGFRWERVMNFEDGGIIHVNQDSSLQDGVF
IYKVKLRGTNFPSDGPVMQKKTMGWEESEERMYPEDGALKSEIKKRLKLKDGGHYAAEV
KTTYKAKKPVQLPGAYIVDIELDIVSHNEDYTIVEQYEEAEGRHSTGGMDELYK;
Additional sequences:
MCherry
SEQ ID NO: 31
EEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEF
IYKVKLRGTNFPSDGPVMQKKTMGWEASSERMYPEDGALKGEIKQRLKLKDGGHYDAEV
KTTYKAKKPVQLPGAYNVNIKLDITSHNEDYTIVEQYERAEGRHSTGGMDELYK;
mCherry A145E/S147E/N196D/K198D/R216E (MCD1)
SEQ ID NO: 32
EEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEF
IYKVKLRGTNFPSDGPVMQKKTMGWEESEERMYPEDGALKGEIKQRLKLKDGGHYDAEV
KTTYKAKKPVQLPGAYNVDIDLDITSHNEDYTIVEQYEEAEGRHSTGGMDELYK;
Mitochondria sensor from Example 5:
SEQ ID NO: 33
MVSKGEENNMAIIKEFMRFKVHMEGSVNGHEFEIEGEGEGRPYEAFQTAKLKVTKGGPL
PFAWDILSPQFMYGSKVYIKHPADIPDYFKLSFPEGFRWERVMNFEDGGIIHVNQDSSL
QDGVFIYKVKLRGTNFPSDGPVMQKKTMGWEESEERMYPEDGALKSEIKLRLKLKDGGH
YAAEVKTTYKAKKPVQLPGAYIVDIDLDIVSHNEDYTIVEQYEDAEGRHSTGGMDELY
K;
(Mitochondria targeting sequence/mitochondrial
COX VIII)
SEQ ID NO: 34
MLCQQMIRTTAKRSSNIMTRPIIMKRSVHFKDGVYENIPFKVKGRKTPYALSHFGFFAI
GFAVPFVACYVQLKKSGAF
Sequences from FIG. 31:
(ER targeting (R-CatchER), the underlined
represents the R-CatchER sequence and the
bolded represents the ER signaling)
SEQ ID NO: 35
MLLSVPLLLGLLGLAAADGDPATMVSKGEENNMAIIKEFMRFKVHMEGSVNGHEFEIEG
EGEGRPYEAFQTAKLKVTKGGPLPFAWDILSPQFMYGSKVYIKHPADIPDYFKLSFPEG
FRWERVMNFEDGGIIHVNQDSSLQDGVFIYKVKLRGTNFPSDGPVMQKKTMGWEESEER
MYPEDGALKSEIKKRLKLKDGGHYAAEVKTTYKAKKPVQLPGAYIVDIDLDIVSHNEDY
TIVEQYEDAEGRHSTGGMDELYKKDEL
(Mitochondria targeting; the bolded represents
the two times mitochondrial COX VIII, the
underlined represents the mitochondrial sensor)
SEQ ID NO: 36
MLCQQMIRTTAKRSSNIMTRPIIMKRSVHFKDGVYENIPFKVKGRKTPYALSHFGFFAI
GFAVPFVACYVQLKKSGAFMLCQQMIRTTAKRSSNIMTRPIIMKRSVHFKDGVYENIPF
KVKGRKTPYALSHFGFFAIGFAVPFVACYVQLKKSGAFMVSKGEENNMAIIKEFMRFKV
HMEGSVNGHEFEIEGEGEGRPYEAFQTAKLKVTKGGPLPFAWDILSPQFMYGSKVYIKH
PADIPDYFKLSFPEGFRWERVMNFEDGGIIHVNQDSSLQDGVFIYKVKLRGTNFPSDGP
VMQKKTMGWEESEERMYPEDGALKSEIKLRLKLKDGGHYAAEVKTTYKAKKPVQLPGAY
IVDIDLDIVSHNEDYTIVEQYEEAEGRHSTGGMDELYK
(Calcium Sensing Receptor targeting; bolded
represents CaSR, underlined represents R-CatchER)
SEQ ID NO: 37
MAFYSCCWVLLALTWHTSAYGPDQRAQKKGDIILGGLFPIHFGVAAKDQDLKSRPESVE
CIRYNFRGFRWLQAMIFAIEEINSSPALLPNLTLGYRIFDTCNTVSKALEATLSFVAQN
KIDSLNLDEFCNCSEHIPSTIAVVGATGSGVSTAVANLLGLFYIPQVSYASSSRLLSNK
NQFKSFLRTIPNDEHQATAMADIIEYFRWNWVGTIAADDDYGRPGIEKFREEAEERDIC
IDFSELISQYSDEEEIQHVVEVIQNSTAKVIVVFSSGPDLEPLIKEIVRRNITGKIWLA
SEAWASSSLIAMPQYFHVVGGTIGFALKAGQIPGFREFLKKVHPRKSVHNGFAKEFWEE
TFNCHLQEGAKGPLPVDTFLRGHEESGDRFSNSSTAFRPLCTGDENISSVETPYIDYTH
LRISYNVYLAVYSIAHALQDIYTCLPGRGLFINGSCADIKKVEAWQVLKHLRHLNFTNN
MGEQVTFDECGDLVGNYSIINWHLSPEDGSIVFKEVGYYNVYAKKGERLFINEEKILWS
GFSREPLTFVLSVLQVPFSNCSRDCLAGTRKGIIEGEPTCCFECVECPDGEYSDETDAS
ACNKCPDDFWSNENHTSCIAKEIEFLSWTEPFGIALTLFAVLGIFLTAFVLGVFIKFRN
TPIVKATNRELSYLLLFSLLCCFSSSLFFIGEPQDWTCRLRQPAFGISFVLCISCILVK
TNRVLLVFEAKIPTSFHRKWWGLNLQFLLVFLCTFMQIVICVIWLYTAPPSSYRNQELE
DEIIFITCHEGSLMALGFLIGYTCLLAAICFFFAFKSRKLPENFNEAKFITFSMLIFFI
VWISFIPAYASTYGKFVSAVEVIAILAASFGLLACIFFNKIYIILFKPSRNTIEEVRCS
TAAHAFKVAARATLRRSNVSRKRSSSLGGSTGSTPSSSISSKSNSEDPFPQPERQKQQQ
PLALTQQEQQQQPLTLPQQQRSQQQPRCKQKVIFGSGTVTFSLSFDEPQKNAMAHRNST
HQNSLEAQKSSDTLTRHQPLLPLQCGETDLDLTVQETGLQGPVGGDQRPEVEDPEELSP
ALVVSSSQSFVISGGGSTVTENVVNSMVSKGEENNMAIIKEFMRFKVHMEGSVNGHEFE
IEGEGEGRPYEAFQTAKLKVTKGGPLPFAWDILSPOFMYGSKVYIKHPADIPDYFKLSF
PEGFRWERVMNFEDGGIIHVNQDSSLQDGVFIYKVKLRGTNFPSDGPVMQKKTMGWEES
EERMYPEDGALKSEIKKRLKLKDGGHYAAEVKTTYKAKKPVOLPGAYIVDIDLDIVSHN
EDYTIVEQYEDAEGRHSTGGMDELYK
(CaSR targeting)
SEQ ID NO: 38
MAFYSCCWVLLALTWHTSAYGPDQRAQKKGDIILGGLFPIHFGVAAKDQDLKSRPESVE
CIRYNFRGFRWLQAMIFAIEEINSSPALLPNLTLGYRIFDTCNTVSKALEATLSFVAQN
KIDSLNLDEFCNCSEHIPSTIAVVGATGSGVSTAVANLLGLFYIPQVSYASSSRLLSNK
NQFKSFLRTIPNDEHQATAMADIIEYFRWNWVGTIAADDDYGRPGIEKFREEAEERDIC
IDFSELISQYSDEEEIQHVVEVIQNSTAKVIVVFSSGPDLEPLIKEIVRRNITGKIWLA
SEAWASSSLIAMPQYFHVVGGTIGFALKAGQIPGFREFLKKVHPRKSVHNGFAKEFWEE
TFNCHLQEGAKGPLPVDTFLRGHEESGDRFSNSSTAFRPLCTGDENISSVETPYIDYTH
LRISYNVYLAVYSIAHALQDIYTCLPGRGLFTNGSCADIKKVEAWQVLKHLRHLNFTNN
MGEQVTFDECGDLVGNYSIINWHLSPEDGSIVFKEVGYYNVYAKKGERLFINEEKILWS
GFSREPLTFVLSVLQVPFSNCSRDCLAGTRKGIIEGEPTCCFECVECPDGEYSDETDAS
ACNKCPDDFWSNENHTSCIAKEIEFLSWTEPFGIALTLFAVLGIFLTAFVLGVFIKFRN
TPIVKATNRELSYLLLFSLLCCFSSSLFFIGEPQDWTCRLRQPAFGISFVLCISCILVK
TNRVLLVFEAKIPTSFHRKWWGLNLQFLLVFLCTFMQIVICVIWLYTAPPSSYRNQELE
DEIIFITCHEGSLMALGFLIGYTCLLAAICFFFAFKSRKLPENFNEAKFITFSMLIFFI
VWISFIPAYASTYGKFVSAVEVIAILAASFGLLACIFFNKIYIILFKPSRNTIEEVRCS
TAAHAFKVAARATLRRSNVSRKRSSSLGGSTGSTPSSSISSKSNSEDPFPQPERQKQQQ
PLALTQQEQQQQPLTLPQQQRSQQQPRCKQKVIFGSGTVTFSLSFDEPQKNAMAHRNST
HQNSLEAQKSSDTLTRHQPLLPLQCGETDLDLTVQETGLQGPVGGDQRPEVEDPEELSP
ALVVSSSQSFVISGGGSTVTENVVNS
mGluR targeting (mGluR1; bolded: mGluR1.
Underlined: R-CatchER)
SEQ ID NO: 39
MVGLLLFFFPAIFLEVSLLPRSPGRKVLLAGASSQRSVARMDGDVIIGALFSVHHQPPA
EKVPERKCGEIREQYGIQRVEAMFHTLDKINADPVLLPNITLGSEIRDSCWHSSVALEQ
SIEFIRDSLISIRDEKDGINRCLPDGQSLPPGRTKKPIAGVIGPGSSSVAIQVQNLLQL
FDIPQIAYSATSIDLSDKTLYKYFLRVVPSDTLQARAMLDIVKRYNWTYVSAVHTEGNY
GESGMDAFKELAAQEGLCIAHSDKIYSNAGEKSFDRLLRKLRERLPKARVVVCFCEGMT
VRGLLSAMRRLGVVGEFSLIGSDGWADRDEVIEGYEVEANGGITIKLQSPEVRSFDDYF
LKLRLDTNTRNPWFPEFWQHRFQCRLPGHLLENPNFKRICTGNESLEENYVQDSKMGFV
INAIYAMAHGLQNMHHALCPGHVGLCDAMKPIDGSKLLDFLIKSSFIGVSGEEVWFDEK
GDAPGRYDIMNLQYTEANRYDYVHVGTWHEGVLNIDDYKIQMNKSGVVRSVCSEPCLKG
QIKVIRKGEVSCCWICTACKENEYVQDEFTCKACDLGWWPNADLTGCEPIPVRYLEWSN
IESIIAIAFSCLGILVTLFVTLIFVLYRDTPVVKSSSRELCYIILAGIFLGYVCPFTLI
AKPTTTSCYLQRLLVGLSSAMCYSALVTKTNRIARILAGSKKKICTRKPRFMSAWAQVI
IASILISVQLTLVVTLIIMEPPMPILSYPSIKEVYLICNTSNLGVVAPLGYNGLLIMSC
TYYAFKTRNVPANFNEAKYIAFTMYTTCIIWLAFVPIYFGSNYKIITTCFAVSLSVTVA
LGCMFTPKMYIIIAKPERNVRSAFTTSDVVRMHVGDGKLPCRSNTFLNIFRRKKAGAGN
ANSNGKSVSWSEPGGGQVPKGQHMWHRLSVHVKTNETACNQTAVIKPLTKSYQGSGKSL
TFSDTSTKTLYNVEEEEDAQPIRFSPPGSPSMVVHRRVPSAATTPPLPSHLTAEETPLF
LAEPALPKGLPPPLQQQQQPPPQQKSLMDQLQGVVSNFSTAIPDFHAVLAGPGGPGNGL
RSLYPPPPPPQHLQMLPLQLSTFGEELVSPPADDDDDSERFKLLQEYVYEHEREGNTEE
DELEEEEEDLQAASKLTPDDSPALTPPSPFRDSVASGSSVPSSPVSESVLCTPPNVSYA
SVILRDYKQSSSTLMVSKGEENNMAIIKEFMRFKVHMEGSVNGHEFEIEGEGEGRPYEA
FQTAKLKVTKGGPLPFAWDILSPQFMYGSKVYIKHPADIPDYFKLSFPEGFRWERVMNF
EDGGIIHVNQDSSLQDGVFIYKVKLRGTNFPSDGPVMQKKTMGWEESEERMYPEDGALK
SEIKKRLKLKDGGHYAAEVKTTYKAKKPVQLPGAYIVDIDLDIVSHNEDYTIVEQYEDA
EGRHSTGGMDELYK
mGluR1 targeting sequence
SEQ ID NO: 40
MVGLLLFFFPAIFLEVSLLPRSPGRKVLLAGASSQRSVARMDGDVIIGALFSVHHQPPA
EKVPERKCGEIREQYGIQRVEAMFHTLDKINADPVLLPNITLGSEIRDSCWHSSVALEQ
SIEFIRDSLISIRDEKDGINRCLPDGQSLPPGRTKKPIAGVIGPGSSSVAIQVQNLLQL
FDIPQIAYSATSIDLSDKTLYKYFLRVVPSDTLQARAMLDIVKRYNWTYVSAVHTEGNY
GESGMDAFKELAAQEGLCIAHSDKIYSNAGEKSFDRLLRKLRERLPKARVVVCFCEGMT
VRGLLSAMRRLGVVGEFSLIGSDGWADRDEVIEGYEVEANGGITIKLQSPEVRSFDDYF
LKLRLDTNTRNPWFPEFWQHRFQCRLPGHLLENPNFKRICTGNESLEENYVQDSKMGFV
INAIYAMAHGLQNMHHALCPGHVGLCDAMKPIDGSKLLDFLIKSSFIGVSGEEVWFDEK
GDAPGRYDIMNLQYTEANRYDYVHVGTWHEGVLNIDDYKIQMNKSGVVRSVCSEPCLKG
QIKVIRKGEVSCCWICTACKENEYVQDEFTCKACDLGWWPNADLTGCEPIPVRYLEWSN
IESIIAIAFSCLGILVTLFVTLIFVLYRDTPVVKSSSRELCYIILAGIFLGYVCPFTLI
AKPTTTSCYLQRLLVGLSSAMCYSALVTKTNRIARILAGSKKKICTRKPRFMSAWAQVI
IASILISVQLTLVVTLIIMEPPMPILSYPSIKEVYLICNTSNLGVVAPLGYNGLLIMSC
TYYAFKTRNVPANFNEAKYIAFTMYTTCIIWLAFVPIYFGSNYKIITTCFAVSLSVTVA
LGCMFTPKMYIIIAKPERNVRSAFTTSDVVRMHVGDGKLPCRSNTFLNIFRRKKAGAGN
ANSNGKSVSWSEPGGGQVPKGQHMWHRLSVHVKTNETACNQTAVIKPLTKSYQGSGKSL
TFSDTSTKTLYNVEEEEDAQPIRFSPPGSPSMVVHRRVPSAATTPPLPSHLTAEETPLF
LAEPALPKGLPPPLQQQQQPPPQQKSLMDQLQGVVSNFSTAIPDFHAVLAGPGGPGNGL
RSLYPPPPPPQHLQMLPLQLSTFGEELVSPPADDDDDSERFKLLQEYVYEHEREGNTEE
DELEEEEEDLQAASKLTPDDSPALTPPSPFRDSVASGSSVPSSPVSESVLCTPPNVSYA
SVILRDYKQSSSTL,
TRP channel targeting (bolded: PKD2 targeting,
underlined: R-CatchER)
SEQ ID NO: 41
MVNSSRVQPQQPGDAKRPPAPRAPDPGRLMAGCAAVGASLAAPGGLCEQRGLEIEMQRI
RQAAARDPPAGAAASPSPPLSSCSRQAWSRDNPGFEAEEEEEEVEGEEGGMVVEMDVEW
RPGSRRSAASSAVSSVGARSRGLGGYHGAGHPSGRRRRREDQGPPCPSPVGGGDPLHRH
LPLEGQPPRVAWAERLVRGLRGLWGTRLMEESSTNREKYLKSVLRELVTYLLFLIVLCI
LTYGMMSSNVYYYTRMMSQLFLDTPVSKTEKTNFKTLSSMEDFWKFTEGSLLDGLYWKM
QPSNQTEADNRSFIFYENLLLGVPRIRQLRVRNGSCSIPQDLRDEIKECYDVYSVSSED
RAPFGPRNGTAWIYTSEKDLNGSSHWGIIATYSGAGYYLDLSRTREETAAQVASLKKNV
WLDRGTRATFIDFSVYNANINLFCVVRLLVEFPATGGVIPSWQFQPLKLIRYVTTFDFF
LAACEIIFCFFIFYYVVEEILEIRIHKLHYFRSFWNCLDVVIVVLSVVAIGINIYRTSN
VEVLLQFLEDQNTFPNFEHLAYWQIQFNNIAAVTVFFVWIKLFKFINFNRTMSQLSTTM
SRCAKDLFGFAIMFFIIFLAYAQLAYLVFGTQVDDFSTFQECIFTQFRIILGDINFAEI
EEANRVLGPIYFTTFVFFMFFILLNMFLAIINDTYSEVKSDLAQQKAEMELSDLIRKGY
HKALVKLKLKKNTVDDISESLRQGGGKLNFDELRQDLKGKGHTDAEIEAIFTKYDQDGD
QELTEHEHQQMRDDLEKEREDLDLDHSSLPRPMSSRSFPRSLDDSEEDDDEDSGHSSRR
RGSISSGVSYEEFQVLVRRVDRMEHSIGSIVSKIDAVIVKLEIMERAKLKRREVLGRLL
DGVAEDERLGRDSEIHREQMERLVREELERWESDDAASQISHGLGTPVGLNGQPRPRSS
RPSSSQSTEGMEGAGGNGSSNVHVMVSKGEENNMAIIKEFMRFKVHMEGSVNGHEFEIE
GEGEGRPYEAFQTAKLKVTKGGPLPFAWDILSPQFMYGSKVYIKHPADIPDYFKLSFPE
GFRWERVMNFEDGGIIHVNQDSSLQDGVFIYKVKLRGTNFPSDGPVMQKKTMGWEESEE
RMYPEDGALKSEIKKRLKLKDGGHYAAEVKTTYKAKKPVQLPGAYIVDIDLDIVSHNED
YTIVEQYEDAEGRHSTGGMDELYK,
TRP channel targeting (PKD2 targeting)
SEQ ID NO: 42
MVNSSRVQPQQPGDAKRPPAPRAPDPGRLMAGCAAVGASLAAPGGLCEQRGLEIEMQRI
RQAAARDPPAGAAASPSPPLSSCSRQAWSRDNPGFEAEEEEEEVEGEEGGMVVEMDVEW
RPGSRRSAASSAVSSVGARSRGLGGYHGAGHPSGRRRRREDQGPPCPSPVGGGDPLHRH
LPLEGQPPRVAWAERLVRGLRGLWGTRLMEESSTNREKYLKSVLRELVTYLLFLIVLCI
LTYGMMSSNVYYYTRMMSQLFLDTPVSKTEKTNFKTLSSMEDFWKFTEGSLLDGLYWKM
QPSNQTEADNRSFIFYENLLLGVPRIRQLRVRNGSCSIPQDLRDEIKECYDVYSVSSED
RAPFGPRNGTAWIYTSEKDLNGSSHWGIIATYSGAGYYLDLSRTREETAAQVASLKKNV
WLDRGTRATFIDFSVYNANINLFCVVRLLVEFPATGGVIPSWQFQPLKLIRYVTTFDFF
LAACEIIFCFFIFYYVVEEILEIRIHKLHYFRSFWNCLDVVIVVLSVVAIGINIYRTSN
VEVLLQFLEDQNTFPNFEHLAYWQIQFNNIAAVTVFFVWIKLFKFINFNRTMSQLSTTM
SRCAKDLFGFAIMFFIIFLAYAQLAYLVFGTQVDDFSTFQECIFTQFRIILGDINFAEI
EEANRVLGPIYFTTFVFFMFFILLNMFLAIINDTYSEVKSDLAQQKAEMELSDLIRKGY
HKALVKLKLKKNTVDDISESLRQGGGKLNFDELRQDLKGKGHTDAEIEAIFTKYDQDGD
QELTEHEHQQMRDDLEKEREDLDLDHSSLPRPMSSRSFPRSLDDSEEDDDEDSGHSSRR
RGSISSGVSYEEFQVLVRRVDRMEHSIGSIVSKIDAVIVKLEIMERAKLKRREVLGRLL
DGVAEDERLGRDSEIHREQMERLVREELERWESDDAASQISHGLGTPVGLNGQPRPRSS
RPSSSQSTEGMEGAGGNGSSNVHV,
(NMDAR targeting (e.g. GluN2A; bolded: GluN2A;
underlined: R-CatchER)
SEQ ID NO: 43
MGRLGYWTLLVLPALLVWRDPAQNAAAEKGPPALNIAVLLGHSHDVTERELRNLWGPEQ
ATGLPLDVNVVALLMNRTDPKSLITHVCDLMSGARIHGLVFGDDTDQEAVAQMLDFISS
QTFIPILGIHGGASMIMADKDPTSTFFQFGASIQQQATVMLKIMQDYDWHVFSLVTTIF
PGYRDFISFIKTTVDNSFVGWDMQNVITLDTSFEDAKTQVQLKKIHSSVILLYCSKDEA
VLILSEARSLGLTGYDFFWIVPSLVSGNTELIPKEFPSGLISVSYDDWDYSLEARVRDG
LGILTTAASSMLEKFSYIPEAKASCYGQAEKPETPLHTLHQFMVNVTWDGKDLSFTEEG
YQVHPRLVVIVLNKDREWEKVGKWENQTLSLRHAVWPRYKSFSDCEPDDNHLSIVTLEE
APFVIVEDIDPLTETCVRNTVPCRKFVKINNSTNEGMNVKKCCKGFCIDILKKLSRTVK
FTYDLYLVTNGKHGKKVNNVWNGMIGEVVYQRAVMAVGSLTINEERSEVVDFSVPFVET
GISVMVSRSNGTVSPSAFLEPFSASVWVMMFVMLLIVSAIAVFVFEYFSPVGYNRNLAK
GKAPHGPSFTIGKAIWLLWGLVENNSVPVQNPKGTTSKIMVSVWAFFAVIFLASYTANL
AAFMIQEEFVDQVTGLSDKKFQRPHDYSPPFRFGTVPNGSTERNIRNNYPYMHQYMTRF
NQRGVEDALVSLKTGKLDAFIYDAAVLNYKAGRDEGCKLVTIGSGYIFASTGYGIALQK
GSPWKRQIDLALLQFVGDGEMEELETLWLTGICHNEKNEVMSSQLDIDNMAGVFYMLAA
AMALSLITFIWEHLFYWKLRFCFTGVCSDRPGLLFSISRGIYSCIHGVHIEEKKKSPDF
NLTGSQSNMLKLLRSAKNISNMSNMNSSRMDSPKRATDFIQRGSLIVDMVSDKGNLIYS
DNRSFQGKDSIFGDNMNELQTFVANRHKDNLSNYVFQGQHPLTLNESNPNTVEVAVSTE
SKGNSRPRQLWKKSMESLRQDSLNQNPVSQRDEKTAENRTHSLKSPRYLPEEVAHSDIS
ETSSRATCHREPDNNKNHKTKDNFKRSMASKYPKDCSDVDRTYMKTKASSPRDKIYTID
GEKEPSFHLDPPQFVENITLPENVGFPDTYQDHNENFRKGDSTLPMNRNPLHNEDGLPN
NDQYKLYAKHFTLKDKGSPHSEGSDRYRQNSTHCRSCLSNLPTYSGHFTMRSPFKCDAC
LRMGNLYDIDEDQMLQETGNPATREEVYQQDWSQNNALQFQKNKLRINRQHSYDNILDK
PREIDLSRPSRSISLKDRERLLEGNLYGSLFSVPSSKLLGNKSSLFPQGLEDSKRSKSL
LPDHASDNPFLHTYGDDQRLVIGRCPSDPYKHSLPSQAVNDSYLRSSLRSTASYCSRDS
RGHSDVYISEHVMPYAANKNTMYSTPRVLNSCSNRRVYKKMPSIESDVMVSKGEENNMA
IIKEFMRFKVHMEGSVNGHEFEIEGEGEGRPYEAFQTAKLKVTKGGPLPFAWDILSPQF
MYGSKVYIKHPADIPDYFKLSFPEGFRWERVMNFEDGGIIHVNQDSSLQDGVFIYKVKL
RGTNFPSDGPVMQKKTMGWEESEERMYPEDGALKSEIKKRLKLKDGGHYAAEVKTTYKA
KKPVQLPGAYIVDIDLDIVSHNEDYTIVEQYEDAEGRHSTGGMDELYK
(NMDAR targeting (e.g. GluN2A)
SEQ ID NO: 44
MGRLGYWTLLVLPALLVWRDPAQNAAAEKGPPALNIAVLLGHSHDVTERELRNLWGPEQ
ATGLPLDVNVVALLMNRTDPKSLITHVCDLMSGARIHGLVFGDDTDQEAVAQMLDFISS
QTFIPILGIHGGASMIMADKDPTSTFFQFGASIQQQATVMLKIMQDYDWHVFSLVTTIF
PGYRDFISFIKTTVDNSFVGWDMQNVITLDTSFEDAKTQVQLKKIHSSVILLYCSKDEA
VLILSEARSLGLTGYDFFWIVPSLVSGNTELIPKEFPSGLISVSYDDWDYSLEARVRDG
LGILTTAASSMLEKFSYIPEAKASCYGQAEKPETPLHTLHQFMVNVTWDGKDLSFTEEG
YQVHPRLVVIVLNKDREWEKVGKWENQTLSLRHAVWPRYKSFSDCEPDDNHLSIVTLEE
APFVIVEDIDPLTETCVRNTVPCRKFVKINNSTNEGMNVKKCCKGFCIDILKKLSRTVK
FTYDLYLVTNGKHGKKVNNVWNGMIGEVVYQRAVMAVGSLTINEERSEVVDFSVPFVET
GISVMVSRSNGTVSPSAFLEPFSASVWVMMFVMLLIVSAIAVFVFEYFSPVGYNRNLAK
GKAPHGPSFTIGKAIWLLWGLVFNNSVPVQNPKGTTSKIMVSVWAFFAVIFLASYTANL
AAFMIQEEFVDQVTGLSDKKFQRPHDYSPPFRFGTVPNGSTERNIRNNYPYMHQYMTRF
NQRGVEDALVSLKTGKLDAFIYDAAVLNYKAGRDEGCKLVTIGSGYIFASTGYGIALQK
GSPWKRQIDLALLQFVGDGEMEELETLWLTGICHNEKNEVMSSQLDIDNMAGVFYMLAA
AMALSLITFIWEHLFYWKLRFCFTGVCSDRPGLLFSISRGIYSCIHGVHIEEKKKSPDF
NLTGSQSNMLKLLRSAKNISNMSNMNSSRMDSPKRATDFIQRGSLIVDMVSDKGNLIYS
DNRSFQGKDSIFGDNMNELQTFVANRHKDNLSNYVFQGQHPLTLNESNPNTVEVAVSTE
SKGNSRPRQLWKKSMESLRQDSLNQNPVSQRDEKTAENRTHSLKSPRYLPEEVAHSDIS
ETSSRATCHREPDNNKNHKTKDNFKRSMASKYPKDCSDVDRTYMKTKASSPRDKIYTID
GEKEPSFHLDPPQFVENITLPENVGFPDTYQDHNENFRKGDSTLPMNRNPLHNEDGLPN
NDQYKLYAKHFTLKDKGSPHSEGSDRYRQNSTHCRSCLSNLPTYSGHFTMRSPFKCDAC
LRMGNLYDIDEDQMLQETGNPATREEVYQQDWSQNNALQFQKNKLRINRQHSYDNILDK
PREIDLSRPSRSISLKDRERLLEGNLYGSLFSVPSSKLLGNKSSLFPQGLEDSKRSKSL
LPDHASDNPFLHTYGDDQRLVIGRCPSDPYKHSLPSQAVNDSYLRSSLRSTASYCSRDS
RGHSDVYISEHVMPYAANKNTMYSTPRVLNSCSNRRVYKKMPSIESDV
AMPAR targeting (e.g. GluR1 bolded: GluR1;
underlined: R-CatchER)
SEQ ID NO: 45
QHIFAFFCTGFLGAVVGANFPNNIQIGGLFPNQQSQEHAAFRFALSQLTEPPKLLPQID
IVNISDSFEMTYRFCSQFSKGVYAIFGFYERRTVNMLTSFCGALHVCFITPSFPVDTSN
QFVLQLRPELQDALISIIDHYKWQKFVYIYDADRGLSVLQKVLDTAAEKNWQVTAVNIL
TTTEEGYRMLFQDLEKKKERLVVVDCESERLNAILGQIIKLEKNGIGYHYILANLGFMD
IDLNKFKESGANVTGFQLVNYTDTIPAKIMQQWKNSDARDHTRVDWKRPKYTSALTYDG
VKVMAEAFQSLRRQRIDISRRGNAGDCLANPAVPWGQGIDIQRALQQVRFEGLTGNVQF
NEKGRRTNYTLHVIEMKHDGIRKIGYWNEDDKFVPAATDAQAGGDNSSVQNRTYIVTTI
LEDPYVMLKKNANQFEGNDRYEGYCVELAAEIAKHVGYSYRLEIVSDGKYGARDPDTKA
WNGMVGELVYGRADVAVAPLTITLVREEVIDFSKPFMSLGISIMIKKPQKSKPGVFSFL
DPLAYEIWMCIVFAYIGVSVVLFLVSRFSPYEWHSEEFEEGRDQTTSDQSNEFGIFNSL
WFSLGAFMQQGCDISPRSLSGRIVGGVWWFFTLIIISSYTANLAAFLTVERMVSPIESA
EDLAKQTEIAYGTLEAGSTKEFFRRSKIAVFEKMWTYMKSAEPSVFVRTTEEGMIRVRK
SKGKYAYLLESTMNEYIEQRKPCDTMKVGGNLDSKGYGIATPKGSALRNPVNLAVLKLN
EQGLLDKLKNKWWYDKGECGSGGGDSKDKTSALSLSNVAGVFYILIGGLGLAMLVALIE
FCYKSRSESKRMKGFCLIPQQSINEAIRTSTLPRNSGAGASSGGSGENGRVVSHDFPKS
MQSIPCMSHSSGMPLGATGLMVSKGEENNMAIIKEFMRFKVHMEGSVNGHEFEIEGEGE
GRPYEAFQTAKLKVTKGGPLPFAWDILSPOFMYGSKVYIKHPADIPDYFKLSFPEGFRW
ERVMNFEDGGIIHVNQDSSLQDGVFIYKVKLRGTNFPSDGPVMQKKTMGWEESEERMYP
EDGALKSEIKKRLKLKDGGHYAAEVKTTYKAKKPVOLPGAYIVDIDLDIVSHNEDYTIV
EQYEDAEGRHSTGGMDELYK
AMPAR targeting (e.g. GluR1)
SEQ ID NO: 46
QHIFAFFCTGFLGAVVGANFPNNIQIGGLFPNQQSQEHAAFRFALSQLTEPPKLLPQID
IVNISDSFEMTYRFCSQFSKGVYAIFGFYERRTVNMLTSFCGALHVCFITPSFPVDTSN
QFVLQLRPELQDALISIIDHYKWQKFVYIYDADRGLSVLQKVLDTAAEKNWQVTAVNIL
TTTEEGYRMLFQDLEKKKERLVVVDCESERLNAILGQIIKLEKNGIGYHYILANLGFMD
IDLNKFKESGANVTGFQLVNYTDTIPAKIMQQWKNSDARDHTRVDWKRPKYTSALTYDG
VKVMAEAFQSLRRQRIDISRRGNAGDCLANPAVPWGQGIDIQRALQQVRFEGLTGNVQF
NEKGRRTNYTLHVIEMKHDGIRKIGYWNEDDKFVPAATDAQAGGDNSSVQNRTYIVTTI
LEDPYVMLKKNANQFEGNDRYEGYCVELAAEIAKHVGYSYRLEIVSDGKYGARDPDTKA
WNGMVGELVYGRADVAVAPLTITLVREEVIDFSKPFMSLGISIMIKKPQKSKPGVFSFL
DPLAYEIWMCIVFAYIGVSVVLFLVSRFSPYEWHSEEFEEGRDQTTSDQSNEFGIFNSL
WFSLGAFMQQGCDISPRSLSGRIVGGVWWFFTLIIISSYTANLAAFLTVERMVSPIESA
EDLAKQTEIAYGTLEAGSTKEFFRRSKIAVFEKMWTYMKSAEPSVFVRTTEEGMIRVRK
SKGKYAYLLESTMNEYIEQRKPCDTMKVGGNLDSKGYGIATPKGSALRNPVNLAVLKLN
EQGLLDKLKNKWWYDKGECGSGGGDSKDKTSALSLSNVAGVFYILIGGLGLAMLVALIE
FCYKSRSESKRMKGFCLIPQQSINEAIRTSTLPRNSGAGASSGGSGENGRVVSHDFPKS
MQSIPCMSHSSGMPLGATGL
jGCaMP7 (e.g., jGCaMP7s)
SEQ ID NO: 47
MGSHHHHHHGMASMTGGQQMGRDLYDDDDKDLATMVDSSRRKWNKTGHAVRVIGRLSSL
ENVYIKADKQKNGIKANFHIRHNIEDGGVQLAYHYQQNTPIGDGPVLLPDNHYLSVQSK
LSKDPNEKRDHMVLLEFVTAAGITLGMDELYKGGTGGSMVSKGEELFTGVVPILVELDG
DVNGHKFSVSGEGEGDATYGKLTLKFICTTGKLPVPWPTLVTTLTYGVQCFSRYPDHMK
QHDFFKSAMPEGYIQERTIFFKDDGNYKTRAEVKFEGDTLVNRIELKGIDFKEDGNILG
HKLEYNLPDQLTEEQIAEFKELFSLFDKDGDGTITTKELGTVMRSLGQNPTEAELQDMI
NEVDADGDGTIDFPEFLTMMARKMKYRDTEEEIREAFGVFDKDGNGYISAAELRHVMTN
LGEKLTDEEVDEMIREADIDGDGQVNYEEFVQMMTA
Endosome/lysosome targeting (e.g., TRPML1)
SEQ ID NO: 48
MATPAGRRASETERLLTPNPGYGTQVGTSPAPTTPTEEEDLRRRLKYFFMSPCDKFRAK
GRKPCKLMLQVVKILVVTVQLILFGLSNQLVVTFREENTIAFRHLFLLGYSDGSDDTFA
AYTQEQLYQAIFYAVDQYLILPEISLGRYAYVRGGGGPWANGSALALCQRYYHRGHVDP
ANDTFDIDPRVVTDCIQVDPPDRPPDIPSEDLDFLDGSASYKNLTLKFHKLINVTIHFQ
LKTINLQSLINNEIPDCYTFSILITFDNKAHSGRIPIRLETKTHIQECKHPSVSRHGDN
SFRLLFDVVVILTCSLSFLLCARSLLRGFLLQNEFVVFMWRRRGREISLWERLEFVNGW
YILLVTSDVLTISGTVMKIGIEAKNLASYDVCSILLGTSTLLVWVGVIRYLTFFHKYNI
LIATLRVALPSVMRFCCCVAVIYLGYCFCGWIVLGPYHVKFRSLSMVSECLFSLINGDD
MFVTFAAMQAQQGHSSLVWLFSQLYLYSFISLFIYMVLSLFIALITGAYDTIKHPGGTG
TEKSELQAYIEQCQDSPTSGKFRRGSGSACSLFCCCGRDSPEDHSLLVN
G-CatchER (N149E/E204D/E223D)
SEQ ID NO: 49
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHEVYITADKQKNGIKANFKIRHNIEDGSV
QLADHYQQNTPIGDGPVLLPDNHYLDTDSALSKDPNEKRDHMVLLEDVEAAGITLGMDE
LYK
G-CatchER (N149E/E204D)
SEQ ID NO: 50
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHEVYITADKQKNGIKANFKIRHNIEDGSV
QLADHYQQNTPIGDGPVLLPDNHYLDTDSALSKDPNEKRDHMVLLEEVEAAGITLGMDE
LYK
G-CatchER (E223D)
SEQ ID NO: 51
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHNVYITADKQKNGIKANFKIRHNIEDGSV
QLADHYQQNTPIGDGPVLLPDNHYLDTESALSKDPNEKRDHMVLLEDVEAAGITLGMDE
LYK
G-CatchER (E204D)
SEQ ID NO: 52
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHNVYITADKQKNGIKANFKIRHNIEDGSV
QLADHYQQNTPIGDGPVLLPDNHYLDTDSALSKDPNEKRDHMVLLEEVEAAGITLGMDE
LYK
G-CatchER (E147D)
SEQ ID NO: 53
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNDHNVYITADKQKNGIKANFKIRHNIEDGSV
QLADHYQQNTPIGDGPVLLPDNHYLDTESALSKDPNEKRDHMVLLEEVEAAGITLGMDE
LYK
G-CatchER (N149D)
SEQ ID NO: 54
MVSKGEELFTGVVPILVELDGDVNGHKFSVSGEGEGDATYGKLTLKFICTTGKLPVPWP
TLVTTLTYGVQCFSRYPDHMKQHDFFKSAMPEGYVQERTIFFKDDGNYKTRAEVKFEGD
TLVNRIELKGIDFKEDGNILGHKLEYNYNEHDVYITADKQKNGIKANFKIRHNIEDGSV
QLADHYQQNTPIGDGPVLLPDNHYLDTESALSKDPNEKRDHMVLLEEVEAAGITLGMDE
LYK

1.-28. (canceled)

29. A polypeptide metal ion sensor comprising an engineered red-fluorescent polypeptide having a heterologous metal ion binding site, wherein said engineered red-fluorescent polypeptide is a variant amino acid sequence of SEQ ID NO: 11 having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species.

30. The polypeptide metal ion sensor of claim 29, wherein the amino acid sequence of said engineered red-fluorescent polypeptide comprises a sequence having at least 95% identity to SEQ ID NO: 12.

31. The polypeptide metal ion sensor of claim 29, wherein the amino acid sequence of said engineered red-fluorescent polypeptide consists of SEQ ID NO: 12.

32. The polypeptide metal ion sensor of claim 29, wherein said polypeptide metal ion sensor comprises at least one targeting moiety that specifically recognizes a structural feature of a cell or tissue, or a target biomolecule.

33. The polypeptide metal ion sensor of claim 32, wherein said at least one targeting moiety specifically recognizes a target component of an endoplasmic reticulum or a sarcoplasmic reticulum of a cell.

34. The polypeptide metal ion sensor of claim 32, wherein said at least one targeting moiety specifically recognizes a target component of the mitochondria of a cell.

35. The polypeptide metal ion sensor of claim 32, wherein the targeting moiety comprises a sequence at least about 95% identical to SEQ ID NO: 33 and/or 34.

36. The polypeptide metal ion sensor of claim 32, wherein said at least one targeting moiety specifically recognizes a transient receptor potential (TRP) channel, an N-methyl-D-aspartate (NMDA) receptor, and an α-amino-3-hydroxy-5-methyl-4-isoxazolepropionic acid (AMPA) receptor.

37. The polypeptide metal ion sensor of claim 32, wherein the targeting moiety comprises a sequence at least 95% identical to SEQ ID NO: 38, 40, 42, 44, or 46.

38. The polypeptide metal ion sensor of claim 32, wherein said at least one targeting moiety specifically recognizes a target polypeptide.

39. The polypeptide metal ion sensor of claim 29, wherein said metal ion binding site specifically binds to a metal ion, wherein the metal is a lanthanide metal, an alkaline earth metal, lead, cadmium, or a transition metal.

40. The polypeptide metal ion sensor of claim 39, wherein the lanthanide metal is selected from the group consisting of lanthanum, gadolinium, and terbium.

41. The polypeptide metal ion sensor of claim 39, wherein the alkaline earth metal is selected from the group consisting of calcium, strontium, and magnesium.

42. The polypeptide metal ion sensor of claim 39, wherein the transition metal is selected from the group consisting of zinc and manganese.

43. A method of detecting metal ions in a biological sample, comprising: (i) providing a polypeptide metal ion sensor comprising an engineered red-fluorescent polypeptide having a heterologous metal ion binding site, wherein said engineered red-fluorescent polypeptide is a variant of amino acid sequence SEQ ID NO: 11 and having the amino acid substitutions corresponding to A145E, K198D, and/or R216D and, when having a metal ion species bound thereto, exhibits an elevated fluorescence output compared to the polypeptide SEQ ID NO: 11 binding to the same metal ion species; (ii) delivering the polypeptide metal ion sensor or an expression vector having a nucleic acid sequence encoding said metal sensor to a biological sample; (iii) detecting a first spectroscopic signal emitted by said sensor; (iv) generating a physiological or cellular change in the biological sample; and (v) detecting a second spectroscopic signal emitted by said sensor after step (iii).

44. The method of claim 43, wherein a detectable change in at least one of a wavelength, an intensity, and/or lifetime between the first and second spectroscopic signals indicates a change in the rate of release, refill, or cellular concentration of a metal ion in the sample.

45. (canceled)

46. (canceled)

47. The method of claim 43, wherein the detectable change in the signal intensity provides a quantitative measurement of the metal ion in the sample.

48. The method of claim 43, wherein the biological sample is a cell or tissue of an animal or human subject, or a cell or tissue isolated from an animal or human subject.

49. The method of claim 43, wherein the spectroscopic signal generated when a metal ion is bound to said sensor is used to generate an image.

50.-56. (canceled)

57. The method of claim 43, further comprising a step of delivering to the biological sample a second polypeptide metal ion sensor comprising an engineered green-fluorescent polypeptide having a heterologous metal ion binding site, wherein said metal ion binding site specifically binds to a metal ion in the cytosol of the biological sample.

58. The method of claim 57, wherein the second polypeptide metal ion sensor is a calmodulin-based sensor.

59. The method of claim 58, wherein the second polypeptide metal ion sensor is jGCaMP7, or a sequence at least 90% identical to jGCaMP7.

60. A polypeptide metal ion sensor comprising an engineered red-fluorescent polypeptide having a heterologous metal ion binding site, wherein said engineered red-fluorescent polypeptide is a variant amino acid sequence of SEQ ID NO: 11 having the amino acid substitutions corresponding to A145E, K198D, and/or R216E and an amino acid substitution at residue K163.

61. The polypeptide metal ion sensor of claim 60, wherein the amino acid substitute at residue K163 is K163Q, K163M, or K163L.

62. The polypeptide metal ion sensor of claim 61, wherein the amino acid substitute at residue K163 is K163L.

63. The polypeptide metal ion sensor of claim 60, further comprising a mitochondria targeting sequence.

64. The polypeptide metal ion sensor of claim 63, wherein the mitochondria targeting sequence comprises a sequence having at least 95% identity to SEQ ID NO: 33 and/or 34.
